(19) United States
(12) Patent Application Publication
(10) Pub. No.: US 2011/0236367 A1
(43) Pub. Date: Sep. 29, 2011

(54) RAT CATHESPIN DIPEPTIDYL PEPTIDASE I (DPPI): CRYSTAL STRUCTURE AND ITS USES

(75) Inventors: Johan Gotthardt OLSEN, Copenhagen (DK); Anders Kadziola, Hellerup (DK); Søren Weis Dahl, Rungsted Kyst (DK); Connie Lauritzen, Rødovre (DK); Sine Larsen, Hørsholm (DK); John Pedersen, Niva (DK); Dusan Turk, Ljubljana (SI); Marjetka Podobnik, Ljubljana-Polje (SI); Igor Stern, Ljubljana (SI)

(73) Assignee: TeleNav, Inc., Sunnyvale, CA (US)

(21) Appl. No.: 12/784,139

(22) Filed: May 20, 2010

Related U.S. Application Data
(63) Continuation-in-part of application No. 10/363,712, filed on Aug. 15, 2003, now Pat. No. 7,736,875.
(60) Provisional application No. 60/247,584, filed on Nov. 9, 2000.

(30) Foreign Application Priority Data
Sep. 8, 2000 (DK) PA 2000 01343

(51) Int. Cl. A61K 38/48; C12N 9/48; C12N 9/96; C07D 303/02; G01N 33/68; A61K 38/05; A61P 35/00; A61P 11/06; A61P 17/06; A61P 29/00; A61P 19/02; A61P 31/00; A61P 1/00; G06G 7/60; G06F 19/16

(52) U.S. Cl. 424/94.63; 435/212; 435/188; 549/512; 435/7.1; 514/21.91; 514/1.7; 514/18.6; 514/18.9; 514/19.3; 514/16.6; 514/1.5; 514/2.3; 703/11; 702/19

(57) ABSTRACT

The present invention relates to structural studies of dipeptidyl peptidase I (DPPI) proteins, modified dipeptidyl peptidase I (DPPI) proteins and DPPI co-complexes. Included in the present invention is a crystal of a dipeptidyl peptidase I (DPPI) and corresponding structural information obtained by X-ray crystallography from rat and human DPPI. In addition, this invention relates to methods for using structure co-ordinates of DDPI, mutants hereof and co-complexes, to design compounds that bind to the active site or accessory binding sites of DPPI and to design improved inhibitors of DPPI or homologues of the enzyme.

### INCORPORATION BY REFERENCE

This application is a continuation-in-part application of U.S. application Ser. No. 10/363,712, filed Aug. 15, 2003, now allowed, which is a §371 of PCT/DK01/00580, filed Sep. 6, 2001 and claims priority to Denmark Application No. PA 2000 01343 filed Sep. 8, 2000 and claims the benefit of U.S. Application No. 60/247,584, filed Nov. 9, 2000.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF INVENTION

The present invention relates generally to structural studies of dipeptidyl peptidase I (DPPI) proteins, modified dipeptidyl peptidase I (DPPI) proteins and DPPI co-complexes. Included in the present invention is a crystal of the dipeptidyl peptidase I (DPPI) and corresponding structural information obtained by X-ray crystallography. In addition, this invention relates to methods for using the structure co-ordinates of DPPI, mutants hereof and co-complexes to design compounds that bind to the active site or accessory binding sites of DPPI and to design improved inhibitors of DPPI or homologues of the enzyme.

### BACKGROUND OF INVENTION

Dipeptidyl peptidase I (DPPI, EC 3.4.14.1), previously known as dipeptidyl aminopeptidase I (DAPI), dipeptidyl transferase, cathepsin C and cathepsin J is a lysosomal cysteine exo-peptidase belonging to the papain family. DPPI is widely distributed in mammalian and bird tissues and the main sources of purification of the enzyme are liver and spleen. The cDNAs encoding rat, human, murine, bovine, dog and two Schistosome DPPIs have been cloned and sequenced and show that the enzyme is highly conserved. The human and rat DPPI cDNAs encode precursors (prepropPPI) comprising signal peptides of 24 residues, proregions of 205 (rat DPPI) or 206 (human DPPI) residues and catalytic domains of 233 residues which contain the catalytic residues and are 30-40% identical to the mature amino acid sequences of papain and a number of other cathepsins including cathepsins L, S, K, B and H.

The translated prepropPPI is processed into the mature form by at least four cleavages of the polypeptide chain. The signal peptide is removed during translocation or secretion of the proenzyme (propPPI) and a large N-terminal proregion fragment, which is retained in the mature enzyme, is separated from the catalytic domain by excision of a minor C-terminal part of the proregion, called the activation peptide. A heavy chain of about 164 residues and a light chain of about 69 residues are generated by cleavage of the catalytic domain.

Unlike the other members of the papain family, mature DPPI consists of four subunits, each composed of the N-terminal proregion fragment, the heavy chain and the light chain. Both the proregion fragment and the heavy chain are glycosylated.

DPPI catalyses excision of dipeptides from the N-terminus of protein and peptide substrates, except if (i) the amino group of the N-terminus is blocked, (ii) the site of cleavage is on either side of a proline residue, (iii) the N-terminal residue is lysine or arginine, or (iv) the structure of the peptide or protein prevents further digestion from the N-terminus.

DPPI is expressed in many tissues and has generally been associated with protein degradation in the lysosomes. More recently, DPPI has also been assigned an important role in the activation of many granule-associated serine proteinases, including cathepsin G and elastase from neutrophils, granzyme A, B and K from cytotoxic lymphocytes (CTL, NK and LAK cells) and chymase and tryptase from mast cells. These immune/inflammatory cell proteinases are translated as inactive zymogens and the final step in the conversion to their active forms is a DPPI-catalysed removal of an activation dipeptide from the N-terminus of the zymogens. DPPI−/− knock-out mice have been shown to exclusively accumulate the inactive, dipeptide extended proforms of the pro-apoptopic proteases granzyme A and B.

Many of the granule-associated proteases, which are activated by DPPI, serve important biological functions and inhibition of DPPI may thus be a general means of controlling the activities of these proteases.

Neutrophils cause considerable damage in a number of pathological conditions. When activated, neutrophils secrete destructive granular enzymes, including elastase and cathepsin G, and undergo oxidative bursts to release reactive oxygen intermediates. Numerous studies have been conducted on each of these activating agents in isolation. Pulmonary emphysema, cystic fibrosis and rheumatoid arthritis are just some examples of pathological conditions associated with the potent enzymes elastase and cathepsin G. Specifically, the imbalance in plasma levels of these two enzymes and their naturally occurring inhibitors, alpha 1-protease inhibitor and antichymotrypsin, may lead to severe and permanent tissue damage. These facts together with the shown relation between the induction of neutrophil activation and the activation and release of elastase and cathepsin G point to DPPI as an alternative target enzyme for therapeutic intervention against rheumatoid arthritis and related autoimmune diseases.

Cytotoxic lymphocytes play an important role in host-cell responses against viral and intracellular bacterial pathogens. They are also involved in anti-tumour responses, allograft rejection, and in a number of various autoimmune diseases. Though CTL, NK, and LAK cells kill via multiple mechanisms, evidence over the past few years have shown that two major pathways are responsible for the induction of target cell apoptosis. These are the Fax-FasL pathway and the granule exocytosis pathway.

Activated cytotoxic lymphocytes contain lytic granules, which are the hallmark of specialised killer cells. Among the proteins found in lytic granules are perforin and the highly related serine proteases of the granzyme family, including granzyme A, B and K. The importance of perforin and granzymes for cell-mediated cytotoxicity and apoptosis has been firmly established in several loss-of-function models.

Granzyme A and B knockout mice have shown that granzyme B is critical for the rapid induction of apoptosis in susceptible target cells, while granzyme A plays an important role in the late pathway of cytotoxicity. The above mentioned fact that DPPI−/− knock-out mice have been shown to exclusively accumulate the inactive proforms of granzyme A and B points to DPPI as an alternative target enzyme for therapeutic intervention and also provides a rationale for developing inhibitors against DPPI that could modulate immune responses against tumours, grafts, and various autoimmune diseases.

Mast cells are found in many tissues, but are present in greater numbers along the epithelial linings of the body, such as the skin, respiratory tract and gastrointestinal tract. Mast cells are also located in the perivascular tissue surrounding small blood vessels. This cell type can release a range of potent inflammatory mediators including cytokines, leukotrienes, prostaglandins, histamine and proteoglycans. Among the most abundant products of mast cell activation, though, are the serine proteases of the chymotrypsin family, tryptase and chymase. The use of in vivo models has provided confirmatory evidence that tryptases and chymases are important mediators of a number of mast cell mediated allergic, immunological and inflammatory diseases, including asthma, psoriasis, inflammatory bowel disease and atherosclerosis. For years, pharmaceutical companies have targeted the inhibition of tryptase and chymase as a drug intervention strategy.

However, the active sites and catalytic activities of tryptases and chymases closely resemble a number of other proteases of the same family and it has proven very difficult to design inhibitors that are at the same time sufficiently selective, potent, non-toxic and bioavailable. Furthermore, the large quantities of tryptases and chymases that are synthesised and released by mast cells make it difficult to ensure a continuous and satisfactory supply of inhibitors at the sites of release. The strong evidence associating tryptases and chymases with a number of mast cell mediated allergic, immunological and inflammatory diseases, and the fact that DPPI is needed for the activation of tryptase and chymase, outline DPPI as an alternative target enzyme for therapeutic intervention against the above mentioned mast cell diseases.

Low molecular weight substrates that mimic peptidyl inhibitors of DPPI, such as Gly-Phe- and Gly-Arg-diazomethyl ketones, chloromethyl ketones and fluoromethyl ketones have previously been reported. However, due to their peptidic nature and reactive groups, such inhibitors are typically characterised by undesirable pharmacological properties, such as poor oral absorption, poor stability, rapid metabolism and high toxicity.

Knowledge of the crystal structure co-ordinates and atomic details of DPPI, or its mutants or homologues or co-complexes, would facilitate or enable the design, computational evaluation, synthesis and use of DPPI inhibitors with improved properties as compared to the known peptidic DPPI inhibitors.

In addition to the interest in the unique structural and functional properties of DPPI, attention has also been turned to the technological applications of the enzyme.

By virtue of its restricted specificity, DPPI has been shown to be suitable for excision of certain extension peptides from the N-termini of recombinant proteins having a DPPI stop-point integrated in or placed in front of their N-terminal sequences. These properties of DPPI have been utilised to develop a specific and efficient method using recombinant DPPI variants for complete removal of a group of purification tags from the N-termini of target proteins. The addition of purification tags to the target protein is a simple and well-established approach for generating a novel affinity, making one-step purifications of recombinant proteins possible by using affinity chromatography. The combined processes of using purification tags for purification of recombinant proteins and DPPI for cleavage of the purification tag generating the desired N-terminal in the target protein (the DPPI/tag strategy), hold promises for use in large-scale productions of pharmaceutical proteins and peptide products. Its strength obviously is the simple overall design, the use of robust and inexpensive matrices, and the use of efficient enzymes.

In order to fully exploit the potential of this DPPI/tag strategy, it is thus desirable to alter the chemical, physical and enzymatic properties of DPPI to be able to use the enzyme in different condition, thereby making the DPPI/tag strategy more efficient, flexible and/or even more economically feasible.

Furthermore, besides its aminopeptidase activity, DPPI also displays a transferase activity, i.e. DPPI catalyses the transfer of dipeptide moieties from amides and esters of dipeptides to the N-terminal of unprotected peptides and proteins. This transferase activity of DPPI consequently bears a potential usage in methods for enzymatic synthesis and/or semisynthesis of peptides and proteins, but because of problems with the reverse (aminopeptidase) activity and substrate restrictions, transpeptidation by DPPI has been rarely used or exploited for peptide and protein synthesis.

The crystal structure of a number of cysteine peptidases of the papain family, including papain, chymopapain, actinidin, cathepsin B, and cathepsin have been known for many years, but despite DPPI being highly homologous to the other members of the papain family, and despite DPPI being available as purified and characterised preparation since 1960 (Metrione, R. M. et al, Biochemistry 5, 1597-1604, 1966; McDonnald J. K. et al, J. Biol. Chem. 244, 2693-2709, 1969), it has until now been impossible to obtain crystals of DPPI for solving the crystal structure of the enzyme.

Alternative interests have thus been focussed on trying to solve some of the structural features of DPPI through homology modelling, based on the known crystal structures of other cysteine peptidases of the papain family. However, although there are many resemblances to these other cysteine peptidases, it has not been possible to model the structure of DPPI because of very distinct differences. These differences include the oligomeric structure of DPPI, the detainment of the residual propart in the active enzyme and a unique chain cleavage pattern in active DPPI, features not present in and/or seen in the known crystal structures of the other cysteine peptidases of the papain family.

### OBJECT OF INVENTION

The object of the invention is a crystal structure of a dipeptidyl peptidase I (DPPI) protein, a modified dipeptidyl peptidase I (DPPI) protein, a protein comprising at least 37% identity with the amino acid sequence of rat DPPI, as shown in FIG. 1 and/or in SEQ ID NR. 1, or a DPPI co-complexe, and the use of the atomic co-ordinates of a said crystal structure obtained by X-ray crystallography, such as for designing inhibitors of DPPI and homologues of said enzyme.

Citation or identification of any document in this application is not an admission that such document is available as prior art to the present invention.

### SUMMARY OF INVENTION

Despite numerous unsuccessful attempts to determine the crystal structure, atomic co-ordinates and structural model of DPPI, the present invention surprisingly provides crystals of DPPI, which effectively diffract X-rays and thereby allow the determination of the atomic co-ordinates of the protein. The present invention furthermore provides the means to use this structural information as the basis for a design of new and useful ligands and/or modulators of DPPI, including efficient, stabile and non-toxic inhibitors of DPPI. The present invention also provides the means for designing DPPI mutants with optimised properties and/or with other specific characteristics and also for the modelling of the structure of different variants of DPPI, including but not limited to DPPI from different species, a DPPI mutant and DPPI or DPPI mutant complexed with specific ligands.

First of all, the present invention provides a crystal containing a rat DPPI protein that effectively diffracts X-rays and thereby allows the determination of the atomic co-ordinates of a protein to a resolution greater than 5.0 Ångströms. In a preferred embodiment of this type, the crystal effectively diffracts X-rays for the determination of the atomic co-ordinates of said protein to a resolution greater than 3.0 Ångströms, and in an even more preferred embodiment, the crystal effectively diffracts X-rays for the determination of the atomic co-ordinates of a DPPI protein to a resolution of at least 2.0 Ångströms.

Furthermore, the present invention provides the crystal structural co-ordinates for human DPPI.

In one embodiment of the invention, the crystal comprises the amino acid sequence of a protein being at least 75%, such as 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to rat DPPI, as shown in FIG. 1, including DPPI from different species, such as human or mouse DPPI. In another embodiment of the invention, even a crystal comprising an amino acid sequence of a protein being as little as at least 37% overall identical to rat DPPI are embodied.

The rat DPPI amino acid sequence shown in FIG. 1 is identical to the one shown in SEQ. ID. NO. 1.

Preferably, a crystal comprises an amino acid sequence of a protein having a polypeptide sequence which shares at least 37% (more preferably at least 45%, even more preferably at least 55%, and most preferably at least 65%) amino acid sequence identity to the amino acid sequence of rat DPPI (FIG. 1) and at least 50% (more preferably at least 60%, even more preferably at least 70%, and most preferably at least 80%) amino acid sequence identity to the catalytic domain of human DPPI, as determined by pair-wise sequence alignment using the computer program Clustal W 1.8 (Thompson et al. (1994) Nucleic Acids Res. 22, 4673-4680).

The crystal ideally comprises the amino acids of proteins that are homologous to rat DPPI and/or display a functional homology to rat DPPI, such as an aminopeptidase activity and/or a transferase activity. In a preferred embodiment of the invention, the crystal comprises a protein with an amino acid sequence as shown in FIG. 1.

The present invention provides a crystal of a DPPI-like enzyme wherein the space group is P6₄₂₂ and the unit cell dimensions are a=166.24 Å, b=166.24 Å, c=80.48 Å with α=β=90° and γ=120°. The rat DPPI structure disclosed in the present invention is listed in Table 2 and provides new and surprising insight into the structural arrangement of DPPI. The protein was crystallised as a tetramer in accordance with the oligomeric structure of the enzyme in vivo.

The present invention further provides a crystal of a DPPI-like protein having structural elements comprising subunits that are assembled in a ring-like structure with the residual pro-parts and catalytic domains of neighbouring subunits being assembled head-to-tail so that each kind of domain points upwards and downwards, alternately, and the active sites point away from the centre of the ring (FIG. 3). The catalytic domain of rat DPPI is herein shown to have a similar fold to papain (FIGS. 4 and 5). Residues 1-119 form a well-defined beta-barrel domain with little or no alpha helical structure.

The present invention hereby provides a crystal structure model of a DPPI-like protein,

wherein the residual pro-part domain is located relative to the catalytic domain blocking the extreme end of the unprimed active site cleft. Most significantly, the N-terminus of the residual pro-part projects further towards the catalytic residues and the free amino group of the conserved Asp1 is held in position by a hydrogen bond to the backbone oxygen atom of Asp274. This arrangement provides a negative charge, located on the side chain of Asp1, in a fixed position within the active site cleft. The delocalised negative charge that this residue carries under physiological conditions on its OD1 and OD2 oxygen atoms is localised about 7.4 and 8.7 Å from the sulphur atom of the catalytic Cys233 residue. Thus, the present invention provides proof that the protonated N-termini of peptide substrates form a salt bridge to the negative charge on the side chain of Asp1. Furthermore, the position of the N-terminal Asp1 residue is shown to be fixed by a hydrogen bond between the free amino group of this residue (hydrogen bond donor) and the backbone carbonyl oxygen of Asp274 (hydrogen bond acceptor).

The present invention thus elucidates a surprising and novel principle for substrate binding that can be used in constructing models for other substrate binding peptides. The donation of a negative charge in the active site cleft of a cysteine peptidase by the side chain of the N-terminal residue of the residual pro-part is a novel structural feature not previously observed.

In the crystal structure of the present invention, a wide and deep pocket is located between Asp1 and Cys233, which may accommodate the side chains of one or both of the two most N-terminal substrate residues. In addition to Asp1 and Cys233, this pocket is defined by residual pro-part, heavy chain and light chain residues including, but not limited to, Tyr64, Gly231, Ser232, Tyr234, Ala237, Asp274, Gly275, Gly276, Phe277, Pro278, Thr378, Asn379, His 380, Ala381.

The active sites in DPPI proteins from different species can be expected to be structurally very similar. Therefore, the present invention provides a very good and usable model for the active sites of most mammalian DPPI, including but not limiting to that of human DPPI.

The present invention also relates to a method for growing a crystal of a DPPI-like protein. This method comprises obtaining a stock solution containing 1.5 mg/ml of a DPPI-like protein in 25 mM sodium phosphate pH 7.0, 150 mM NaCl, 1 mM ethylene diamine triacetate (EDTA), 2 mM cysteamine and 50% glycerol, dialysing a portion of the stock solution against 20 mM bis-tris-HCl pH 7.0, 150 mM NaCl, 2 mM dithiothreitol (DTT), 2 mM EDTA and employing the hanging drop vapour diffusion technique with 0.8 ml reservoir solution and drops containing 2 μl protein solution and 2 μl reservoir solution in conditions employing (0.1 M Tris pH 8.5, 2.0 M (NH₄)₂SO₄). In a preferred embodiment, the method of the present invention will thus result in the formation of star-shaped crystals or alternatively in the formation of box-shaped crystals.

In a specially preferred embodiment, an optimum for a box shaped crystal form is obtained by using reservoir solution containing 0.1 M bis-tris propane pH 7.5, 0.15 M calcium acetate and 10% PEG 8000. Drops are optimally set up with equal volumes of reservoir solution and protein solution wherein the protein concentration is 12 mg/ml.

In another, equally preferred embodiment, optimal crystallisation conditions for a star-shaped crystal form are provided at 1.4 M (NH₄)₂SO₄ and 0.1 M bis-tris propane pH 7.5.

The present invention further provides methods of screening drugs or compositions or polypeptides that either enhance or inhibit DPPI enzymatic activity. A concept based on inhibition of DPPI for therapeutic intervention against the above mentioned mast cell, neutrophils and cytotoxic lymphocytes proteinase mediated diseases is included.

As DPPI is a dipeptidyl peptidase with a unique specificity, it is potentially more simple to design specific and effective DPPI inhibitors, which do not cross-react with proteinases of the same family than to develop tryptase, chymase, granzyme A, B and K, elastase and cathepsin G inhibitors. Therefore, the present invention will provide the means for designing a specific and effective therapeutic inhibitor against mast cell, neutrophils and cytotoxic lymphocytes proteinase mediated diseases.

Due to the lower cellular levels of DPPI compared to the levels of tryptase, chymase, granzyme A, B and K, elastase and cathepsin G, inhibition of DPPI activity is also presumed to be more easily accomplished.

The present invention will further make it possible to design DPPI inhibitor prodrugs that are resorbed as inactive inhibitors and subsequently activated to their active forms by either tryptase, chymase, granzyme A, B and K, elastase and cathepsin G, specifically at the site of their release, due to activation of mast cell, neutrophils and cytotoxic lymphocytes at the site of inflammation or immunoreaction.

Furthermore, DPPI has been assigned an important role in the life circle of several species of blood flukes of the genus Scistosoma, which as adult live and lay eggs in the blood vessels of the intestines, bladder and other organs. These Scistosoma blood flukes cause scistosomiasis, which is considered the most important of the human helminthiases in terms of morbidity and mortality. Scistosomes are obligate blood feeders and haemoglobin from the host blood is essential for Scistosoma parasite development, growth and reproduction. Haemoglobin released from the erythrocytes of the host is catabolyzed by the Scistosoma to dipeptides and free amino acid and then incorporated into Scistosoma proteins. The enzymes that participate in the pathway for degradation of haemoglobin into amino acid components useful for the Scistosoma parasite are not fully known. DPPI, however, is believed to play a key-role in degrading small peptides, generated from haemoglobin by endopeptidases, to dipeptides, which then can be taken up by simple diffusion or by active transport via an oligopeptide transporter system. Thus DPPI is pointed out as an important target enzyme for therapeutic intervention against Scistosoma blood flukes scistosomiasis, by using a DPPI-inhibition concept similar to the above mentioned concept for therapeutic intervention against mast cell, neutrophils and cytotoxic lymphocytes proteinase mediated diseases.

Thus, the present invention provides a method for using the crystals of the present invention or the structural data obtained from these crystals for drug and/or inhibitor screening assays. In one such embodiment the method comprises selecting a potential drug by performing rational drug design with the three-dimensional structure determined from the crystal. The selecting is preferably performed in conjunction with computer modelling. The potential drug or inhibitor is contacted with a DPPI-like protein or a domain of a DPPI-like protein and the binding of the potential drug or inhibitor with this domain is detected. A drug is selected which binds to said domain of a DPPI-like protein or an inhibitor, which successfully inhibits the enzymatic activity of DPPI.

In a preferred embodiment of the present invention, the method further comprises growing a supplemental crystal containing a protein-co-complex or a protein-inhibitor complex formed between the DPPI-like protein and the second or third component of such a complex. The crystal effectively diffracts X-rays, allowing the determination of the co-ordinates of the complex to a resolution of greater than 3.0 Ångströms and more preferably still, to a resolution greater than 2.0 Ångströms. The three-dimensional structure of the supplemental crystallised protein is then determined with molecular replacement analysis.

A drug or an inhibitor is selected by performing rational drug design with the three-dimensional structure determined for the supplement crystal. The selecting is preferably performed in conjunction with computer modelling.

In addition, in order to fully exploit the potential of the combined processes of using purification tags for purification of recombinant proteins and DPPI for cleavage of the purification tag generating the desired N-terminal in the target protein (the DPPI/tag strategy), the present invention further provides the means to alter the chemical, physical and enzymatic properties of DPPI to be able to use the enzyme in different conditions, thus making the DPPI/tag strategy more efficient, flexible and/or even more economic feasible. These changes could include e.g. increase in the thermostability, increase in the stability towards chaotropic agents and detergents, increase in the stability at alkaline pH, changes in certain amino acids residues for targeted chemical modifications, changes in the catalytic efficiency (K_{cat}/K_{M}) or changes to the catalytic specificity. In addition, it could be desirable to alter the oligomeric structure of DPPI or to enhance the intramolecular interactions between the DPPI subunits or domains. Furthermore, the knowledge provided in the present invention of the crystal structure co-ordinates and atomic details of DPPI will enable the design of efficient and specific immunoassays for the important and necessary tracing of DPPI at different stages during protein purification processes based on the DPPI/tag strategy.

Regarding the transferase activity of DPPI, knowledge of the crystal structure co-ordinates and atomic details of DPPI, elucidated in the present invention, will enable the design of mutants of DPPI with different ratios between aminopeptidase and transferase activity and reduced levels of substrate restrictions, making them suitable for effective enzymatic synthesis or semisynthesis of peptides and proteins. Because of a simple overall design and the use of non-toxic and efficient enzymes, the use of DPPI mutants, with optimised properties with respect to transpeptidase reactions, holds promises for use in large-scale productions of pharmaceutical protein and peptide products.

The present invention thus relates to the crystal structure, atomic co-ordinates and structural models of DPPI, of forms of DPPI which contain at least a part of the catalytic domain and of mutants of any of these enzyme forms or partial enzyme forms. The present invention also provides a method for designing chemical entities capable of interacting with DPPI, with propPPI or with any naturally existing form of partially processed propPPI. Furthermore, the present invention provides the structural basis for the design of mutant forms of DPPI with altered characteristics and functionality.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C §112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIG. 1. Amino acid sequence of rat DPPI (SEQ ID NO: 1) and nucleic acid sequence of rat DPPI (SEQ ID NO: 2).

FIG. 2. Clustal W allignment of amino acid sequences of propPPI (DPPI proenzyme) from different species SEQ ID NOs: 1 and 3-11). Using rat propPPI numbering the four sequence regions are:residuel pro-part (residues 1-119), activation peptide (residues 120-205), heavy chain (residues 206-369) and light chain (residues 370-438). Minor differences have been observed.

FIG. 3. The rat DPPI tetramer with each subunit oriented with either the residual pro-part in the front as in FIG. 5: monomer 1 BW.jpg (upper right and lower left subunits) or with the catalytic domain in the front (upper left and lower right subunits).

FIG. 4. Schematic presentation of a rat DPPI subunit (upper molecule) and of papain (lower molecule). One subunit of rat DPPI is clearly formed by two domains (the residual pro-part domain (residues D1-M118) and the catalytic domain (residues L204-H365 and P371-L438)) of which the latter shows structural homology to papain.

FIG. 5. Rat DPPI monomer with the beta-barrel residual pro-part domain in the front and catalytic domain in the back.

FIG. 6. Cathepsin C crystal grown from 0.15 M Bis-tris propane, pH 7.5 and 10% PEG 8000.

FIG. 7. The cathepsin C crystal form used to determine the molecular structure of the enzyme. This is a single crystal. Diameter varied between 0.5 and 1 mm, thickness at center between 0.1 and 0.4 mm. Crystals were grown from 0.1 M Bis-tris propane, pH 7.5 and 1.4M (NH₄)₂SO₄.

FIG. 8. Results from transferase activity assay of wild tye and Asp274 to Gln274 and of Asn226:Ser229 to Gln226:Asn229 mutants of rat DPPI

FIG. 9: Shows a model of the structure of a monomer of human DPPI made based on the structural data of rat DPPI. The crystal structure of rat DPPI refined to a resolution of 2.4 Å was used as a template for comparative modeling of the human enzyme. The amino acid sequences of the rat and human enzymes were aligned using the program Clustal W. The sequence identity is .about.80% for the full length sequences of the rat and human enzymes. Comparative modeling of the human enzyme was performed using the program Modeller (A. SalI and T. L. Blundell (1993) Comparative protein modelling by satisfaction of spatial restraints. J. Mol. Biol. 234, 779-815). The positional root mean square deviation of superimposed CA atoms in the rat and the modelled human structure was determined to 0.2 Å. using the program DALI (L. Holm and C Sander (1996) Mapping the protein universe. Science 273, 595-602).

FIG. 10: Tetrahedral structure of human DPPI

a) Molecular surface of tetrahedral structure of DPPI. Surfaces of papain-like domains and residual propart domains are shown. The view is along two active sites towards the residual propart domain hairpin loop (Lys 82-Tyr 93) building a wall behind the active site cleft and five N-terminal residues shown in orange. The left and right molecules are shown from the back towards the residual propart domain. The molecular surface was generated with GRASP (Nicholls et al., 1991), the figure was prepared in MAIN (Turk, 1992) and rendered with RENDER (Merrilt and Bacon, 1997).

b) DPPI dimer. Head-to-tail arrangement of two pairs of papain-like and residual propart domains. The view is from the inside of the tetramer along the dimer twofold. The figure was created with RIBBONS (Carson, 1991).

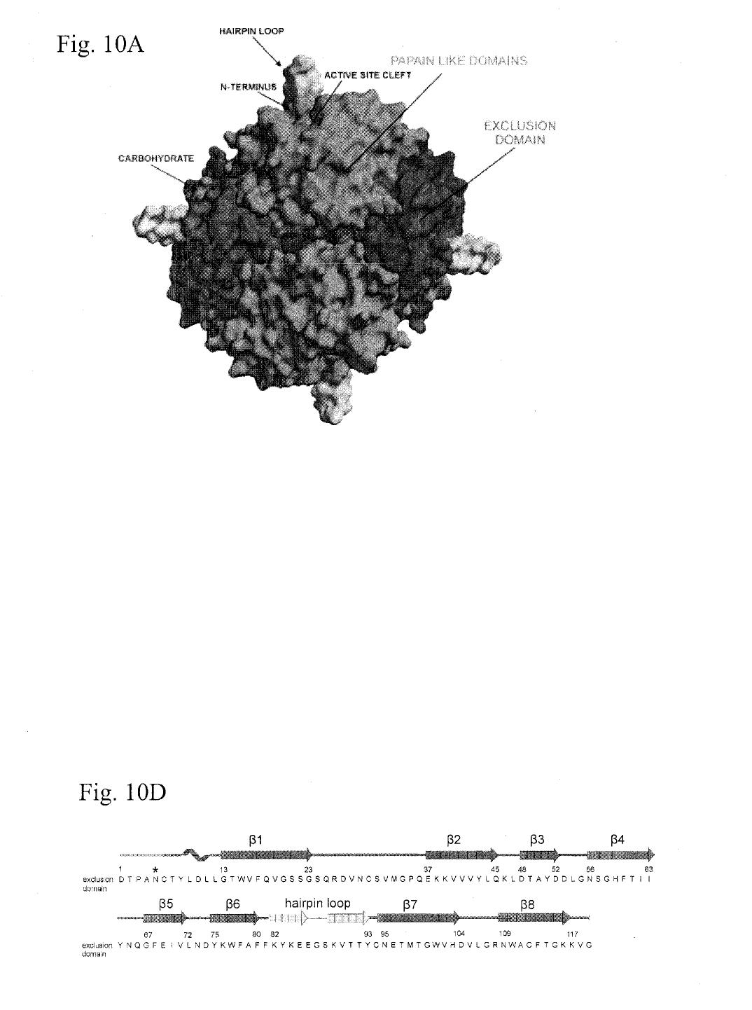

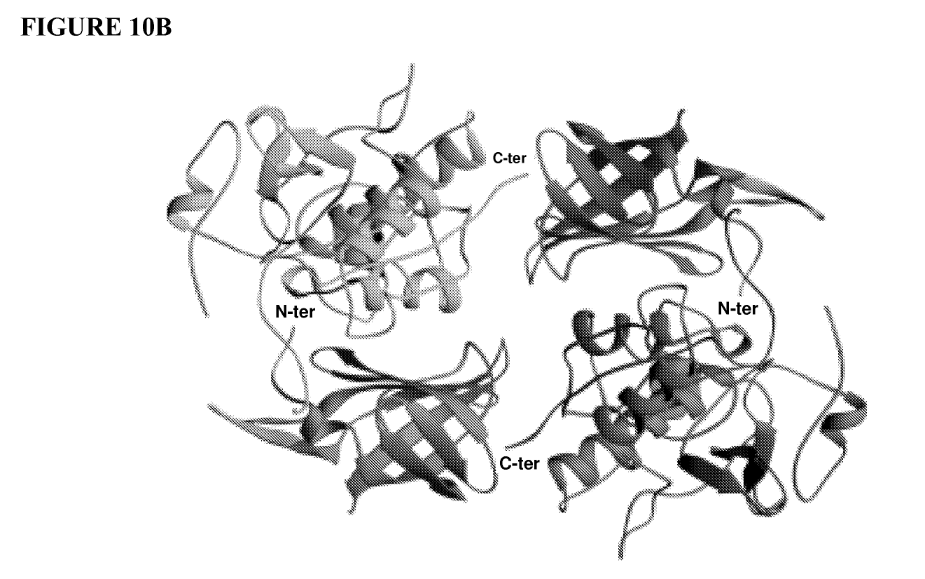

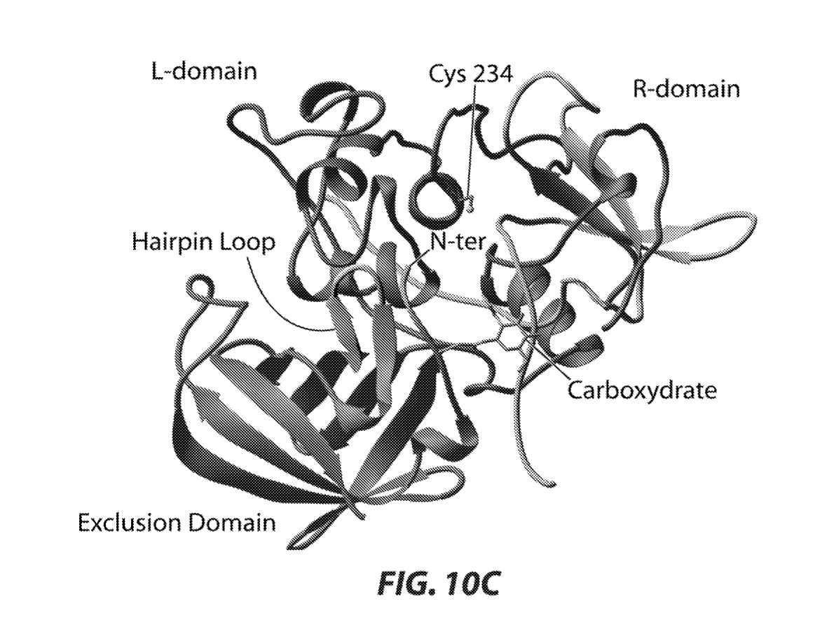

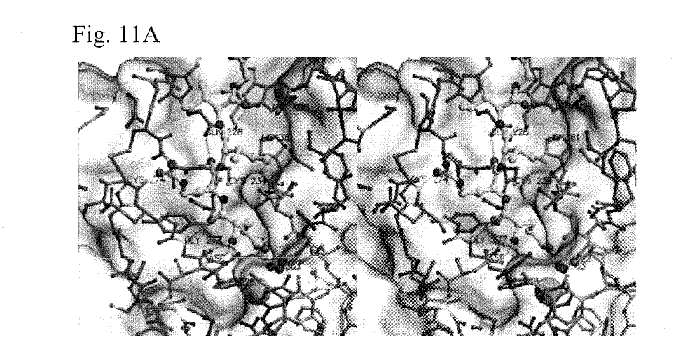

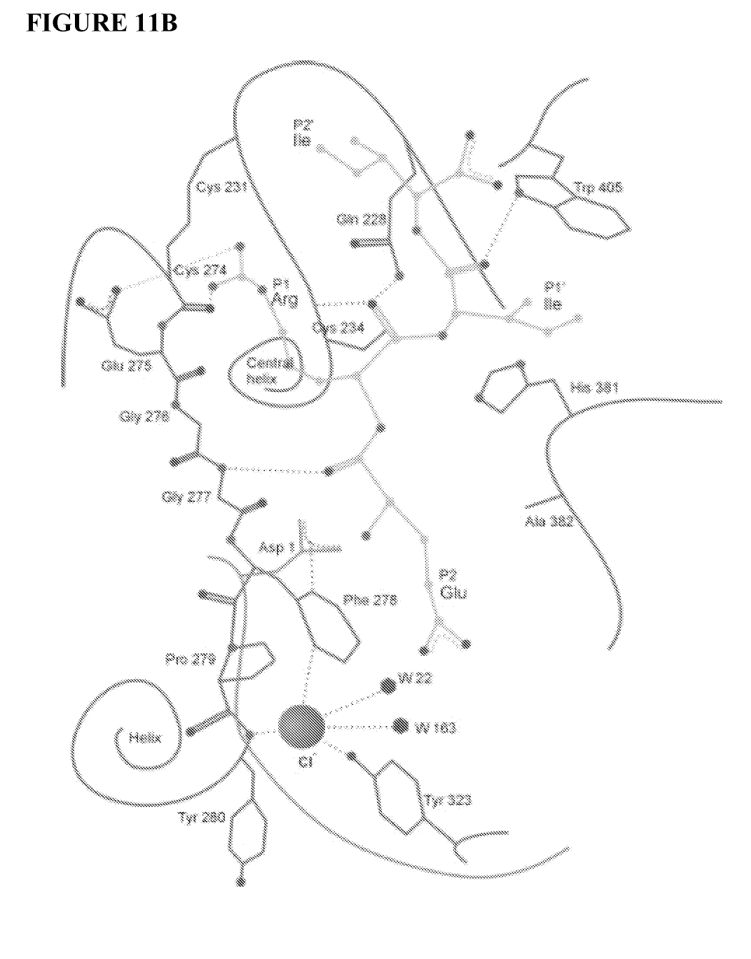

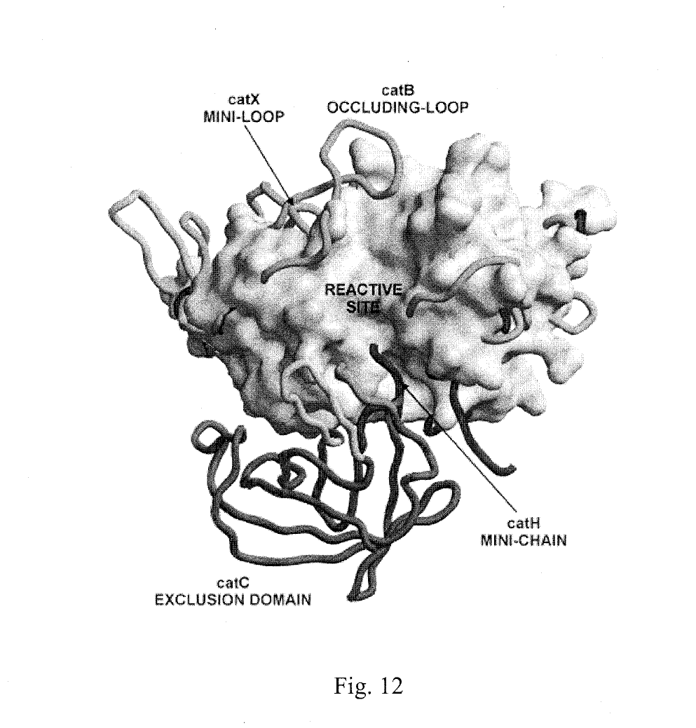

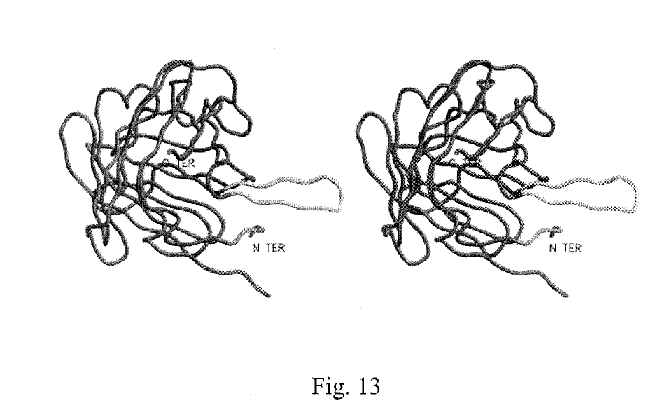

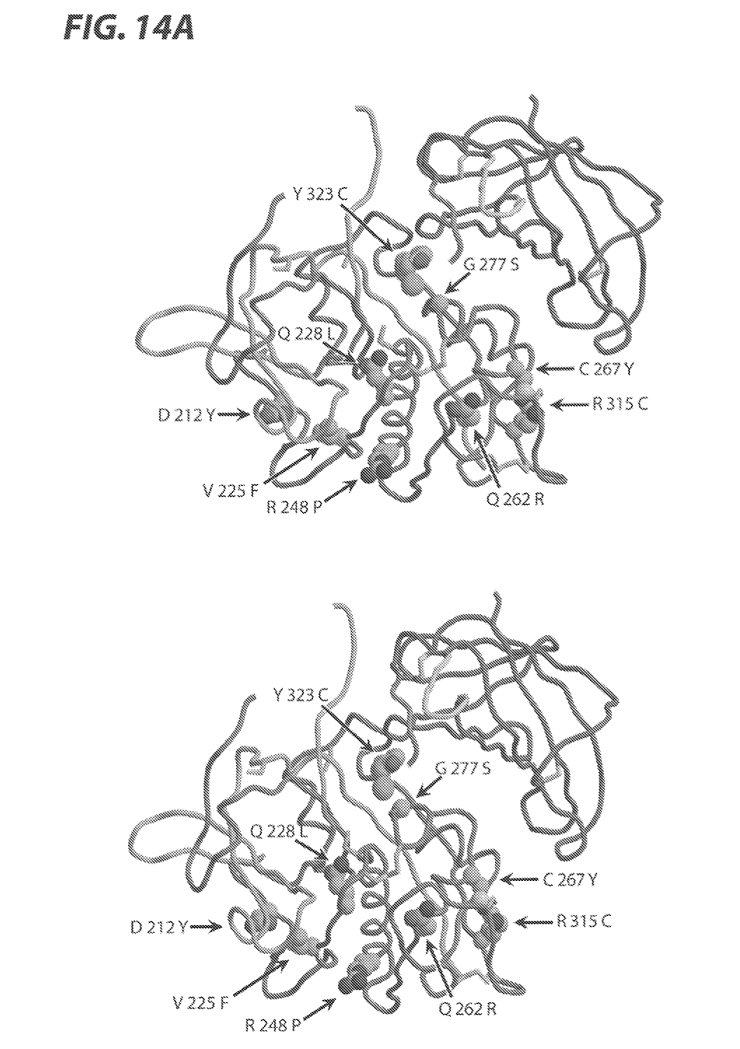

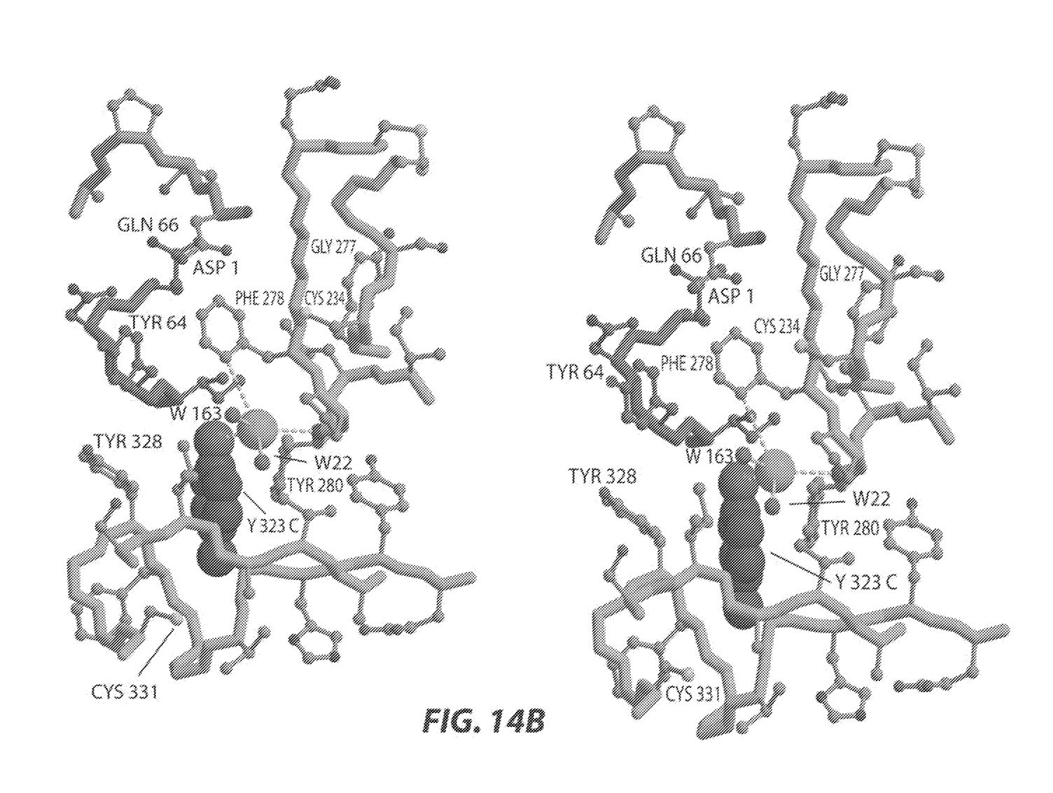

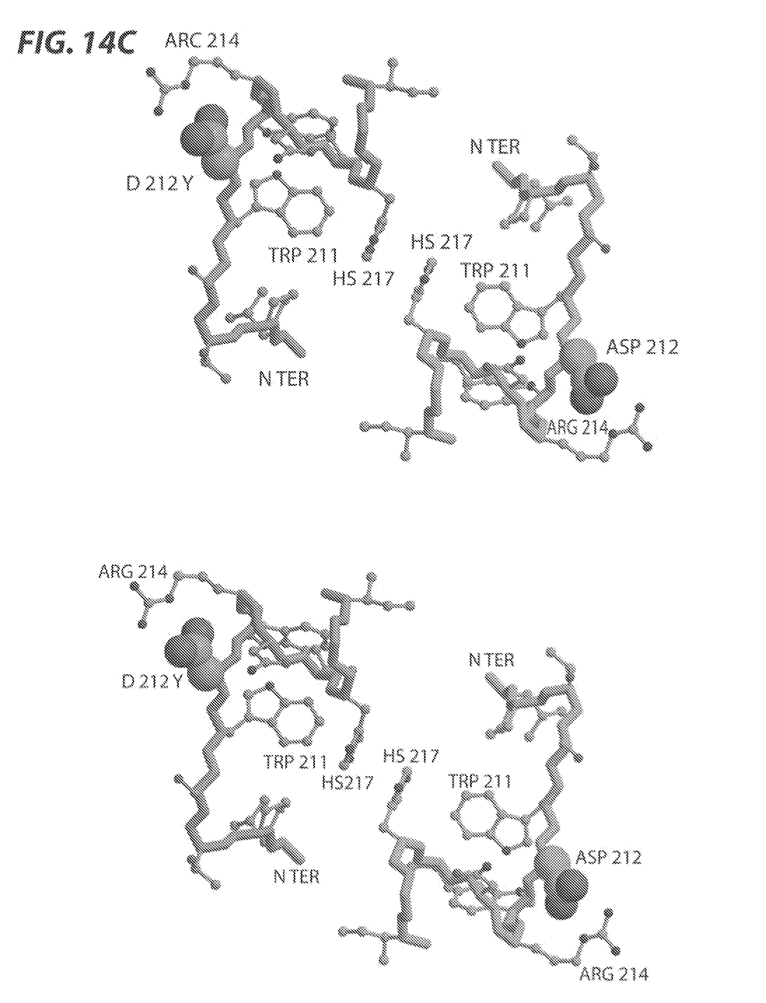

c) Ribbon plot of the functional monomer of DPPI (SEQ ID NO: 12). The view shows the structure from the top, down the central alpha helix. It is perpendicular to the view used in FIG. 10a. The side chain of catalytic Cys 234 and disulfides are shown with yellow sticks. The figure was created with RIBBONS (Carson, 1991).

d) sequence of residual propart domain with its secondary structure assignment.

FIG. 11: Active site cleft of human DPPI with a bound model of the N-terminal sequence ERIIGG from the biological substrate, granzyme A.

a) Stereo view: Covalent bonds of papain-like domains and residual propart domain are shown. Covalent bonds of substrate model are shown. To them corresponding carbon atoms are shown as balls using the covalent bond scheme. Chloride ions is shown as a large sphere. Oxygen, nitrogen and sulphur atoms are shown as grey spheres. The residues relevant for substrate binding are marked and hydrogen bonds are shown as white broken lines. The molecular surface was generated with GRASP (Nicholls et al., 1991), the figure was prepared in MAIN (Turk, 1992) and rendered with RENDER (Merritt and Bacon, 1997).

b) Schematic presentation. The same codes are used as in FIG. 11a.

FIG. 12: Features of papain-like exopeptidases.

A view towards the active site clefts of superimposed papain-like proteases. The underlying molecular surface of cathepsin L, shown in white, is used to demonstrate an endopeptidase active site cleft, which is blocked by features of the exopeptidase structures. Chain traces of cathepsins B, X, H are shown. Bleomycin hydrolase chain trace is not shown for clarity reasons although its C-terminal residues superimpose almost perfectly to the C-terminal residues of cathepsin H mini-chain.

FIG. 13: Superposition of erwinia chrysanthemi metallo protease inhibitor on the residual propart domain.

The figure was prepared with MAIN (Turk, 1992) and rendered with RENDER (Merritt and Bacon, 1997).

FIG. 14: Regions with missense mutations resulting in genetic diseases.

The figures were prepared with MAIN (Turk, 1992) and rendered with RENDER (Merritt and Bacon, 1997).

a) Missense mutations overview. Mutated residues are marked with their sequence IDs and residue names in one letter code. The catalytic cysteine is also marked.

b) Y323C mutant with chloride ion coordination. A side view towards the S2 binding pocket containing the chloride ion and its coordination with the active site residues Asp 1 and Cys 234 at the top. The main chain bonds are thicker. Oxygens of the main chain carbonyls are omitted for clarity. The chloride ion is a large ball and the small balls adjacent to it are solvent molecules. Chloride coordination is shown with disconnected sticks. Relevant residues are marked with their sequence IDs and residue names.

c) D212Y mutant: View along a molecular twofold. Asp 212 side chain atoms are pronounced as bigger balls.

### DETAILED DESCRIPTION

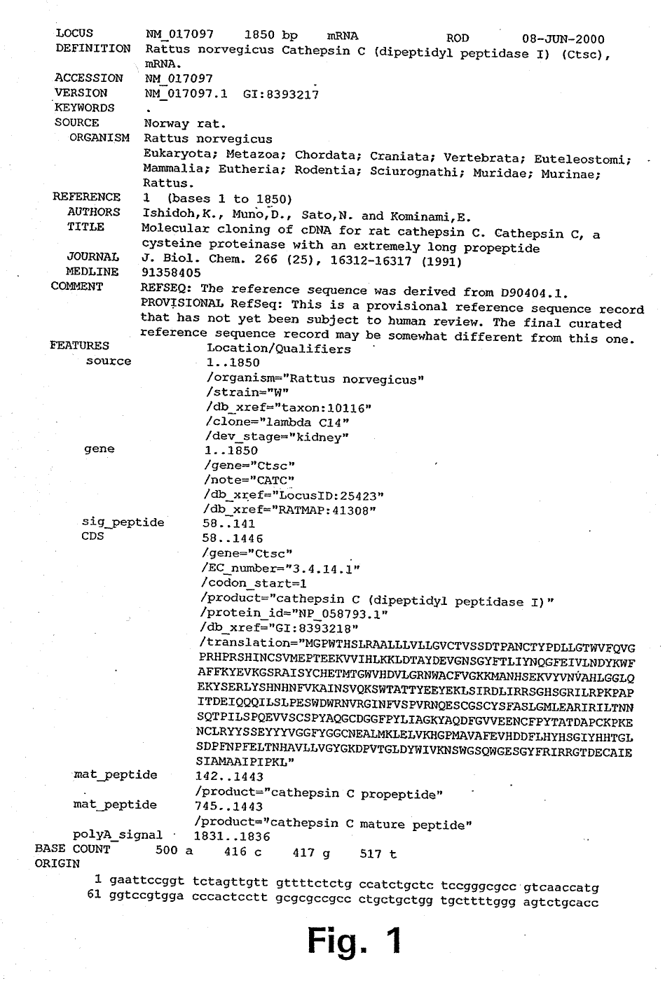

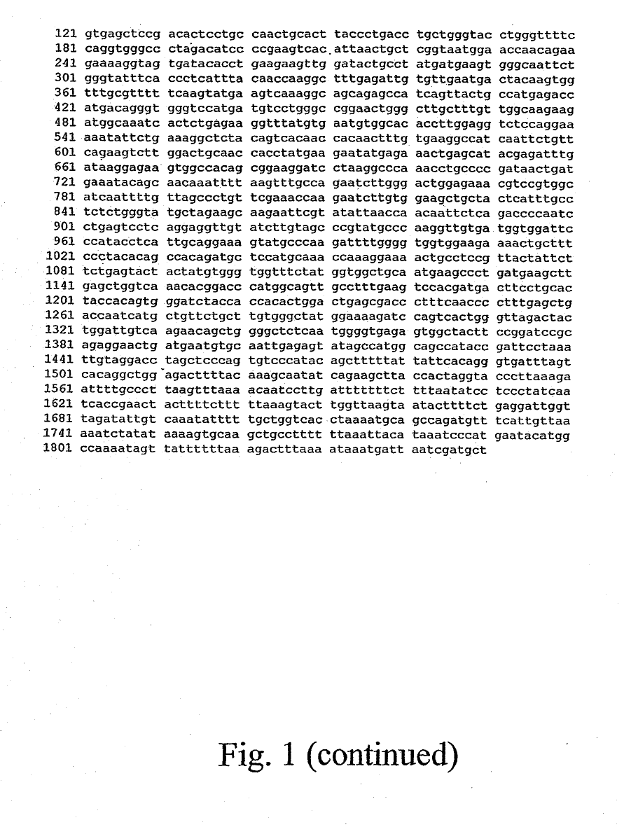

The term “DPPI” refers to dipeptidyl peptidase I also known as DPPI, DAPI, dipeptidyl aminopeptidase I, cathepsin C, cathepsin J, dipeptidyl transferase, dipeptidyl arylamidase and glucagon degrading enzyme. The term also refers to any polypeptide which shares at least 37% amino acid sequence identity to the amino acid sequence of rat DPPI (FIG. 1) and at least 50% amino acid sequence identity to the catalytic domain of human DPPI as determined by pair-wise sequence alignment using the computer program Clustal W 1.8 (Thompson et al. (1994) Nucleic Acids Res. 22, 4673-4680). The enzyme may be of mammalian, avian or insect origin. Alternatively, the enzymes may be obtained by expressing the genes or cDNAs encoding the enzymes or enzyme mutants or enzyme fusions or hybrids hereof in a recombinant system.

The term “pro-DPPI” refers to the single chain proenzyme form of dipeptidyl peptidase I. The term also refers to any polypeptide which shares at least 37% amino acid sequence identity to the amino acid sequence of rat DPPI (FIG. 1) and at least 50% amino acid sequence identity to the catalytic domain of human DPPI as determined by pair-wise sequence alignment using the computer program Clustal W 1.8.

“DPPI-like protein” are proteins composed of one or more polypeptide chains which has an overall amino acid sequence that is at least 30% identical to the amino acid sequence of mature rat DPPI according to SEQ. ID. NO. 1 and which includes a sequence that is at least 30% identical to the residual pro-part domain of rat DPPI.

“Equivalent back bone atoms” following Clustal W 1.8 alignment of two or more homologous amino acid sequences, the equivalent back bone atoms can be identified as those polypeptide back bone nitrogen, alpha-carbon and carbonyl carbon atoms of two or more amino acid residues that are aligned in the same position. For example, in an alignment of two polypeptide sequences, the atom which is equivalent to a back bone nitrogen atom in one residue is the back bone nitrogen atom in the residue in the other sequence which is aligned in the same position. The atoms in residues that are not aligned, e.g. because of a gap in the other sequence or because of different sequence lengths, do not have equivalent back bone atoms.

The term “structural alignment” refers to the superpositioning of related protein structures in three-dimensional space. This is preferably done using specialised computer software. The optimum structural alignment of two structures is generally characterised by having the global minimum root-mean-square deviation in three-dimensional space between equivalent backbone atoms. Optionally, more atoms may be included in the structural alignment, including side chain atoms.

The term “processed” refers to a molecule that has been subjected to a modification, changing it from one form to another. More specifically, the term “processed” refers to a form of pro-DPPI which has been subjected to at least one post-translational chain cleavage (per subunit) in addition to any cleavage resulting in the excision of a signal peptide.

The term “mature” refers to pro-DPPI following native like processing, i.e. processing similar to the processing natural pro-DPPI in vivo. The mature product, DPPI, contains at least about 80% of the residual pro-part, 90% of the heavy and light chain residues and less than 10% of the activation peptide residues.

The term “heavy chain” refers to the major peptide in the catalytic domain of DPPI. In human DPPI, the heavy chain constitutes the proenzyme residues 200-370 or more specifically residues 204-370 or residues 206-370 or even more specifically residues 207-370.

The term “light chain” refers to the minor peptide in the catalytic domain of DPPI. In human DPPI, the light chain constitutes the proenzyme residues 371-439.

The term “proregion” refers to the region N-terminal of the catalytic domain region of pro-DPPI. In human pro-DPPI, the proregion constitutes residues 1-206 or residues 1-205 or residues 1-203 or residues 1-199.

The term “activation peptide” refers to the part of the proregion in pro-DPPI, which is excised in the mature form of the enzyme. In human DPPI, the activation peptide constitutes residues 120-206 but may also constitute residues 120-199, 120-203, 120-205, or 120-206 or residues 134-199, 134-203, 134-205, or 134-206. The N-terminal and C-terminal residues are not confirmed and may vary. The activation peptide of pro-DPPI is thought to be homologous to the propeptides of cathepsins L and S.

The term “residual pro-part” refers to the part of the proregion in pro-DPPI, which is not excised in the mature form of the enzyme.

The term “catalytic domain” refers to the structural unit, which is formed by the heavy chain and light chain in mature DPPI. The structure of the catalytic domain is presumed to be homologous to the structures of mature papain and cathepsins L, S, B etC

The term “inhibitors” refers to chemical compounds, peptides and polypeptides that inhibit the activity of one or more enzymes by binding covalently or non-covalently to the enzyme(s), typically at or close to the active site.

The term “protease inhibitors” refers to chemical compounds, peptides and polypeptides that inhibit the activity of one or more proteolytic enzymes. By selecting a specific protease inhibitor or kind of protease inhibitor(s), it is often possible to specifically inhibit the activity of one or more proteases or types of proteases; E-64 and cystatins (e.g. human cystatin C) are relatively non-specific covalent and non-covalent cysteine proteinase inhibitors, respectively. EDTA inhibits Ca²⁺ and Zn²⁺ dependent metalloproteases and PMSF inhibits serine proteases. In contrast, TLCK and TPCK are both inhibitors of serine and some cysteine proteases but only TLCK inhibits trypsin and only TPCK inhibits chymotrypsin.

The term “mutant” refers to a polypeptide, which is obtained by replacing or adding or deleting at least one amino acid residue in a native pro-DPPI with a different amino acid residue. Mutation can be accomplished by adding and/or deleting and/or replacing one or more residues in any position of the polypeptide corresponding to DPPI.

The term “homologue” refers to any polypeptide, which shares at least 25% amino acid sequence identity to the reference protein as determined by pair-wise sequence alignment using the computer program Clustal W 1.8 (Thompson et al. (1994) Nucleic Acids Res. 22, 4673-4680).

The term “subunit” refers to a part of DPPI. Native DPPI consists of four subunits formed by association of four modified translation products.

The term “preparative scale” refers to expression and/or isolation of a protein in an amount larger than 0.1 mg.

The term “active site” refers to the cavity in each DPPI subunit into which the substrate binds and wherein the catalytic and substrate binding residues are located.

The term “catalytic residues” refers to the cysteine and histidine residues in each DPPI subunit, which participate in the catalytic reaction. In human pro-DPPI, the catalytic residues are cysteine 234 and histidine 381.

The term “substrate binding residues” refers to any DPPI residues that may participate in binding of a substrate. Substrates may interact with both the side chain and main chain atoms of DPPI residues.

When used to describe a preparation of a protein or polypeptide, the terms “pure” or “substantially pure” refer to a preparation wherein at least 80% (w/w) of all protein material in said preparation is said protein.

In descriptions of homology between amino acid sequences, the term “identical” refers to amino acid residues of the same kind that are matched following pairwise Clustal W 1.8 alignment (Thompson et al. (1994) Nucleic Acids Res. 22, 4673-4680) of two known polypeptide sequences using a sequence alignment method, such as ClustalW2. ClustalW2 is available at the website of the European Bioinformatics Institute website. The program was run using the following parameters: scoring matrix: blosum; opening gap penalty: 1. The percentage of amino acid sequence identity between such two known polypeptide sequences is determined as the percentage of matched residues that are identical relative to the total number of matched residues.

“Identity” as known in the art, is a relationship between two or more polypeptide sequences or two or more polynucleotide sequences, as determined by comparing the sequences. In the art, “degree of sequence identity” or “percentage of sequence identity” also means the degree of sequence relatedness between polypeptide or polynucleotide sequences, as the case may be, as determined by the match between strings of such sequences following Clustal W 1.78 alignment. “Identity” and “similarity” can readily be calculated by known methods.

The term “naturally occurring amino acids” refers to the 20 amino acid that are encoded by nucleotide sequences; alanine (Ala, A), cysteine (Cys, C), aspartate (Asp, D), glutamate (Glu, E), phenylalanine (Phe, F), glycine (Gly, G), histidine (His, H), isoleucine (Ile, I), lysine (Lys, K), leucine (Leu, L), methionine (Met, M), asparagine (Asn, N), proline (Pro, P), glutamine (Gln, Q), arginine (Arg, R), serine (Ser, S), threonine (Thr, T), valine (Val, V), tryptophane (Trp, W) and tyrosine (Tyr, Y). The three-letter and one-letter abbreviations are shown in brackets. Two cysteines may form a disulfide bond between their gamma-sulphur atoms.

The term “unnaturally occurring amino acids” includes amino acids that are not listed as naturally occurring amino acids. Unnaturally occurring amino acids may originate from chemical synthesis or from modification (e.g. oxidation, phosphorylation, glycosylation) in vivo or in vitro of naturally occurring amino acids.

The term “substrate” refers to a compound that reacts with an enzyme. Enzymes can catalyse a specific reaction on a specific substrate. For example, DPPI can in general excise an N-terminal dipeptide from a peptide or peptide-like molecule except if the N-terminal residue is positively charged and/or if the cleavage site is on either side of a proline residue. Other factors, such as steric hindrance, oxidation of the substrate, modification of the enzyme or presence of unnaturally occurring amino acids, may also prevent DPPI's catalytic activity.

The term “specific activity” refers to the level of enzymatic activity of a given amount of enzyme measured under a defined set of conditions.

The term “crystal” refers to a polypeptide in crystalline form. The term “crystal” includes native crystals, derivative crystals and co-crystals, as described herein.

The term “native crystal” refers to a crystal wherein the polypeptide is substantially pure.

The term “derivative crystal” refers to a crystal wherein the polypeptide is in covalent association with one or more heavy atoms.

The term “co-crystal” refers to a crystal of a co-complex.

The term “co-complex” refers to a polypeptide in association with one or more compounds.

The term “accessory binding site” refers to sites on the surface of DPPI other than the substrate binding site that are suitable for binding of ligands.

“Crystal structure” in the context of the present application refers to the mutual arrangement of the atoms, molecules, or ions that are packed together in a regular way to form a crystal.

“Atomic co-ordinates” is herein used to describe a set of numbers that specifies the position of an atom in a crystal structure with respect to the axial directions of the unit cell of the crystal. Co-ordinates are generally expressed as the dimensionless quantities x, y, z (fractions of unit-cell edges). “Structure co-ordinates” refers to a data set that defines the three dimensional structure of a molecules or molecules. Structure co-ordinates can be slightly modified and still render nearly identical structures. A measure of a unique set of structural co-ordinates is the root-mean-square deviation of the resulting structure. Structural co-ordinates that render three dimensional structures that deviate from one another by a root-mean-square deviation by less than 1.5 Å may be viewed by a person skilled in the art as identical. Hence, the structure co-ordinates set forth in Table 2 are not limited to the values defined therein.

The term “heavy atom derivative” refers to a crystal of a polypeptide where the polypeptide is in association with one or more heavy atoms.

The terms “heavy atom” and “heavy metal atom” refer to an atom that is a transition element, a lanthanide metal (includes atom numbers 57-71, inclusive) or an actinide metal (includes atom numbers 89-103, inclusive).

The term “unit cell” refers to the smallest and simplest volume element of a crystal that is completely representative of the unit of pattern of the crystal. The dimensions of the unit cell are defined by six numbers: dimensions a, b and c and angles alpha (α), beta (β) and gamma (γ).

The term “multiple isomorphous replacement” (MIR) refers to a method of using heavy atom derivative crystals to obtain the phase information necessary to elucidate the three dimensional structure of a native crystal. The phrase “heavy atom derivatization” is synonymous with “multiple isomorphous replacement”.

The term “molecular replacement” refers to the method of calculating initial phases for a new crystal whose atomic structure co-ordinates are unknown. The method involves orienting and positioning a molecule, for which the structure co-ordinates are known and which is presumed to have a three dimensional structure similar to that of the crystallised molecule, within the unit cell of the new crystal so as to best account for the observed diffraction pattern of the new crystal. Phases are then calculated from this model and combined with the observed amplitudes to provide an approximate Fourier synthesis of the structure of the molecules comprising the new crystal. This, in turn, is subject to any of several methods of refinement to provide a final, accurate set of structure co-ordinates for the new crystal.

The term “prodrug” refers to an agent that is converted to the parent drug in vivo. A prodrug may be more favourable if it e.g. is bioavailable by oral administration and the parent drug is not or if it has more favourable pharmacokinetic and/or solubility properties.

Description of the Rat DPPI Structure

The rat DPPI structure disclosed in the present invention (Table 2) has revealed several structural features not present in any known structure of a papain family peptidase. The electron density defines the spatial arrangement of the residual pro-part residues Asp1 to Met118, heavy chain residues Leu204 to His365 and Pro371 to Leu438 (numbering according to the sequence of rat propPPI). Residues Ala119, Thr366 to Ser369 and Asp370 are not well defined by the electron density and the residues that constitute the activation peptide (approximately Asn120 to Gln202, Ile203, Leu204 or Ser205) are not found in the mature enzyme. In accord with previous finding, a few activation peptide residues (at least Leu204 and Ser205) are attached to the N-terminus of the heavy chain (Lauritzen et al. (1998) Protein Expr. Purif. 14, 434-442). Recombinant rat DPPI was characterised as a dimer in solution (Lauritzen et al. (1998) Protein Expr. Purif. 14, 434-442) but crystallised as a tetramer in accordance with the oligomeric structure of the enzyme in vivo. The space group is P6₄₂₂ and the unit cell dimensions are a=166.24 Å, b=166.24 Å, c=80.48 Å with α=β=90° and γ=120°.

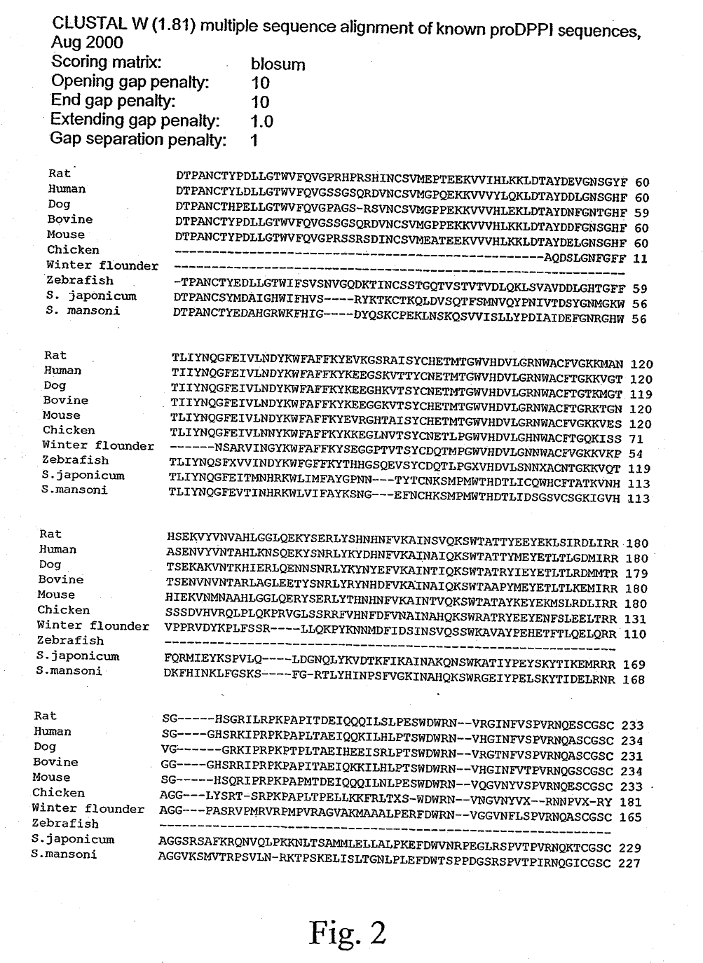

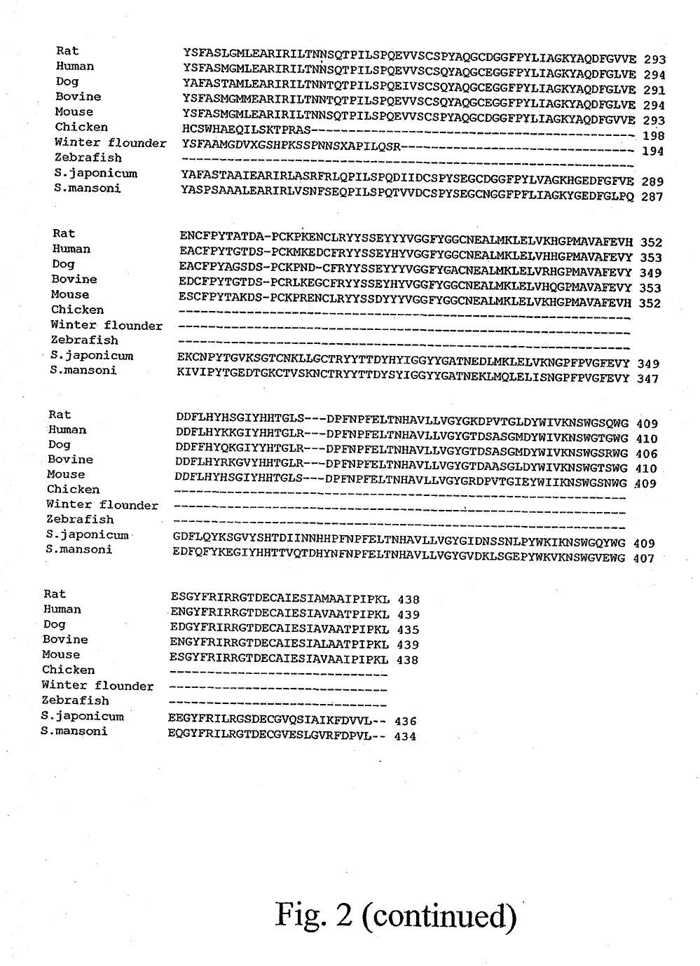

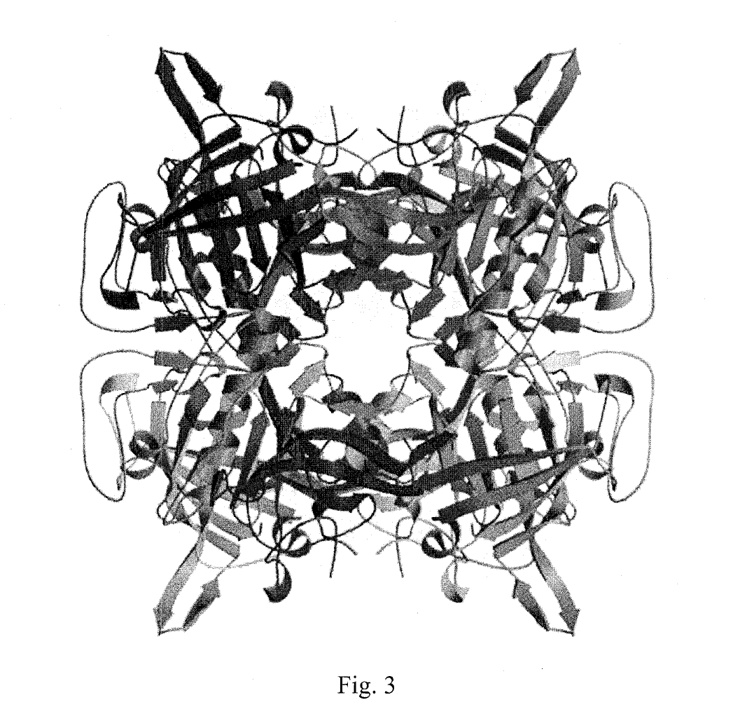

All related peptidases are monomers and the disclosed structure reveals for the first time the types of interfaces that are found between the four subunits. The crystal structure of the present invention shows that the subunits are assembled in a ring-like structure with the residual pro-parts and catalytic domains of neighbouring subunits being assembled head-to-tail so that each kind of domain points upwards and downwards, alternately, and the active sites point away from the centre of the ring (FIG. 3). By this arrangement, the group of residues that form contacts at an interface between two subunits is the same in both subunits. At one rat DPPI subunit interface, residues V54, D74, D104, Y105, L106, R108, L249, Q287, L313, Y316, S318, I435, P436 and K437 (underlined residues are identical in rat and human DPPI according to the sequence alignment in FIG. 2) are about 5 Å or closer to one or more residues of the same group in the neighbouring subunit. At a different kind of rat DPPI subunit interface, residues K45, K46, T49, Y51, C330, N331, E332, F372 and G419 (underlined residues are identical in rat and human DPPI according to the sequence alignment in FIG. 2) are about 5 Å or closer to one or more residues of the same group in the neighbouring subunit. Other residues may also contribute to subunit interface formation. While every subunit is in close contact with its two neighbouring subunits, no interaction with the third subunit is observed across the ring-like tetrameric structure.

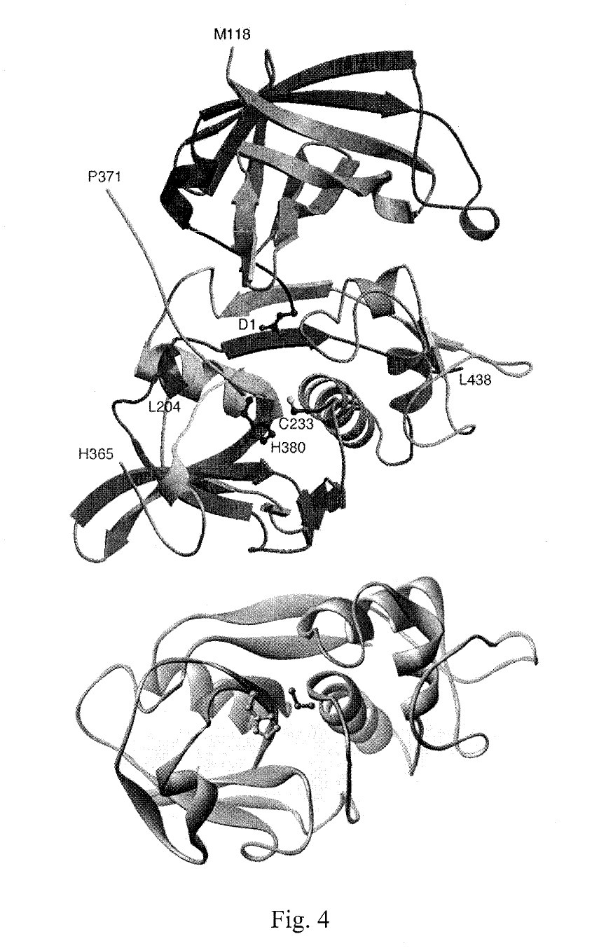

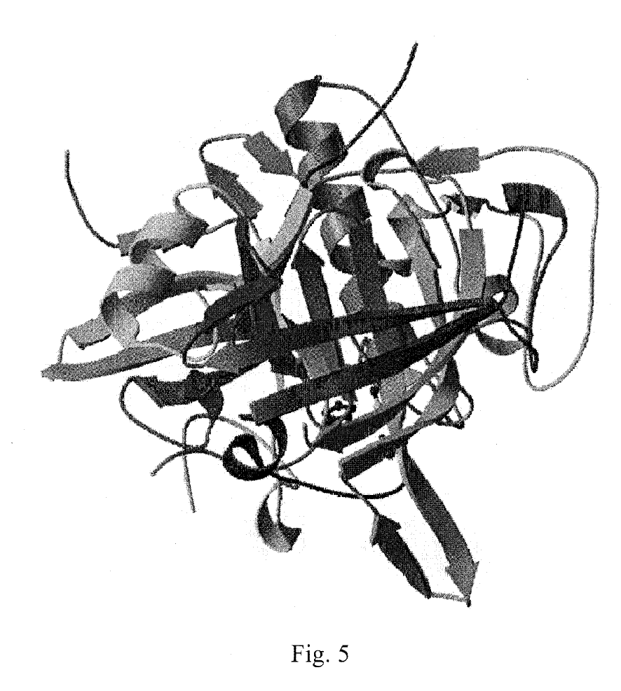

As expected on basis of sequence similarity to the catalytic domains of papain family peptidases, the present invention shows that the catalytic domain of rat DPPI has a similar fold (FIGS. 4 and 5). The fold of the residual pro-part, its interaction with the catalytic domain and role in tetramer formation, however, has previously not been known. The crystal structure of the present invention thus reveals that residues 1-119 form a well-defined beta-barrel domain with little or no alpha helical structure. Interestingly, residues Lys82-C94 form a beta-hairpin that projects away from the barrel and into solution. This unusual feature may be a crystal packing artefact, though, because these loops interact with residues in other tetramers. The residual pro-part domain is shown to be bound to the catalytic domain through contacts to both the heavy and light chains. Residual pro-part residues, including D1, I28, T61, L62, I63, Y64, E69, K76, F78, W101 and H103, are located about 5 Å or closer to one or more of the heavy chain residues P268, Y269, Q271, Y279, L280, K284, D288, G324, G325 and F326 (underlined residues are identical in rat and human DPPI according to the sequence alignment in FIG. 2). Similarly, residual pro-part residues, including T7, Y8, P9, Y64 and N65, are located about 5 Å or closer to one or more of the light chain residues F372, N373, L377 and T378 (underlined residues are identical in rat and human DPPI according to the sequence alignment in FIG. 2).

In the present invention, the residual pro-part domain is shown to be located relative to the catalytic domain in a way so that it blocks the extreme end of the unprimed active site cleft. Most significantly, the N-terminus of the residual pro-part projects further towards the catalytic residues and the free amino group of the conserved Asp1 is held in position by a hydrogen bond to the backbone oxygen atom of Asp274. This arrangement is most certainly very important in providing a negative charge, located on the side chain of Asp1, in a fixed position within the active site cleft. The delocalised negative charge that this residue carries under physiological conditions on its OD1 and OD2 oxygen atoms is localised about 7.4 and 8.7 Å from the sulphur atom of the catalytic Cys233 residue. This distance together with the dipeptidyl aminopeptidase specificity of rat DPPI strongly indicates that the protonated N-termini of peptide substrates form a salt bridge to the negative charge on the side chain of Asp1. Furthermore, the position of the N-terminal Asp1 residue is fixed by a hydrogen bond between the free amino group of this residue (hydrogen bond donor) and the backbone carbonyl oxygen of Asp274 (hydrogen bond acceptor). The donation of a negative charge in the active site cleft of a cysteine peptidase by the side chain of the N-terminal residue of the residual pro-part is a novel structural feature not previously observed. Thus the present invention provides a novel and surprising principle for substrate binding which is very different from the binding of the substrate N-terminus by the negative charge on the C-terminal of the cathepsin H “mini-chain” (Guncar, G. et al. (1998) Structure 6, 51-61). Therefore, in one embodiment of the present invention a model is proposed that can be used to elucidate the substrate binding of other DPPI-like enzymes and which might even be employable for other peptidases not belonging to the family of cathepsin peptidases. Another embodiment of the present invention relates to the use of said information for testing and/or rationally or semi-rationally designing a chemical compound which binds covalently or non-covalently to a protein with at least 37% amino acid sequence identity to the amino acid sequence of rat DPPI protein as shown in SEQ ID NO:1, characterised by applying in a computational analysis structure co-ordinates of a crystal structure as described above and in Table 2.

Between Asp1 and Cys233, a wide and deep pocket is found, which may accommodate the side chains of one or both of the two most N-terminal substrate residues. In addition to Asp1 and Cys233, this pocket is defined by residual pro-part, heavy chain and light chain residues including, but not limited to, Tyr64, Gly231, Ser232, Tyr234, Ala237, Asp274, Gly275, Gly276, Phe277, Pro278, Thr378, Asn379, His380, Ala381. These residues are identical in rat and human DPPI according to the sequence alignment in FIG. 2 except for Asp274, which is a glutamic acid in human DPPI. Both aspartic acid and glutamic acid residues are acidic residues. Accordingly, the active sites in rat and human DPPI can be expected to be structurally very similar and a very good and usable model of the active site of human DPPI and possibly of most of mammalian DPPI can be built using structure co-ordinates of rat DPPI and visa versa. Furthermore, very good models of other closely related DPPI enzymes, such as but not limited to the other mammalian DPPIs included in FIG. 2, can possibly be built using the structural co-ordinates of rat or human DPPI or both.

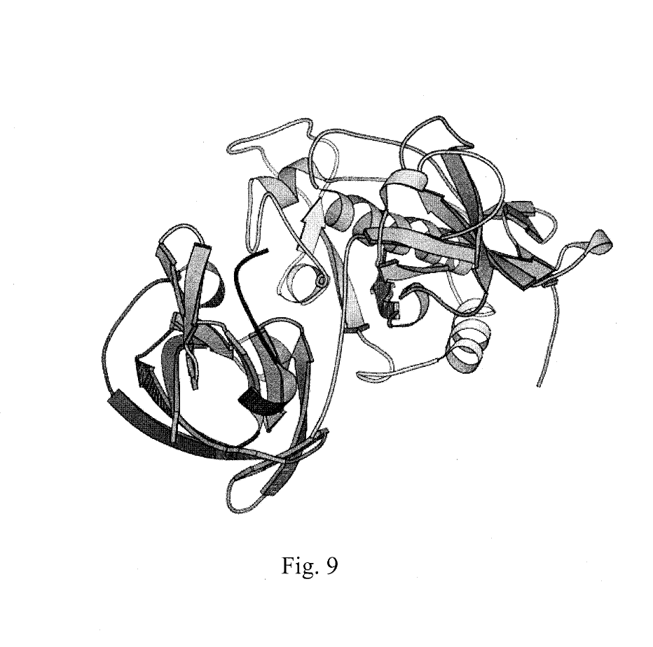

An illustrative example is a human DPPI model based on the structural data of rat DPPI. FIG. 9 shows a model of the structure of human DPPI made based on the structural data of rat DPPI. FIGS. 10-15 shows the human structure based on the structural co-ordinates of human DPPI as provided in table 2b. It is clear for the skilled person that these two structures resembles each other and the model, based on the rat data, is a good model.

A crystal structure and/or the structural co-ordinates of human DPPI are preferred embodiments of the present invention.

Native as well as recombinant rat DPPI is known to be glycosylated. The innermost sugar rings of the carbohydrate chains attached to Asn5 and Asn251 are defined by the electron density.

Production of DPPI for Crystallisation

The present invention provides, for the first time, a crystal of rat DPPI as well as the structure of the enzyme as determined therefrom. Further, for the first time is also disclosed the structural co-ordinates for human DPPI. Therefore, when herein is discussed the use of rat DPPI co-ordinates it should be understood that the same use of the human co-ordinates are also within the scope of the invention. Accordingly, one aspect of the invention resides in the obtaining of enough DPPI protein of sufficient quality to obtain crystals of sufficient quality to determine the three dimensional structure of the protein by X-ray diffraction methods. One embodiment of the present invention thus relates to obtaining a crystallisable composition comprising a substantially pure protein described by an amino acid sequence which is at least 37%, such as at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of rat DPPI protein as shown in SEQ ID NO:1 and to the composition itself.

The present invention further relates to an already crystallised molecule or molecular complex comprising a rat DPPI protein with the amino acid sequence as shown in SEQ. ID. NO. 1 SEQ ID NO:1 and/or a protein with at least 37% such as at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity to the amino acid sequence of rat DPPI protein as shown in SEQ ID NO:1.

Human and rat DPPI had previously been purified from natural sources like kidney, liver or spleen, e.g. as described by (Doling et al. (1996) FEBS Lett. 392, 277-280), but often in low amounts and often as preparations characterised by inhomogeneous, partially degraded (Cigic et al. (1998) Biochim. Biophys. Acta 1382, 143-150) and impure protein limiting the possibility of growing crystals of sufficient quality.

The baculovirus/insect cell expression system used to obtain the crystallisable composition of the present invention, which was recently developed for the production of DPPI from a recombinant source (Lauritzen et al. (1998) Protein Expr. Purif. 14, 434-442), offers the advantages of having strong or moderately strong promoters available for the high level expression of a heterologous protein. The baculovirus/insect cell system is also able to resemble eukaryotic processing like glycosylation and proteolytic maturation.

Furthermore, the recombinant human and rat DPPIs obtained with the baculovirus/insect cell system are very similar to their natural counterparts with respect to glycosylation, enzymatic processing, oligomeric structure, CD spectroscopy and catalytic activity. In one embodiment of the present invention, recombinant protein was used that was produced in this expression system rendering it possible to obtain crystals of sufficient quality to determine the three-dimensional structure of mature rat DPPI to high resolution.

Considering the high homology of the proteins in the DPPI family, one aspect of the invention relates to the use of the structure co-ordinates of the recombinant rat DPPI crystals to solve the structure of crystallised homologue proteins, such as but not limited to dog, murine, monkey, rabbit, bovine, porcine, goat, horse, chicken or turkey DPPI. Homologues may be isolated from natural sources such as spleen, kidney, liver, lung or placenta by use of one or more of a variety of conventional chromatographic and fractionation principles such as hydrophobic interaction chromatography, anion-exchange chromatography, cation exchange chromatography, high performance liquid chromatography (HPLC), affinity chromatography or precipitation, or the homologues proteins may be produced as recombinant proteins.

Lengthy table referenced here
US20110236367A1-20110929-T00001
Please refer to the end of the specification for access instructions.

Lengthy table referenced here
US20110236367A1-20110929-T00002
Please refer to the end of the specification for access instructions.

Another aspect of the invention is the use of the structure co-ordinates of mature rat DPPI to solve the structure of crystals of co-complexes of wild type or mutant or modified forms of DPPI. DPPI can furthermore be isolated from a recombinant source. Crystals of co-complexes may be formed by crystallisation of e.g. DPPI from a natural or a recombinant source covalently or non-covalently associated with a chemical entity or compound, e.g. co-complexes with known DPPI inhibitors such as E-64 or Gly-Phe-CHN₂. The crystal structures of such complexes may then be solved by molecular replacement, using some or all of the atomic co-ordinates disclosed in this invention, and compared with that of wild-type DPPI. Detailed analysis of the location and conformation of such known DPPI inhibitors, of their interactions with DPPI active site cleft residues and of the structural arrangement of said active site cleft residues upon binding of inhibitors will provide information important for rational or semi-rational design of improved inhibitors. Furthermore, structural analysis of DPPI-inhibitor co-complexes may reveal potential sites for modification within the active site of the enzyme, which can be changed to increase or decrease the enzyme's sensitivity to one or more protease inhibitors, preferably without affecting or reducing the catalytic activity of the enzyme.

The present invention furthermore relates to the use of the structural information for the design and production of mutants of DPPI, fusion proteins with DPPI, tagged forms of DPPI and new enzymes containing elements of DPPI, and the solving of their crystal structure. More particularly, by virtue of the present invention, e.g. the knowledge of the location of the active site, chlorine binding site and interface between the different domains/subunits constituting DPPI permits the identification of desirable sites for mutation and identification of elements usable in design of new enzymes. For example, mutation may be directed to a particular site or combination of sites of wild-type DPPI, i.e., the active site, the chlorine binding site, the glycosylation sites or a location on the interface sites between the domains/subunits may be chosen for mutagenesis. Similarly, a location on, at, or near the enzyme surface may be replaced, resulting in an altered surface charge, as compared to the wild-type enzyme. Alternatively, an amino acid residue in DPPI may be chosen for replacement based on its hydrophilic or hydrophobic characteristics.

The mutants or modified forms of DPPI prepared by this invention may be prepared in a number of ways. For example, the wild-type sequence of DPPI may be mutated in those sites identified using the present invention as desirable for mutation, by means of site directed mutagenesis by PCR or oligonucleotide-directed mutagenesis or other conventional methods well known to the person skilled in the art. Synthetic oligonucleotides and PCR methods known in the art can be used to produce translational fusions between the 5′ or 3′ end of the entire DPPI coding sequence or fragments hereof and fusion partners like sequences encoding proteins or tags, e.g. polyhistidine tags. Alternatively, modified forms of DPPI may be generated by replacement of particular amino acid(s) with unnaturally occurring amino acid(s) e.g. selenocysteine or selenomethionine or isotopically labelled amino acids. This may be achieved by growing a host organism capable of expressing either the wild type or mutant polypeptide on a growth medium depleted of the natural amino acids but enriched in the unnatural amino acids.

According to this invention, a mutated/altered DPPI DNA sequence produced by the methods described above, or any alternative methods known in the art, and also the above mentioned homologues DPPIs, originating from species other than human and rat, can be recombinantly expressed by molecular cloning into an expression vector and introducing the vector into a host organism.

In an especially preferred embodiment of the invention, a host-vector system like the one used for production of protein for crystallisation is employed wherein the host is an insect cell such as cells derived from Trichoplusia ni or Spodoptera frugiperda and the vector is a baculovirus vector such as vectors of the type of Autographica californica multiple nuclear polyhedrosis virus or Bombyx mori nuclear polyhedrosis virus. However, any of a wide variety of well-known available expression vectors and hosts is useful to express the mutated/modified/homologues DPPI coding sequences of this invention.

An expression vector, as is well known in the art, typically contains a suitable promoter and other appropriate regulatory elements required for transcription of cloned copies of genes and the translation of their mRNAs in an appropriate host. A vector may also contain elements that permit autonomous replication in a host cell independent of the host genome, and one or more phenotypic markers for selection purposes. In some embodiments, where secretion of the produced protein is desired, nucleotides encoding a “signal sequence” may be inserted in front of the mutated/modified/homologues DPPI coding sequence. For expression under the direction of the control sequences, a desired DNA sequence must be operatively linked to the control sequences, i.e., they must have an appropriate start signal in front of the DNA sequence encoding the DPPI mutant, modified form of DPPI or homologues DPPI and maintain the correct reading frame to permit expression of that sequence under the control of the control sequences and production of the desired product encoded by that DPPI sequence.

Such vectors include but are not limited to, bacterial plasmids, e.g., plasmids from E. coli including coli E1, pCR1, pBR322, pMB9 and their derivatives, wider host range plasmids, e.g., RP4, phage DNAs, e.g., the numerous derivatives of phage lambda, e.g., NM 989, and other DNA phages, e.g., M13 and filamentous single stranded DNA phages, yeast plasmids, vectors derived from combinations of plasmids and phage DNAs, such as plasmids which have been modified to employ phage DNA or other expression control sequences, cosmid DNA, virus, e.g., vaccinia virus, adenovirus or baculovirus.

The vector must be introduced into host cells via any one of a number of techniques comprising transformation, transfection, infection, or protoplast fusion. A wide variety of hosts are useful for producing mutated/modified/homologues DPPI according to this invention. These hosts include, for example, bacteria, such as E. coli, Bacillus and Streptomyces species, fungi, such as yeasts, e.g. Saccharomyces cerevisiae, Pichia astoris, Hansenula polymorpha, animal cells, such as CHO and COS-1 cells, insect cells, such as Drosophila cells, Trichoplusia ni or Spodoptera frugiperda, plant cells, transgenic host cells and whole organism such as insects.

In selecting a host-vector system, a variety of factors should also be considered. These include, for example, the relative strength of the system, its controllability, and its compatibility with the DNA sequence encoding the modified DPPI of this invention. Hosts should be selected by consideration of their compatibility with the chosen vector, the toxicity of the mutated/modified/homologues DPPI to them, their ability to secrete proforms or mature products, their ability to fold proteins correctly, Their ability of proteolytical processing and oligomerization, their fermentation requirements, the ease of the purification of the DPPI protein from them and safety. Within these parameters, one of skill in the art may select various vector/expression control system/host combinations that will produce useful amounts of the DPPI protein.

The mutants, modified forms of DPPI or homologues DPPI produced in these systems may be purified by a variety of conventional steps and strategies. In the present invention, extracellular partially matured rat DPPI is isolated by ammonium sulphate fractionation, hydrophobic interaction chromatography, desalting and anion-exchange chromatography. Other chromatographic and fractionation principles may also be used in purification of modified forms of DPPI, e.g. purification by cation exchange chromatography, high performance liquid chromatography (HPLC), immobilised metal affinity chromatography (IMAC), affinity chromatography or precipitation.

Once the mutant or modified DPPI has been generated, the protein may be tested for any one of several properties of interest. For example, mutated or modified forms may be tested for DPPI activity by spectrophotometric measurement of the initial rate of hydrolysis of the chromogenic substrate Gly-Phe-p-nitroanilide (Lauritzen et al. (1998) Protein Expr. Purif. 14, 434-44). Mutated and modified forms may be screened for higher or lower specific activity in relation to the wild-type DPPI. Furthermore, mutants or modified forms may be tested for altered DPPI substrate specificity by measuring the hydrolysis of different peptide or protein substrates.

Mutants or modified forms of DPPI may be screened for an altered charge at physiological pH. This is determined by measuring the mutant DPPI isoelectric point (pl) in comparison with that of the wild type parent. The Isoelectric point may be measured by gel-electrophoresis. Further properties of interest also include mutants with increased stability to subunit dissociation.

Mutants or modified forms of DPPI or new homologues may alternatively also be crystallised to again yield new structural data and insights into the protein structure of dipeptidyl peptidases and/or related enzymes. Thus, one embodiment of the present invention relates to a crystallised molecule or molecular complex of a DPPI or DPPI-like protein, in which said molecule is mutated prior to being crystallised.

Chemical Modification of DPPI

The present invention further holds chemical modification of DPPI and/or a variant hereof which may be performed to characterise the protein or to obtain a protein with altered properties. In both cases, X-ray crystallographic analysis of the modified protein may provide valuable information about the site(s) of modification and structural arrangement of the organic or inorganic chemical compound and of the DPPI residues that interact with said compound. One aspect of the present invention therefore relates to a crystallised molecule or molecular complex, in which said molecule is chemically and/or enzymatically modified. Another aspect of the present invention subsequently relates to the crystal structure of a so modified protein itself.

Characterisation of DPPI or DPPI-like proteins by modification with organic or inorganic chemical compounds and, optionally, X-ray crystallography could be performed by reacting said DPPI or DPPI-like protein with e.g. inhibitory compounds, fluorescent labels, iodination reagents or activated polyethylen glycol (“PEGylation”) or other polyhydroxy polymers. The inhibitory compounds could be compounds that bind covalently to the active site cysteine residues or at accessory binding sites. X-ray crystallographic analysis of such modified DPPI or DPPI-like protein would give information important for the further development of more potent and more specific inhibitors. Fluorescent labelling and iodination of DPPI or DPPI-like proteins would permit tracing the molecules and give information about the molecular environment of fluorescent group(s). Compounds such as fluorescein-5-maleimide and fluorescein isothiocyanate, which react specifically with cysteine residues and primary amines, respectively, can be utilised to attach fluorescent labels to certain kinds of functional groups within proteins and K^{125I}, K^{131I}, Na^{125I} or Na^{131I} can be used for iodination of tyrosine residues. Determination by X-ray crystallography of the sites of tyrosine iodination and of attachment of fluorescent groups in particular may be essential for interpreting results from protein-protein interaction studies (binding of receptors, inhibitors, cofactors etC) and in analyses of structural rearrangements.

PEGylation is another common method of chemically modifying proteins whose crystal structure is enscoped by the present invention granted that their amino acid sequence is at least 37% identical with the amino acid of rat DPPI as shown in FIG. 1. In the pharmaceutical industry, PEGylation is used to increase circulating half-life and resistance to proteolysis, decrease immunogenecity and enhance solubility and stability of protein drugs.

Uses of the Structure Co-Ordinates of DPPI

For the first time, the present invention permits a detailed atomic and functional description of DPPI, including descriptions of the structure of the active site, of the chlorine ion binding site, of the residual pro-part and of the interfaces between the subunits and between the catalytic and residual pro-part domains. The present invention thus enables the design, selection and synthesis of chemical compounds, including inhibitory compounds, capable of binding to DPPI, including binding at the active sites of DPPI or at intramolecular interfaces. The invention can also be used to identify and characterise accessory binding sites. Furthermore, this invention can be used to rationally and semi-rationally design mutants of DPPI with altered or improved characteristics and to theoretically model and facilitate experimental determination by X-ray crystallography the structures of homologous proteins, including related DPPIs from other species.

Therefore, the present invention provides a method for selecting, testing and/or rationally or semi-rationally designing a chemical compound which binds covalently or non-covalently to a protein with at least 37% amino acid sequence identity to the amino acid sequence of rat DPPI protein as shown in SEQ ID NO:1, characterised by applying in a computational analysis structure co-ordinates of a crystal structure according to table 2. In a preferred embodiment, the method for identifying a potential inhibitor of an enzyme with at least 37% amino acid sequence identity to the amino acid sequence of rat DPPI protein as shown in SEQ ID NO:1, provided comprises using the atomic co-ordinates of a crystallised molecule or molecular complex according to table 2 to define the catalytic active sites and/or an accessory binding site of said enzyme, identifying a compound that fits the active site and/or an accessory binding site so identified, obtaining the compound, and contacting the compound with a DPPI or DPPI-like protein to determine the binding properties and/or effects of said compound on and/or the inhibition of the enzymatic activity of DPPI by said compound. This method can be performed on the atomic co-ordinates of a crystallised molecule or molecular complex having an at least 37% identical amino acid sequence with rat DPPI and which are obtained by X-ray diffraction studies.

Potential Effects of DPPI Binding Compounds

Compounds that bind to DPPI many alter the properties of the enzyme or its proenzyme.

For instance, a chemical compound that binds at or close to the active site or causes a structural rearrangement of DPPI upon binding may inhibit or in other ways modify the catalytic activity of the active enzyme and a compound that binds at a subunit or domain interface may cause stabilisation or destabilisation of the native, oligomeric structure. Furthermore, DPPI binding compounds may decrease or increase the in vivo clearance rate, solubility and catalytic activity of the enzyme or alter the enzymatic specificity.

Identification of Ligand Binding Sites

Knowledge of the atomic structure of DPPI enables the identification and detailed atomic analyses of ligand binding sites essential for rational or semi-rational design of DPPI binding compounds, including DPPI inhibitors. Such ligands may interact with DPPI through both covalent and non-covalent interactions and must be able to assume conformations that are structurally compatible with the DPPI ligand binding sites. The locations of the active sites of DPPI subunits can be determined by the localisation of the catalytic cysteine and histidine residues (Cys234 and His381 in human DPPI, respectively; see FIG. 2). Accessory binding sites may be identified by persons skilled in the art by visual inspection of the molecular structure and by means of computational methods, e.g. by using the MCSS program (available from Molecular Simulations, San Diego, Calif.).

Design and Screen of Inhibitors

Once a DPPI or propPPI ligand binding site has been selected for targeting, computer based modelling, docking, energy minimisation and molecular dynamics techniques etC may be used by persons skilled in the art to design ligands or ligand fragments that bind to DPPI, to evaluate the quality of fit and strength of interaction and to further develop and optimise selected compounds. In another aspect of the invention, compounds may be screened by computational means for their ability to bind to the surface of DPPI without defining a specific site of interaction. In yet another aspect of the invention, random or semi-random ligand libraries may be screened prior to its actual synthesis. In general, computational methods can be used for selecting and optimising DPPI binding ligands, but the actual biochemical and pharmacological properties of any given ligand must be determined experimentally.

The knowledge about the crystal structure of DPPI and/or DPPI-like proteins, provided in the present invention, allows for identifying a potential inhibitor of a DPPI or DPPI-like protein whereby all or some of the atomic co-ordinates of a crystal structure of a DPPI or DPPI-like protein is used to define the catalytic active sites or accessory binding sites of an enzyme with at least 37% amino acid sequence identity to the amino acid sequence of rat DPPI protein as shown in SEQ ID NO:1, a compound is identified that fits such an active site or accessory binding site, a compound is obtained, and said compound is contacted with a DPPI or DPPI-like protein in the presence of a substrate in solution to determine the inhibition of the enzymatic activity by said compound.

In another embodiment of the present invention, a method is provided for designing a potential inhibitor of a DPPI or DPPI-like protein comprising providing a three dimensional model of the receptor site in an enzyme with at least 37% amino acid sequence identity to the amino acid sequence of rat DPPI protein as shown in SEQ. ID. NO. 1, and a known inhibitor, locating the conserved residues in the known inhibitor which constitute the inhibition binding pocket, and designing a new a DPPI or DPPI-like protein inhibitor which possesses complementary structural features and binding forces to the residues in the known inhibitor's inhibition binding pocket.

Said identified compound and/or potential inhibitor can either be designed de novo or be designed from a known inhibitor or from a fragment capable of associating with a DPPI or DPPI-like protein. Said known inhibitor is preferably selected from the group consisting of dipeptide halomethyl ketone inhibitors, dipeptide diazomethyl ketone inhibitors, dipeptide dimethylsulphonium salt inhibitors, dipeptide nitril inhibitors, dipeptide alpha-keto carboxylic acid inhibitors, dipeptide alpha-keto ester inhibitors, dipeptide alpha-keto amide inhibitors, dipeptide alpha-diketone inhibitors, dipeptide acyloxymethyl ketone inhibitors, dipeptide aldehyde inhibitors and dipeptide epoxysuccinyl inhibitors. And is often constructed of chemical entities or fragments capable of associating with a protein with at least 37% amino acid sequence identity to the amino acid sequence of rat DPPI protein as shown in SEQ ID NO:1, and reassembled after the testing procedure into a single molecule to provide the structure of said potential inhibitor.

Specialised computer programs are available to persons skilled in the art of structure based drug design to computationally design, evaluate and optimise DPPI ligands. DPPI binding ligands are generally designed either by connecting small ligand site binding molecules (identified using e.g. MCSS which is available from Molecular Simulations, San Diego, Calif.) using computer programs such as Hook (Molecular Simulations, San Diego, Calif.) or by “de novo” design of whole ligands using computer programs such as Ludi (available from Molecular Simulations, San Diego, Calif.) and LEAPFROG® (available from Tripos, St. Louis, Mo.).

To evaluate the quality of fit and strength of interactions between ligands or potential ligands and DPPI ligand binding sites, docking programs such as Autodock (available from Oxford Molecular, Oxford, UK), Dock (available from Molecular Design Institute, University of California San Francisco, Calif.), Gold (available from Cambridge Crystallographic Data Centre, Cambridge, UK) and FlexX and FlexiDock (both available from Tripos, St. Louis, Mo.) may be used. These programs and the program Affinity (available from Molecular Simulations, San Diego, Calif.) may also be used in further development and optimisation of ligands. Standard molecular mechanics forcefields such as CHARMm® and AMBER may be used in energy minimisation and molecular dynamics.

The present invention thus provides the means to test and/or identify new or improved binding substances to DPPI and therefore a so identified and obtained chemical compound and/or potential inhibitor is of course enscoped in the present invention.

Determination of Structures of Homologous Proteins

By using the structural co-ordinates (in whole or in part) disclosed in the present invention in molecular replacement, it is generally possible for a person skilled in the art to rapidly determine the phases of diffraction data obtained from X-ray crystallographic analysis of crystals of homologous DPPIs, including dog, mouse, bovine and blood fluke DPPI, of DPPI mutants, of DPPIs in complexes with ligands and of any combination hereof.

Any phase information in the diffracted X-rays is lost upon data collection and has to be restored in order to determine the position and orientation of the molecule within the crystal, calculate the first density map and initiate model building. Without a homologous structure, which can be used as a search model, the phases have to be determined experimentally from comparison of diffraction data obtained with crystals of the native enzyme and of heavy atom derivatives of the enzyme. This method of phase determination can be slow and laborious, as good heavy atom derivative data sets can be very difficult to obtain. In contrast, phase determination by molecular replacement is generally fast if an appropriate search model is available.

Phase determination by molecular replacement generally involves the following steps:

1) Determination of the position and orientation of the crystallised molecule within the crystal using rat or human DPPI as search model. Specialised computer programs such as AMoRe (Navaza (1994) Acta Cryst. A50, 157-163) or XSight® (available from Molecular Simulations, San Diego, Calif.) are available for this task.

2) Having successfully determined a set of initial phases, the first density map, which shows the approximate locations of fixed atoms, can be calculated using computer programs such as MAIN (D. Turk: Proceedings from the 1996 meeting of the International Union of Crystallography Macromolecular Macromolecular Computing School, eds P. E. Bourne & K. Watenpaugh).

3) A model of the crystallised protein is build into the calculated density map.

4) The structure is refined during one or more cycles of automated refinement using programs such as X-PLOR® (available from Molecular Simulations, San Diego, Calif.) and manual rebuilding. Optionally, the electron density map may be improved by solvent flattening and noncrystallographic symmetry averaging.

Modelling of the Structures of Homologous Proteins

In another aspect of the invention, the determined structure co-ordinates, or partial structure co-ordinates, of rat DPPI can be used, directly or indirectly, by persons skilled in the art, to model the structures of homologous proteins, for example DPPIs from other species, including dog, mouse, bovine and blood fluke DPPI, and mutant forms of DPPI. Knowledge of the structure of rat DPPI represents a unique and essential basis for modelling of other DPPI structures.

Firstly, the residual pro-port, which is retained in the mature form of DPPI and which is now known to be indispensable for maintaining the oligomeric structure of the enzyme, shares no detectable sequence homology to any other amino acid sequence, including the amino acid sequences of the known Cl family peptidase, or to translated nucleotide sequence in the publicly available databases (Swiss-Prot™, GenBank® etC). Accordingly, no currently known technique or method is available for modelling the residual pro-part of DPPI without the information about the residual rat pro-part structures which is disclosed in this invention.

Secondly, modelling DPPI structures on basis of the already known and publicly available X-ray structures of e.g. cathepsins H, L, S, B and K has problems because the catalytic domain of DPPI is formed by two peptide chains, the heavy chain carrying the catalytic cysteine residue and the light chain carrying the catalytic histidine residue. Chain cleavages within this domain are also observed in the homologous proteases but the site of cleavage in DPPI is unique to this enzyme and, importantly, no currently published homologous X-ray structure has a chain cleavage in this position. Because of this, the modeller faces an apparent lack of modelling template. The importance of this is demonstrated in the structures of rat and human DPPI in which significant spatial separations of the newly formed peptide chain termini following cleavage are revealed. Furthermore, because the cleavage site between the heavy chain and the light chain (cleavage between pro-DPPI residues R370 and D371) is close (10 residues) to the catalytic histidine residue, the impacts of the chain cleavage on the topology of the active site and the active site residues would be impossible to predict accurately.

Preferably, models of DPPIs, for which the structures are not known, are build by homology modelling and generally comprises the steps of:

1) Aligning the amino acid sequence of the protein to be modelled with the sequence of rat DPPI or human DPPI. Alternatively, all three sequences may be aligned. A preferred program for aligning two or more homologous amino acid sequences is Clustal W 1.8 (Thompson et al. (1994) Nucleic Acids Res. 22, 4673-4680);

2) An initial model is built on a suitable computer with molecular modelling software by incorporating the protein sequence into the structure of rat or human DPPI in accordance with the alignment. Alternatively, if all three protein sequences were aligned in step 1, the rat DPPI structure is first superimposed and the model structure is subsequently build on basis of both structures;

3) The modelled structure may then be subjected to energy minimisation using standard force fields such as CHARMm® or AMBER;

4) The energy-minimised model is remodelled in regions where stereochemistry restraints are violated and to correct bad contacts, bond distances, bond angles and torsion. Information from side chain rotamer and structure libraries may be used in modelling of low homology and/or flexible regions such as loop regions;

5) Optionally, molecular dynamics and more rounds of energy minimisation may be performed. Specialised computer programs such as Modeler and Homology (available from Molecular Simulations, San Diego, Calif.) and are used by persons skilled in the art to perform automatic or semi-automatic homology model construction. A review on homology modelling can be found in Rodriguez et al. (1998).

Therefore, a method is provided in the present invention for selecting, testing and/or rationally or semi-rationally designing a modified protein with at least 37% amino acid sequence identity to the amino acid sequence of rat DPPI protein as shown in SEQ ID NO:1, characterised by applying any of the atomic co-ordinates as shown in table 2, and/or the atomic co-ordinates of a crystal structure modelled after said co-ordinates.

The present invention furthermore relates to the use of any of the atomic co-ordinates according shown in table2 and/or the atomic co-ordinates of a crystal structure modelled after said co-ordinates for the identification of a potential inhibitor of a DPPI or DPPI-like protein and/or for the modification of a protein with at least 37% amino acid sequence identity to the amino acid sequence of rat DPPI protein as shown in SEQ ID NO:1, such that it can catalyse the cleavage of a natural, unnatural or synthetic substrate more efficiently than the wild type enzyme.

Such substrates are typically selected from the group consisting of dipeptide amides and esters; dipeptides C-terminally linked to a chromogenic or fluorogenic group, polyhistidine purification tags and granule serine proteases with a natural dipeptide propeptide extension.

Following homology modelling, the quality of the model structure can be estimated using specialised computer programs such as PROCHECK (Laskowski et al. (1993) J. Appl. Cryst. 26, 283-291) and Verify3D (Luthy et al. (1992) Nature 356, 83-85).

Rational and Semi-Rational Design of DPPI Mutants

The present invention further provides a method for theoretically modelling the structure of a first protein with at least 37% amino acid sequence identity to the amino acid sequence of rat DPPI protein as shown in SEQ ID NO:1, characterised by

a) Aligning the sequence of said first protein with the sequence of a second protein with known crystal structure or structural co-ordinates according to any of claims 16-28, and incorporating the first sequence into the structure of the second polypeptide, thereby creating a preliminary structural model of said first protein,

b) Subjecting said preliminary structural model to energy minimisation, resulting in an energy minimised model,

c) Remodelling the regions of said energy minimised model where stereochemistry restraints are violated, and

d) Obtaining structure co-ordinates of the final model.

On basis of the detailed atomic and functional description of DPPI enabled by this invention, a rational or semi-rational selection of desirable amino acid residues for mutation is enabled. Such mutants can be used to further characterise the role and importance of specific residues and regions within e.g. the active site, the chlorine ion binding site, the residual pro-part and the interfaces between the subunits and between the catalytic and residual pro-part domains. Also, knowledge of the structure co-ordinates of DPPI aid in selecting amino acid residues for mutagenesis with the purpose of altering the properties of DPPI. For example, it could be desirable to increase e.g. the thermostability, the stability towards chaotropic agents and detergents, the stability at alkaline pH, or the catalytic efficiency (k_{cat}/K_{M}) or to alter the catalytic specificity. Also, it could be desirable to alter the oligomeric structure of DPPI, to enhance the intramolecular interactions between the DPPI subunits or domains or to produce mutants of DPPI with reduced sensitivity to inhibitors of the cystatin family of cysteine peptidase inhibitors, in particular human cystatin C Furthermore it could be desirable to design mutants of DPPI with different ratios between aminopeptidase and transferase activity and reduced levels of substrate restrictions making them suitable for effective enzymatic synthesis or semisynthesis of peptides and proteins

A number of methods are available for a person skilled in the art for preparing random or directed mutants of DPPI. For example, mutations can be introduced by use of oligonucleotide-directed mutagenesis, by error-prone PCR, by UV-light radiation, by chemical agents or by substituting some of the coding region with a different nucleotide sequence either produced by chemical synthesis or of biological origin, e.g. a nucleotide sequence encoding a fragment of DPPI from different species.

Random and directed mutants of DPPI can typically be expressed and purified by the same methods as described for expression and purification of wild type DPPI.

Once the mutant forms of DPPI are obtained, the mutants can be characterised or screened for one or more properties of interest. For example, the catalytic aminopeptidase efficiency can be evaluated using Gly-Phe-p-nitroanilide, Ala-Ala-p-nitroanilide, or Gly-Arg-p-nitroanilide as substrate. Alternatively, the chromogenic leaving group p-nitroanilide can be replaced with a fluorescent-leaving group, e.g. 4-methoxy naphtylamide. Mutants with altered substrate specificity, e.g. mutants which can cleave peptides with N-terminal basic residues or mutants with endopeptidase activity, can be identified by comparing the catalytic efficiencies against appropriate substrates, e.g. Arg-Arg-pNA, Lys-Ala-pNA, Gly-Ser-pNA, succinyl-Gly-Phe-pNA, Gly-Pro-pNA, with the catalytic efficiency of the wild type enzyme under the same conditions. Other mutants with different ratios between aminopeptidase and transferase activity with or without reduced levels of substrate restrictions are evaluated using a DPPI transferase assay. The stability of mutant forms of DPPI can be determined by e.g. incubating the mutants at elevated temperatures, in presence of chaotropic agents or detergents for the time of interest and then measure, for example, the residual aminopeptidase or transferase activity as described. DPPI mutants with reduced sensitivity to inhibition by cystatins, e.g. human cystatin C, human stefins A and B and chicken cystatin, can be identified by preincubating the mutants in presence of different levels of inhibitor and then measure the residual catalytic activity.

The invention will now be further described by way of the following non-limiting examples.

### Example 1

### Construction of Transfer Vector for Rat Prepro-DPPI

The construction of a baculovirus transfer vector termed pCLU10-4 (identical to the vector termed pVL 1393-DPPI) encoding rat DPPI preproenzyme is described in (Lauritzen et al. (1998) Protein Expr. Purif. 14, 434-442). Here, rat cDNA was prepared based on the sequence published by Ishidoh et al. (J. Biol. Chem. (1991) 266, 16312-16317). The rat prepro-DPPI encoding region was amplified by polymerase chain reaction (PCR) from the cDNA pool to generate restriction sites at the 5′ and 3′ ends of the portion of the sequence coding for the residues Met(−24)-Leu(438). Two oligonucleotide primers, 5′-GCT CTC CGG GCG CCG TCA ACC and 5′-GCT CTA GAT CTT ACA ATT TAG GAA TCG GTA TGG C (no. 6343 and no. 7436 from DNA Technology, Aahus, Denmark) were designed to specifically amplify the DNA sequence as well as to incorporate a HincII restriction site at the 5′ end and a BgIII restriction site and a TAA stop codon at the 3′ end of the coding sequence. PCR amplification was performed with these two oligonucleotide primers for 30 complete PCR cycles with each cycle involving a 1 minute denaturation step at 95° C., a 1 minute annealing step at 65° C., and a 1.5 minute polymerization step at 72° C. The cycles were followed by an extension step of 10 minutes at 72° C.

The 1395 by fragment obtained from PCR amplification and digestion with HincII and BgIII was ligated into baculovirus transfer vector pVL1393 (Catalogue #21201 P, Pharmingen, San Diego, Calif.) at the SmaI and BgIII cloning site within a multiple cloning site. The resulting transfer vector CLU10-4 also carries a strong baculovirus polyhedrin promoter, a flanking polyhedrin region from the AcNPV virus as well as an E. coli origin of replication and an ampicillin resistance gene for plasmid amplification and selection in E. coli. As cloned on pCLU10-4, the fragment encoding rat DPPI is expressed under the control of the polyhedrin promoter as prepro-DPPI i.e. with the endogenous signal sequence serving to direct secretion of rat DPPI into the culture medium. Proper vector construction was confirmed by nucleotide sequencing of the coding region on the constructed plasmid.

### Example 2

### Construction of Transfer Vector for Human Prepro-DPPI

A transfer vector termed pCLU70-1 encoding human DPPI proenzyme N-terminally fused to the signal sequence (pre-sequence) of rat DPPI preproenzyme was prepared as follows. The human pro-DPPI cDNA, previously described as a 1.9 kb full length prepro-hDPPI construct in pGEM-11Zf(−) (Paris et al. (1995) FEBS Lett. 369, 326-330) was amplified by polymerase chain reaction (PCR) to generate restriction sites at the 5′ and 3′ ends, respectively, of the portion of the hDPPI sequence coding for pro-DPPI residues-2-439 lacking all but the two N-terminal residues of the endogenous signal peptide and starting with Ser(−2) and ending with Leu(439). Two oligonucleotide primers, 5′-AAA CTG TGA GCT CCG ACA CAC CTG CCA ACT GCA-3′ (NT-HSCATC from TAGCopenhagen, Copenhagen, Denmark) and 5′-ACT GAT GCA GAT CTT TAT GAA ATA CTG GAA GGC-3′ (HS-RBGL from Gibco® BRL, LIFE TECHNOLOGIES®, Gaithersburg, Md.), were designed to specifically amplify the DNA sequence as well as incorporating a Sad restriction site at the 5′ end and maintaining a TAG stop codon and creating a BgIII restriction site at the 3′ end of the coding sequence.

PCR amplification was performed with these two oligonucleotide primers for 25 complete PCR cycles with each cycle involving a 1 minute denaturation step at 95° C., a 1 minute annealing step at 62° C., and a 1 minute polymerization step at 72° C. The cycles were followed by an extension step of 10 minutes at 72° C.

The fragment amplified from human DPPI cDNA and digested with Sad and BgIII was ligated into the baculovirus transfer vector pCLU10-4 (described in Example 1) at the SacI and BgIII sites. Thereby, the rat propPPI sequence (coding the residues (−)2-438) was deleted and replaced by the human sequence. As cloned on the resulting vector pCLU70-1, the gene fragment is expressed as a fusion between the residues 1-439 of the hDPPI sequence and the entire signal sequence for the rat DPPI protein serving to direct secretion of human DPPI into the culture medium. Proper vector construction was confirmed by nucleotide sequencing of the entire prepro-DPPI coding region on the constructed plasmid.

### Example 3

### Preparation of Recombinant Baculoviruses

For the preparation of recombinant baculoviral stocks, pCLU10-4 and pCLU70-1 were transformed into E. coli strain TOP10 (Catalogue #C4040-10, INVITROGEN®, Groningen, The Netherlands), amplified and purified by well-established methods (WIZARD® Plus SV Minipreps DNA Purification Systems, PROMEGA®, Madison, Wis.). The purified transfer vectors pCLU10-4 and pCLU70-1 were co-transfected with BaculoGold™ DNA (Catalogue #21100D, Pharmigen, San Diego, Calif.) into Spodoptera frugiperda Sf9 cells (American Type Culture Collection, Rockville, Md.) using the calcium phosphate protocol (Gruenwald et al. (1993) Procedures and Methods Manual, 2nd ed., Pharmigen, San Diego, Calif. p. 44-49). BaculoGold™ is a modified baculovirus DNA which contains a lethal deletion and accordingly cannot encode for a viable virus by itself. When co-transfected with a complementing transfer plasmid, such as pCLU10-4 or pCLU70-1, carrying the essential gene lacking in BaculoGold™, the lethal deletion is rescued and viable virus particles can be reconstituted inside transfected insect cells.

Sf9 cells were maintained and propagated at 27-28° C. as 50 ml suspension cultures in roller bottles and seeded as monolayers when used for co-transfection, plaque assays or small scale amplifications. Sf9 cells were for all purposes grown in BaculoGold™ Serum-Free medium (Catalogue #21228M, Pharmigen, San Diego, Calif.) supplemented with 5% heat inactivated foetal bovine serum (Gibco® BRL, Catalogue #10108-157). Gentamycin (Gibco® BRL, Catalogue #15750-037) to 50 mg/ml were added to cultures used for co-transfection and plaque assays.

### Example 4

### Virus Purification, Verification, and Amplification

The virus generated in the co-transfection with BaculoGold™ DNA and transfer vectors were plaque purified (Gruenwald et al. (1993) Procedures and Methods Manual, 2nd ed., Pharmigen, San Diego, Calif. p. 51-52) to generate virus particles for further infections. The structure of the purified viruses were verified by PCR. Picked plaques were suspended in 100 ml medium and incubated at 4° C. for >18 hours. 15 ml of this suspension ere used to infect High Five™ (Trichoplusia insect cells) (BTI-TN-5B1-4) (INVITROGEN®) in monolayers. High Five™ cells were maintained and propagated at 27-28° C. as 30-200 ml suspension cultures in 490 or 850 ml roller bottles in EXPRESS FIVE® SFM medium (Gibco® BRL, Cat. #10486-025), supplemented with L-Glutamine to 16.5 mM. (Gibco® BRL, Cat. #25030). 1×10⁶ cells in 2 ml medium were seeded into 6-well multidishes just before infection. The infected cells were incubated 96 hours at 27-28° C., and samples of 150 μl were taken and prepared for PCR analysis. To the 150 μl were added 350 μl H₂O, 50 μl 10% SDS and DNA was extracted from this mixture by a phenol/chloroform extraction and precipitation by ethanol and finally the DNA pellet was resuspended in 10 μl H₂O. 1 l hereof was used for PCR amplification using primers specific for the human DPPI sequence and conditions similar to the ones used for amplification of the coding regions of DPPI (Example 1 and 2). When the PCR product was analyzed on an agarose gel, a band of the expected size was obtained. Samples from cells infected with wild type AcNPV did not show this band. Recombinant viruses were also analysed for their ability to mediate expression of active DPPI. For this purpose, samples of culture medium from the infected High Five™ cells described immediately above were taken 120 hours post infection and tested using the assay as described in Example 7. When isolates were selected after the PCR analysis and the activity analysis, master virus stocks were prepared by a subsequent amplification of the plaque eluates on Sf9 cells in monolayer (Gruenwald et al. (1993) Procedures and Methods Manual, 2nd ed., Pharmigen, San Diego, Calif. p. 52-53). High titre viral stocks (>1×10⁸ plaque forming units/ml) used for scaling up the production of prepro-DPPI were obtained by further amplification on 50 ml Sf9 cell cultures in suspension (1×10⁶ cells/ml) using a multiplicity of infection (MOI) of 0.1-0.2. Virus titres were determined by plaque assay.

### Example 5

### Expression of Extracellular DPPI in Insect Cell/Baculovirus System (BEVS)

Viral stocks of CLU10-4 and CLU70-1, prepared as described in Example 4, were used to infect suspension cultures of High Five™ cells in roller bottles in EXPRESS FIVE® SFM medium supplemented with L-Glutamine to 16.5 mM. Infection of insect host cells in different experiments were carried out at a multiplicity of infection (MOI) of 1⁻¹⁰. Cell densities at the time of infection were varied in the range of 5×10⁵ to 2×10⁶ cells/ml. Cell culturing was continued for up to 6 days and samples were collected and analyzed for DPPI activity on each day from day 2 (48 hours post infection). DPPI enzyme activity was measured in the clarified media (15,000×g, 2 minutes). Recombinant DPPI was secreted as unprocessed proenzyme and the proteolytic maturation required for activity was initiated in the medium. Activation was completed in vitro by 1-2 days of incubation at low pH but for analytical purposes, activation could also be accelerated by papain treatment as described in (Lauritzen et al. (1998) Protein Expr. Purif. 14, 434-442). 5 days post infection, recombinant DPPI levels of 0.1-1 unit/ml of culture were achieved with both the human and the rat DPPI. A typical time course of DPPI activity in the culture medium from a 150 ml High Five™. culture seeded to 1×10⁶ cells/ml and infected with CLU70-1 at an MOI of 2 is shown in the table 3 below.

TABLE 3

| | without papain | with papain |
|---|---|---|
| | activation | activation |
| 72 hours post infection (units/ml) | 0.02 | 0.26 |
| 96 hours post infection (units/ml) | 0.09 | 0.40 |
| 120 hours post infection (units/ml) | 0.543 | 0.629 |

### Example 6

### Scale-Up of Secreted Human and Rat Pro-DPPI Production

High Five™ cells grown in EXPRESS FIVE® SFM medium supplemented with L-Glutamine to 16.5 mM were used to produce secreted human and rat DPPI in 0.3-2.5 litre production scales. Approximately 1.0-1.5×10⁶ cells/ml in volumes of 150 ml per 850 ml roller bottle were infected with a viral stock of CLU70-1 or pCLU10-4 at an MOI of 1-10. The roller bottles were incubated at 27-28° C. with a speed of 12 rpm. 120 hours post infection, the medium was cleared from cells and cell debris by centrifugation at 9000 rpm, 10° C., 15 minutes.

### Example 7

### Purification of Recombinant Human and Rat DPPI

Recombinant human or rat DPPI (rhDPPI and rrDPPI, respectively), in the form of partially or fully processed enzyme, could be purified from the insect cell supernatant by ammonium sulphate fractionation followed by hydrophobic interaction chromatography, desalting and anion exchange chromatography. To the clarified supernatant from e.g. 1800 ml of CLU10-4 or CLU70-1 infected cell culture was added (NH₄)₂SO₄ to 2 M and cysteamine-HCl and EDTA to 5 mM. The pH was then adjusted to 4.5 using 1 M citric acid followed by stirring for 20 min. The resulting precipitate was removed by centrifugation and filtration. The conditioned supernatant was loaded at a flow-rate of 10-15 ml/min onto a Butyl SEPHAROSE™ FF (PHARMACIA®, Uppsala, Sweden) column (5.3 cm²×35 cm) equilibrated with 20 mM citric acid, 2 M (NH₄)₂SO₄, 100 mM NaCl, 5 mM cysteamine, 5 mM EDTA, pH 4.5. The column was washed with 100 ml equilibration buffer and rhDPPI or rrDPPI was eluted with a linear gradient of 2-0 M (NH₄)₂SO₄ in equilibration buffer over 100 ml (6.6 ml/min). Fractions containing DPPI activity were pooled and incubated at 4.quadrature.C for 18-40 hours to obtain a fully processed form (see below).

The preparation of rrDPPI or rhDPPI was then desalted on a SEPAHDEX® G-25 F (PHARMACIA®, Uppsala, Sweden) column (5.3 cm²×35 cm) equilibrated with 5 mM sodium phosphate, 1 mM EDTA, 5 mM cysteamine, pH 7.0. This buffer was also used to equilibrate a Q-SEPHAROSE™ FF (PHARMACIA®, Uppsala, Sweden) column (2 cm2×10 cm) onto which the collected G-25 F eluate was loaded at a flow rate of 3 ml/min. After washing the column, rhDPPI or rrDPPI was step-eluted with desalting buffer containing 250 mM NaCl. The enzyme preparation could finally be concentrated to 40-50 units/ml in a dialysis bag embedded in PEG 6000. Finally, the enzyme preparation was formulated by addition of 1/20 volume of 5 M NaCl and 1.35 volumes of 86-88% glycerol. All chromatographic steps were carried out at 20-25° C. and the formulated product was stored at −20° C.

DPPI eluted from the hydrofobic interaction column was in general only partially processed to the mature, active form. To complete the processing, the eluate was incubated at pH 4.5 and 4° C. for 18-40 hours to convert the immature peptides to the peptides of mature rrDPPI or rhDPPI. The proteolytic processing of the peptides was accomplished by one or more cysteine peptidases present in the eluates of the Butyl SEPHAROSE™ FF column and could be completely blocked by the addition of 1 μM E-64 cysteine peptidase inhibitor or 0.1 μM chicken cystatin. Furthermore, the rate of processing was dependent on the pH of the buffer during incubation. No conversion of the immature peptides could be observed at pH 7.0 as determined by SDS-PAGE analysis but processing was observed when incubation was performed at pH 6.5 or below. The processing proceeded at highest rate at about pH 4.5. The fully processed rhDPPI and rrDPPI were finally purified and concentrated on Q-SEPHAROSE™ FF as described above. Recombinant hDPPI was quantified using an extinction coefficient at 280 nm of 2.0.

### Example 8

### DPPI Transferase Assay

The rate of transfer of dipeptides from a donor peptide to the nucleophilic amino terminus of an acceptor peptide, the ratio of dipeptide transfer to hydrolysis and the stability of elongated peptide product to hydrolytic turnover are estimated in a transferase assay.

The assay reactions are:

H-Pro-X—NH₂+H—Y-pNA→H-Pro-X—Y-pNA+NH₃ Transferase reaction

H-Pro-X—Y-pNA+H₂O→H-Pro-X—Y—COOH+pNA Trypsin cleavage

In these reactions, X and Y are any amino acid residue with the exception of prolyl. X is preferably Phe and Y is preferably Arg or Lys and pNA is a para-nitroanilide group. H and COOH indicate unblocked peptide amino and carboxy termini, respectively. In the transferase reaction, DPPI catalyses the transpeptidation of dipeptide H-Pro-X from the peptide amide to the free amino group of residue Y. The dipeptide can not be transferred to a second H-Pro-X—NH₂ molecule because of the N-terminal Pro residue. The progress of the transpeptidation reaction is monitored in the trypsin cleavage reaction, in which produced H-Pro-X—Y-pNA tripeptide is hydrolysed following the addition of trypsin endoprotease to an aliquot of reaction mixture. Trypsin hydrolyses H-Pro-X-Arg/Lys-pNA much more rapidly than H-Arg/Lys-pNA (low aminopeptidase activity) making it possible to determine the amount of tripeptide formed. The transferase reaction is essentially stopped upon addition of trypsin because the reactants are diluted 10-fold (resulting in an approximately 100-fold lower rate) and because DPPI is unstable at pH 8.3.

The concentration of tripeptide obtained also depends on the rates of hydrolysis of the initial substrate (Hydrolysis reaction 1) and of the tripeptide (Hydrolysis reaction 2):

H-Pro-X—NH₂+H₂O→H-Pro-X—COOH+NH₃ Hydrolysis reaction 1

H-Pro-X—Y-pNA+H₂O→H-Pro-X—COOH+H—Y-pNA Hydrolysis reaction 2

The hydrolysed peptides H-Pro-X—COOH and H-Pro-X—COOH are not DPPI substrates and can no longer be used in peptide synthesis. Accordingly, the peptidase activity of DPPI degrades both the trypsin substrate (before trypsin is added to the reaction mixture) and one of its precursors.

### Experimental Details

20 μl of DPPI (1-50 U/ml) in 20 mM Tris-HCl or sodium phosphate-NaOH buffer pH 7.5 is mixed with 20 μl 20 mM dithiothreitol (DTT) and allowed to incubate for 30 min at 5-37° C., preferably 12° C. Meanwhile, 10 μl 400 mM H-Pro-X—NH₂ and 10 μl 500 mM H—Y-pNA (both in 100% dimethyl formamide) and 140 μl 1100 mM Tris-HCl or sodium phosphate-NaOH buffer, pH 7.5 are mixed and incubated at the same temperature. The transferase and hydrolysis reactions are initiated by the addition of reduced and activated DPPI to the peptide mixture (same temperature). All reaction mixtures should include a minimum of 10 mM chloride.

The progress of the reaction is followed by mixing 10 μl aliquots with 1 μM trypsin in 0.1 M Tris-HCl buffer pH 8.3 and at 5-37° C., preferably 20-37° C. A yellow colour quickly appears. After 10 min, 1000 μl of water are added and the absorbance at 405 nm is measured against an appropriate blank.

Results

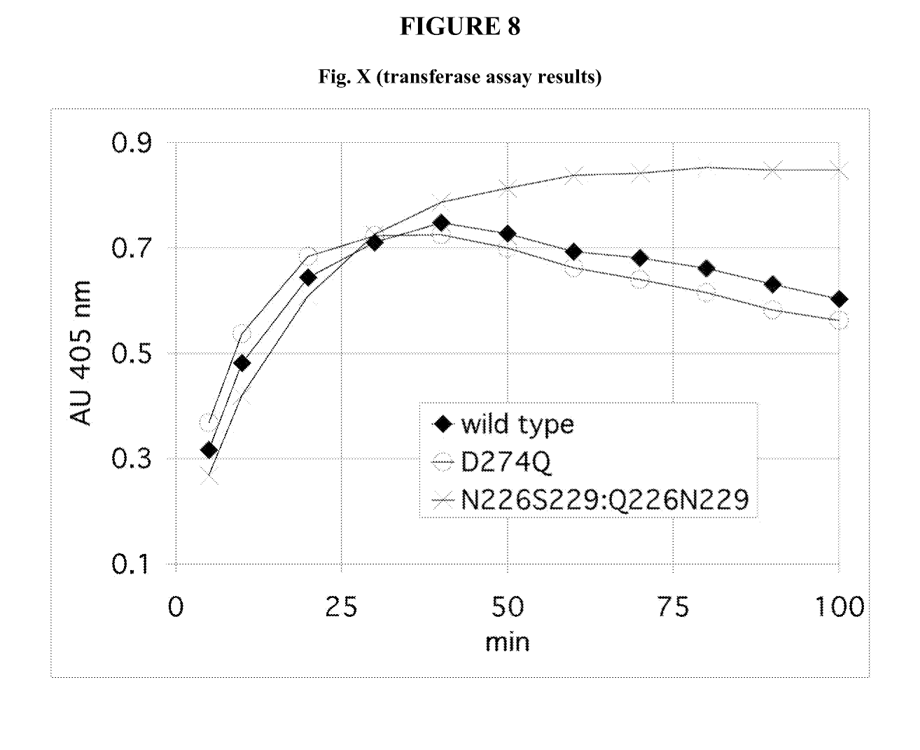

The transferase activities of wild type rat DPPI and rat DPPI mutants Asp274 to Gln274 (D274Q) and Asn226:Ser229 to Gln226:Asn229 (N226S229:Q226N229) is determined in the above transferase assay and the results are shown in FIG. 8. From the results it can be concluded that the D274Q mutation has no favourable influence on rat DPPI transferase activity. However, the N226S229:Q226N229 double mutant designed for this purpose generates the tripeptide substrate nearly as fast as the other two variants and the produced product is much more stable in presence of this rat DPPI variant. The maximum level of tripeptide also shows that the transferase activity is favoured over the hydrolytic activity.

DPPI Activity Assay

DPPI aminopeptidase activity was determined by spectrophotometrical measurement of the initial rate of hydrolysis of the chromogenic substrate Gly-Phe-p-nitroanilide (Sigma). One unit was defined as the amount of en-zyme required to convert 1 μmol of substrate per minute under the described conditions. For samples of culture medium, the assay was performed as follows: 1 part of medium was mixed with 2 parts of 200 mM cysteamine and 1 part of either water (without papain activation) or 1 mg/ml papain (with papain activation). After 10 min of incubation at 37° C., the mixture was supplemented 1:1 with fresh 200 mM cysteamine. This sample was immediately diluted 1:19 with preheated assay buffer containing the substrate (20 mM citric acid, 150 mM NaCl, 1 mM EDTA, 4 mM Gly-Phe-p-nitroanilide, pH 4.5) and the change in absorbance at 405 nm (37° C.) was measured. More concentrated samples of rDPPI and HT-rDPPI enzyme collected from steps of the purification procedure were diluted an additional 10 times with assay buffer prior to the final mixing with 200 mM cysteamine and assay buffer with substrate. The background level of hydrolysis of Gly-Phe-p-nitroanilide in the supernatant from wild-type AcNPV-cell cultures measured both with and without papain addition corresponded to 0.02 units DPPI activity per milliliter of culture. A qualitative test for DPPI activity was carried out in 96-well plates. Samples were activated with or without papain as described above. The samples and assay buffer including substrate was mixed in the wells (1:6), and the plate was incubated at 37° C. for up to 18 h and then inspected for the appearance of yellow color.

### Example 9

### Crystallization of Rat DPPI and Collection of Native and Heavy Atom Derivative X-ray Diffraction Data

The stock solution contained 1.5 mg/ml of protein as estimated by absorption at 280 nm, assuming an extinction coefficient of 1.0, in 25 mM sodium phosphate pH 7.0, 150 mM NaCl, 1 mM ethylene diamine triacetate (EDTA), 2 mM cysteamine and 50% glycerol. The solution was stored at −18° C. Prior to crystallisation, 10 ml of the stock solution was dialysed for 20 hours against 5 l of 20 mM bis-tris-HCl pH 7.0, 150 mM NaCl, 2 mM dithiothreitol (DTT), 2 mM EDTA. Dialysis was performed against two times 2 litres (4 and 18 h, respectively) with no apparent difference in behaviour of the enzyme preparation. The protein was concentrated to 16.1 mg/ml and a fast screen was set up (HAMPTON CRYSTAL SCREEN™ I). The hanging drop vapour diffusion technique was employed with 0.8 ml reservoir solution and drops containing 2 μl protein solution and 2 μl reservoir solution.

Crystals appeared after 30 min in condition 4 (0.1 M Tris pH 8.5, 2.0 M (NH₄)₂SO₄). Crystals grew from conditions 4, 6, 17, 18, and 46. Incubation under conditions 4, 6 and 17 resulted in the formation of star-shaped crystals whereas conditions 18 and 46 resulted in box-shaped crystals.

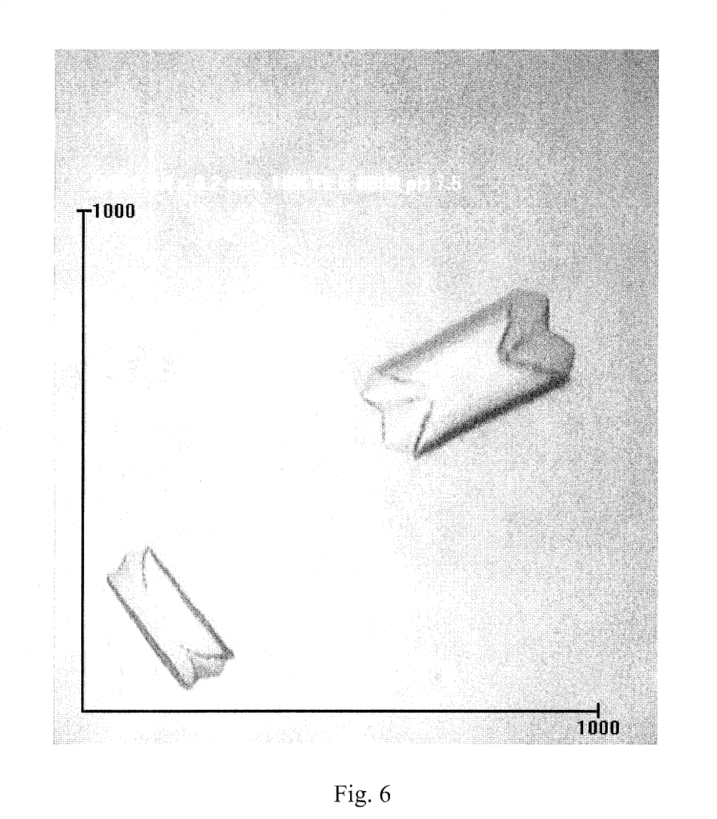

Optimisations using incomplete factorial design experiments showed an optimum for the box shaped crystal form using reservoir solution containing 0.1 M bis-tris propane pH 7.5, 0.15 M calcium acetate and 10% PEG 8000. Drops were set up with equal volumes of reservoir solution and protein solution. The protein concentration was 12 mg/ml. A representative crystal is shown in FIG. 6. The box-shaped crystals diffracted very poorly (out to 5 Å resolution at best).

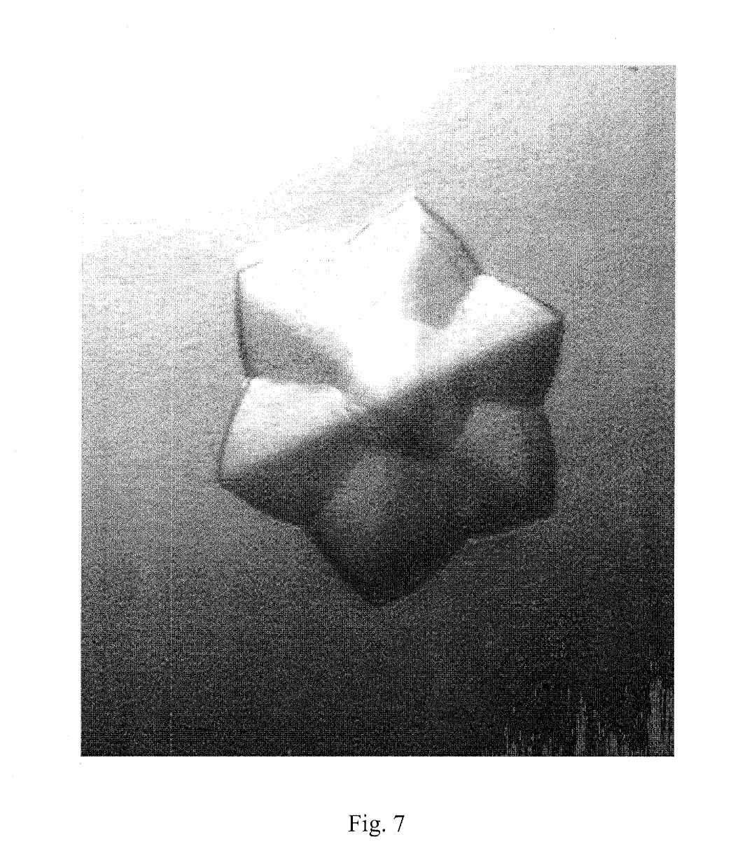

Optimum crystallisation conditions for the star-shaped crystal form were fairly close to the fast screen conditions and at 1.4 M (NH₄)₂SO₄ and 0.1 M bis-tris propane pH 7.5, each drop contained one to three well defined crystals. The maximum length (the ‘diameter’) varied between 0.5 and 1 mm, the thickness varied between 0.1 and 0.4 mm at the centre. A representative crystal is shown in FIG. 7. These crystals diffracted to between 4 and 5 Å resolution on rotating anode equipment and to 3 Å resolution using synchrotron radiation at .div.10° C. When cryo conditions were found and the crystals could be cooled to 110 K, they diffracted to 2.4 Å resolution (see the following section).

Initial diffraction experiments were performed on the RAXIS II imaging plate detector using CuK.alpha. radiation from a rotating anode operated at 50 kV, 180 mA. Diffraction was never detected beyond 4.2 Å under these conditions. Therefore, the crystals were taken to the MAX LAB synchrotron facility in Lund, Sweden. Unfortunately, cooling the crystals to 110 K using glycerol or glucose as a cryo protectant did not improve the diffraction power. Furthermore, the cryo protectant quite often ruined the crystal completely. The use of PEG destroyed the crystals instantaneously. For the collection of derivative data (see below), glycerol was most often used as a cryo protectant based on the observation that crystals incubated with glycerol survived for longer periods of time (over night), as determined by visual inspection, than did crystals incubated with glucose (visible damage after 2 h). It was also possible to cool down the crystals taken directly from the mother liquor to −15° C. in a capillary without ice formation because of the high (NH₄)₂SO₄ content. The space group was determined to be hexagonal based on auto indexing in the program DENZO® (Otwinowski, Z, Minor, W. (1997) Methods Enzymol. 276A, 307-326). Processing the data in P6 with SCALEPACK (Otwinowski, Z, Minor, W. (1997) Methods Enzymol. 276A, 307-326) and searching for systematic absences in hklview from the CCP4 program suite (Collaborative Computational Project, Number 4 (1994) Acta Crystallogr. D 50, 760-763) gave the symmetry along the axes and the space group was determined to be either P6422. The unit cell dimensions are a=166.24 Å, b=166.24 Å, c=80.48 Å, α=90°, β=90°, γ=120°

This rather large unit cell gave rise to a very dense diffraction pattern which introduced the danger of overlap between reflections. This can be overcome in several ways: 1) By moving the detector away from the crystal since the divergence of the diffracted beams relative to each other is larger than the divergence of the individual beams because the X-ray beam is focused; 2) By collecting with fine .o slashed. slicing, i.e. by oscillating over a very narrow angular space (<10) such that the reflections recorded only represent a very narrow ‘slice’ of reciprocal space; 3) By orienting the crystal such that a full data set is recorded with as few images as possible being recorded while the incoming beam is parallel to a long unit cell axis; 4) By ensuring that the beam is well focused and that the cross section of the beam is of the same size as that of the crystal; 5) By optimising the cryo conditions to reduce mosaicity. Depending on the crystal and equipment, only some of these options may be open to the experimenter. In the case of cathepsin C crystals, the derivative data sets and the first native data set were recorded at −10° C. At such high temperatures, there is extensive radiation damage to the crystal and as completeness of the data is of primary concern, the fine 0 slicing method is not an option. Under these conditions, the crystals only diffracted to a maximum of 3 Å so the detector can be moved far away from the crystal but also here, this must be balanced since the diffracted beams lose intensity as a function of the distance they travel through air. By fine tuning the experiment, it was possible to obtain relatively good data from the cathepsin C crystals at −10° C. However, they suffered from rather poor resolution (between 3 and 4 Å) and incompleteness.

Following fine tuning the experimental conditions, it was possible to record an incomplete data set to 3-4 Å resolution at −10° C.

Optimisation of Cry Conditions

Encouraged by the work by Garman (Garman, E. (1999) Acta Crystallogr. D 55, 1641-1653), a search for new cryo conditions was initiated. Soaking the rat DPPI crystals with glucose seemed to give slightly better results with respect to diffraction, pointing out the fact that the visual damage to the crystal as a result of prolonged incubation with the cryo protectant (described above) is perhaps not a good parameter for determining the proper cryo solution. The following experiment was then carried out: a series of reservoir solutions containing from 6% to 34% sucrose in steps of 2%-points, except the last step which was 8%-points, was prepared. A crystal was carefully transferred with a cryo loop from the mother liquor to the first drop where it rested for 1 minute, then on to the next for 1 minute and so on. Crystal mounting took approximately 3-4 seconds and was performed by blocking the cryo stream (N₂ gas at 110 K) with a credit card, positioning the loop on the goniometer head and removing the card. Several crystals were tested. The largest crystals seemed to exhibit slightly higher mosaicity. Crystals with a diameter of 0.5 mm gave the best results which is probably because the larger ones takes a significant time in the stream before the core reaches the same temperature as the surface. Using crystals with a diameter of 0.5 mm, a complete data set to 2.4 Å resolution and with high redundancy was collected (see Table 1.1). The structure at 2.4 Å has currently been refined to R=0.247, Rfree=0.282.

TABLE 1.1
Data collection details and statistics for the native
dataset used to solve the structure of rat DPPI.
Data collection and statistics

| | |
|---|---|
| Crystal to detector distance (mm) | 255 |
| Δφ (°) | 1 |
| Angular space covered (°) | 132 |
| λ (Å) | 0.984 |
| Resolution range | 30.0-2.4 |
| Completeness (%) | 99.2 |
| Number of reflections | 741631 |
| Unique reflections | 25816 |
| R_{sym} (%) | 7.1/32.2 |
| R_{merge} (%) | 8.1 |

Data were collected at the MAX Lab synchrotron, beam line 711.

Determining the Phases by Multiple Isomorphous Replacement (MIR)

The phases for the structure factor amplitudes calculated from the X-ray diffraction pattern from crystals of rat DPPI were determined by the method of multiple isomorphous replacement (Blundell, T. L., Johnson, N. L. (1976) Protein Crystallography, Academic Press). A major problem concerning the initial experimental work on DPPI crystals was the lack of cryo conditions combined with poor X-ray diffraction. This necessitated high radiation dosage and thus the crystals rapidly lost diffraction power during X-ray exposure because of the radiation damage, especially when using synchrotron radiation. It was not possible to record complete data sets. Incompleteness of a derivative data set is in principle not very serious once the heavy atom positions have been determined since from that point on, everything is calculated in reciprocal space and the phase extension functions very efficiently fill in the gaps. Needless to say, completeness of the native data set is important. Unfortunately, the method used at the time to solve the phase problem of DPPI was the difference Patterson method. Incompleteness of derivative data can be a problem if the derivative is weak, i.e. low occupancy or if there is noise due to non-isomorphism, since the missing reflections are set to zero for the difference Patterson calculation which is presumably a poor estimate. Three derivative data were analysed. These were mercury acetate (Hg-acetate), dipotassium tetrachloro aurate (K₂AuCl₄), and para-hydroxy mercuribenzoic acid (PHMBA). Laborious attempts to solve the difference Patterson maps were undertaken. Sites were obtained which gave even poorer phasing statistics than the ones shown in Table 1.2 because the sites were imprecisely determined due to noise and the co-ordinate refinement in the CCP4 program mlphare (number 4, 1991) used did not refine co-ordinates sufficiently. Furthermore, the difference in statistics between invented sites (i.e. sites with random co-ordinates) and sites deduced from the difference Patterson maps were very small although the phasing power of ‘real’ sites was consistently slightly higher, and adding ‘real’ sites to the refinement gave increased figures of merit. A heavy atom site search was performed using a modified version of the molecular replacement program AMoRe (Navaza, J. (1994) Acta Crystallogr. A 50, 157-163), called HAMoRe (Anders Kadziola). AMoRe performs a real space rotation search (Navaza, J. (1993) Acta Crystallogr. D 49, 588-591) and a reciprocal space translation search (Navaza, J., Vernoslova, E. (1995) Acta Crystallogr. A 51, 445-449). Assuming that the heavy atom peaks are spherical, there is no need for a rotation search and so the calculation can be restricted to reciprocal space thus avoiding the noise in the difference Patterson map introduced by the missing reflections. The method is very reliable and has been implemented for heavy atom searching in CNS program (Brunger, A. T., Adams, P. D., Clore, G. M., DeLano, W. L., Gros, P., Grosse-Kunstleve, R. W., Jiang, J. S., Kuszewski, J., Nilges, M., Pannu, N. S., Read, R. J., Rice, L. M., Simonson, T., Warren, G. L. (1998) Acta Crystallogr. D 54, 905-921). The HAMoRe fast translation function search found 2 sites in each derivative data set. Each site was systematically omitted and validated by difference searches using the phase information from the other sites. These six sites were scaled against the native data set, refined and phases were calculated for the native data set between 8 and 3.5 Å (Table 1.2). As can be seen, the phasing power and R_{cullis} values for these sites were relatively low. Combining the sites in mlphare gave an overall figure of merit of 0.491 and after solvent fattening and histogram matching using dm (Cowtan, K., Main, P. (1998) Acta Crystallogr. D 54, 487493) from the CCP4 suite, this value increased to 0.610.

TABLE 1.2
Data collection and phasing statistics of heavy atom
derivatives of rat cathepsin C crystals.

| Data set | HgCl₂ | K₂AuCl₄ | PHMBA |
|---|---|---|---|
| Number of unique reflections | 6204 | 6523 | 5681 |
| Completeness (%) | 72 | 75 | 66 |
| Resolution (Å) | 15.0-3.3 | 15.0-3.2 | 15.0-3.3 |
| Weighted Rᵢₛₒ^{a} (15-3.5 Å) | 0.504 | 0.512 | 0.483 |
| Number of sites used for phasing | 2 | 2 | 2 |
| Figure of merit^{b} | 0.30 | 0.31 | 0.27 |
| Phasing power^{c} | 1.18 | 1.08 | 1.18 |
| R_{cullis}^{d} | 0.81 | 0.85 | 0.81 |

PHMBS = para-hydroxy mercurybenzoic acid. Lack of closure analysis using means. Acentric reflections only.
^{a}Rᵢₛₒ = Σhkl|F_{der} − Fₙₐₜ|/Σ|Fₙₐₜ|.
^{b}The figure of merit, m = | Fₕₖₗ(best)|/|Fₕₖₗ|, such that Fₕₖₗ(best) = |Fₕₖₗ|m exp [iα(best)], where α(best) is centroid of the phase angle probability distribution.
^{c}The phasing power is the root mean square of Fₕ/E where Fₕ is the structure factor for the heavy atom contribution and E is the residual lack of closure.
^{d}R_{cullis} = Σ|F_{h(obs)} − F_{h(calc)}|/ΣF_{h(obs)}.

Attempting at this stage to extend the phases all the way to 2.4 Å gave figures of merit below 0.3 for extended phases. This extended map was better than the non-extended as determined by visual inspection. Yet, the map could not readily be interpreted. Using the phases after density modification as input in miphare along with the refined heavy atom sites to aid the refinement and precision of phasing gave a mean figure of merit of 0.926 for all reflections to 3.5 Å (miphare output) and after phase extension to 2.4 Å, in dm, the mean figure of merit was 0.567 for reflections to 2.4 Å. This map was much nicer but exhibited streaking in the z-direction hampering model building. By dividing the data set in resolution shells and plotting the strongest reflection for each bin an outlier was detected around 4.5 Å resolution (hkl=(36, 10, 1)). This outlier was excluded and the streaking disappeared. The map was now interpretable. Although the papain core domain part of the protein was modelled into the density and this constitutes half or more of the entire structure, model phases were avoided for phasing because of the danger of model bias. Combining experimental phases with model phases (using CCP4 programs sfall and sigmaa) did in fact give alarmingly nice density around the model without improving the map outside the model.

### Example 10

### Design and Construction of Rat DPPI Active Site Mutant Asp274 to Gln274

From investigations of the three dimensional structure of rat DPPI, it can be concluded that Asp274 (pro-DPPI numbering) is one of the only charged residues located in the active site of rDPPI, which get in close proximity to the two N-terminal residues that dock into the S₁ and S₂ substrate binding pockets upon successful binding of an appropriate peptide substrate into the active site cleft of rDPPI. Mutation of this residue may effect the catalytic function of the enzyme, in particular with respect to hydrolysing peptide substrates having lysine or arginine residues located in the penultimate position (second residue from the N-terminus; peptides with N-terminal lysine or arginine residues are not substrates) as these basic residues may interact favourably with the negative charge on Asp274 in the wild type enzyme. Removing the negative charge on Asp274 may thus change the specificity of the enzyme.

Because of the large size of those lysine and arginine residue side chains that may interact favourably with Asp274, one can chose to mutate Asp274 to a glutamine residue. A Gln residue is selected because it is uncharged, has a structure comparable to Asp, is able to function as both a hydrogen bond donor and acceptor and is slightly longer than Asp thereby potentially compensating for shorter lengths of penultimate substrate residue side chains.

To perform site-directed mutagenesis of rat DPPI residue Asp274 into glutamine, according to the method of Nelson and Long (1989) (Nelson, R. M. and Long, G. L. (1989) A general method of site-specific mutagenesis using a modification of the Thermus aquaticus polymerase chain reaction. Anal. Biochem. 180, 147-51), the degenerate reverse oligonucleotide MRI (5′-TGG GAA TCC ACC TT(G/C) ACA ACC TTG GGC-3′), encoding either Gln or Glu in position 274, is used. First, cDNA encoding wild type rat prepro-DPPI (contained in baculovirus transfer vector pCLU10-4, stock #30) is amplified in a polymerase chain reaction (PCR) using the MR1 oligonucleotide and a hybrid forward oligonucleotide, HF1 (5′-CGG GCT GAC TAA CGG CGG GGC AAT TTT GTT AGC CCT GTT CG-3′). The 3′ end of HF1 anneals upstream of a unique EcoRI site in the cDNA (see FIG. 1) whereas the 5′ end of HF1 has the same sequence as the oligonucleotide H5′ (5′-CGG GCT GAC TAA CGG CGG GG-3′). Following amplification and purification of the product (201 bp, all fragment sizes are approximate), the amplified fragment is annealed to the same wild type rat prepro-DPPI template and extended towards the 3′ end of the cDNA in 2 PCR amplification cycles. Hereafter, the temperature of the reaction mixture is maintained at 85° C. while the forward H5′ oligonucleotide and the reverse oligonucleotide R2 (5′-GTG TCG GGT TTA ACA TTA CG-3′), which anneals downstream of a unique 3′ Bg/II restriction site, are added. Following the addition of oligonucleotides, a second round of PCR amplification is performed. The produced fragment of 763 by carries the unique EcoRI and Bg/II sites close to its termini, and after EcoRI and Bg/II digestion of both this fragment and of the vector and de-phosphorylation of the vector ends using alkaline phosphatase (calf intestinal), the PCR amplified EcoRI-Bg/II fragment of 583 by is ligated into the vector. Following transformation and isolation of pure clones, bacterial colonies carrying the desired transfer vectors, with a single mutagenised codon encoding either a glutamine or a glutamate residue in position 274, is identified by DNA sequencing.

Experimental Conditions

Purification of Transfer Vector pCLU 10-4

Vector pCLU10-4 is purified from a bacterial culture of transformed TOP10 cells by JETStar midi-prep, ethanol/ammonium acetate precipitation, washing in 70% ice-cold ethanol and redissolution in 1:1 (v/v) mixture of demineralised water and 10 mM TB buffer (pH 8.0). The concentration of plasmid is approximately 0.3 μg/μl as estimated by agarose gel electrophoresis and comparison of the ethidium bromide staining intensity with those of DNA fragment size marker bands (HindIII digested lambda-phage DNA).

EcoRI/BgIII Restriction Digestion of Transfer Vector pCLU10-4

In an EPPENDORF® reaction tube, the following chemicals are mixed:

| | |
|---|---|
| Transfer vector pCLU10-4 | 30.0 μl |
| EcoR1 (25 U/μl, Pharmacia ®) | 0.35 μl |
| BglII (25 U/μl, Pharmacia ®) | 0.60 μl |
| 10c React 3 burrer (LIFE TECHNOLOGIES ®) | 3.5 μl |

Incubation at 37° C. for 30 min

| | |
|---|---|
| Alkaline phosphatase (1 U/μl, Pharmacia ®) | 0.2 μl |

Incubation at 37° C. for 30 min

The cleavage reaction is purified by preparative agarose gel electrophoresis and the excised EcoRI-Bg/II fragment can be observed in the gel (583 bp). The vector of 10.408 by is recovered from the gel by freezing and thawing of the gel portion containing the vector, centrifugation of the gel portion (10,000 rpm/10 min) in a COSTAR® SPIN-X® centrifuge tube (catalogue #8162), equipped with a 0.22 μm cellulose acetate filter that withholds the denatured agarose but not buffer or DNA, and ethanol/ammonium acetate precipitation of the flow-through. The precipitated vector is washed and redissolved in 50 μl of water.

Amplification of Transfer Vector pCLU10-4 Using HF1 and MR1 Oligonucleotides

| | | |
|---|---|---|
| Transfer vector pCLU10-4 (XhoI digest) | 0.5 | μl |
| 10x AmpliTaq reaction buffer (Perkin Elmer) | 10 | μl |
| 25 mM MgCl₂ (C^{Mg2+}_{final} = 1.5 mM) | 6 | μl |
| 4 × 5 mM dNTP | 4 | μl |
| HF1 (50 μM) | 2 | μl |
| MR1 (50 μM) | 2 | μl |
| Demineralised water | 76 | μl |

Incubation at 95° C. for (5′:00)
Temperature shift to 85° C. (5′:00″)

| | | |
|---|---|---|
| Addition AmpliTaq DNA polymerase (5 U/μl) | 0.5 | μl |
| Oil overlay | | |

15 PCR cycles:
95° C. (1′:00″) then 50° C. (1′:00″) then 72° C. (0′:30″) [repeated]
72° C. (10′:00″) then 4° C. (hold)

The amplified fragment (201 bp) is purified by 1.5% agarose gel electrophoresis, freezing and thawing and centrifugation in COSTAR® SPIN-X® columns.

Elongation and Amplification of HF1:MR1Product

| | | |
|---|---|---|
| Transfer vector pCLU10-4 (XhoI digest) | 0.5 | μl |
| 10x AmpliTaq reaction buffer (Perkin Elmer) | 10 | μl |
| 25 mM MgCl₂ (C^{Mg2+}_{final} = 1.5 mM) | 6 | μl |
| 4 × 5 mM dNTP | 4 | μl |
| Purified HF1: MR1 amplification product | 2 | μl |
| Demineralised water | 74 | μl |

Incubation at 95° C. for (5′:00)
Temperature shin to 85° C. (5′:00″)

| | | |
|---|---|---|
| Addition AmpliTaq DNA polymerase (5 U/μl) | 0.5 | μl |
| Oil overlay | | |

2 PCR cycles:
95° C. (1′:00″) then 50° C. (2′:00″) then 72° C. (5′:00″) [repeated]
Addition of oligonucleotide after 1′:30″ of the second 72° C.
incubation:

| | | |
|---|---|---|
| H5′ (50 μM) | 2 | μl |
| R2 (50 μM) | 2 | μl |

15 PCR cycles:
95° C. (1′:00″) then 60° C. (1′:00″) then 72° C. (10′:00″) [repeated]
72° C. (10′:00″) then 4° C. (hold)

The amplified fragment is purified by 1.5% agarose gel electrophoresis, freezing and thawing and centrifugation in COSTAR® SPIN-X® columns. The fragment is further purified using the QIAQUICK® PCR purification kit (QIAGEN®, catalogue #28106).

EcoRI/BgIII Restriction Digest of H5′:R2PCR Product

In an EPPENDORF® reaction tube, the following chemicals are mixed:

| | |
|---|---|
| H5′:R2 PCR product | 25.0 μl |
| EcoRI (25 U/μl, Pharmacia) | 1.4 μl |
| BglII(15 U/μl, Pharmacia) | 1.7 μl |
| 10x React 3 buffer (Life Technologies) | 3.3 μl |

Incubation at 37° C. for 1 hr

30 μl cleavage reaction mixture is subjected to preparative agarose gel electrophoresis and the purified product is recovered using SPINX® and QIAQUICK® spin columns as described. The final elution volume is 40 μl.

Ligation of EcoRI:Bg/II Cut pCLU10-4 Vector and H5′:R2 Fragment

| | |
|---|---|
| EcoRI:BglII cut pCLU10-4 | 2 μl |
| EcoRI:BglII cut H5′:R2 fragment | 6 μl |
| 10x All-for-One⁺ buffer (Pharmacia) | 1 μl |
| 10 mM ATP | 1 μl |
| T4 DNA ligase | 0.5 μl |

Incubation at 16° C. for 2 hrs
Incubation at 4° C. overnight

The ligated vector is transformed into electrocompetent E. coli TOP10 cells using a BTX E. coli TransPorator™ charged with 1.500 V (1 mm cell width). Transformed cells are reconstituted in SOC medium and purified and identified by plating on agar plates containing 100 μg/ml ampicillin. Incubation at 37° C. for 15-20 hrs. Clones carrying vectors with the desired sequence is identified by DNA sequencing of purified plasmid DNA using e.g. the R2 oligonucleotide as a primer in the sequencing reaction. The described methods and the technique of DNA sequencing are well known to people skilled in the arts.

### Example 11

### Design and Construction of Rat DPPI Active Site Mutant Asn226:Ser229 to Gln226:Asn229

From investigations of the three dimensional structure of rat DPPI, residues Asn226 and Ser229 (pro-DPPI numbering) are selected for mutation to increase the affinity of the active site cleft prime-site substrate binding sites (sites that bind substrate residues C-terminal of the cleavage site) for peptide substrates. Following formation of the thio-ester bond in the first step of catalysis (see reaction scheme 1#, step 1), a stronger binding of peptides to the prime-site substrate binding region is suggested to favour liberation of the bound N-terminal portion of the substrate by aminolysis (step 2, aminolysis) and potentially reduce hydrolysis (step 2, hydrolysis) as a result of steric hindrance of water molecules by the bound peptides. In the reaction scheme, Pₓ and P_{y} represent substrate residues located N- and C-terminal of the cleavage site, respectively, HS-Cys233 is the catalytic cysteine in the enzyme E and Xₙ are residues in the acceptor peptide that causes aminolysis.

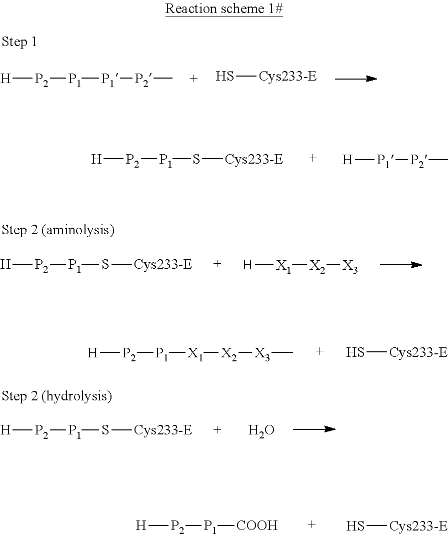

The mutation of Asn226 and Ser229 into Gln and Asn, respectively, may enhance peptide binding by having longer side chains that can participate in hydrogen bond formation, both as donors and acceptors. In the structure of rat DPPI, it can be seen that the side chains of Asn226 and Ser229 may be too short to strongly interact with peptide substrates.

Experimental Conditions

To perform site-directed mutagenesis of rat DPPI residue Asn226 and Ser229 into Gln226 and Asn229, according to the method of Nelson and Long (1989) (Nelson, R. M. and Long, G. L. (1989) A general method of site-specific mutagenesis using a modification of the Thermus aquaticus polymerase chain reaction. Anal. Biochem. 180, 147-51), the degenerate reverse oligonucleotide MR1 (5′-TGG GAA TCC ACC TT(G/C) ACA ACC TTG GGC-3′), the degenerate forward oligonucleotide MF5 (5′-TAG CCC TGT TCG ACA ACA AGA A(A/G)A TTG TGG AAG CTG C-3′), encoding Gln in position 226 and either Asn or Asp in position 229, is used. First, cDNA encoding wild type rat prepro-DPPI (contained in baculovirus transfer vector pCLU10-4, stock #30) is amplified in a polymerase chain reaction (PCR) using the MF5 oligonucleotide and a hybrid reverse oligonucleotide, HR2 (5′-CGG GCT GAC TAA CGG CGG GGG GCA ACT GCC ATG GGT CCG-3′). The 3′ end of HR2 anneals downstream of a unique EcoRI site in the cDNA (see FIG. 1) whereas the 5′ end of HR2 has the same sequence as the oligonucleotide H5′ (5′-CGG GCT GAC TAA CGG CGG GG-3′). Following amplification and purification of the product (402 bp), the amplified fragment is annealed to the same wild type rat prepro-DPPI template and extended towards the 5′ end of the cDNA in 3 PCR amplification cycles. Hereafter, the temperature of the reaction mixture is maintained at 85° C. while the reverse H5′ oligonucleotide and the forward oligonucleotide F1 (5′-CGG ATT ATT CAT ACC GTC CC-3′), which anneals upstream of a unique 5′ SacI restriction site, are added. Following the addition of oligonucleotides, a second round of PCR amplification is performed. The produced fragment of (1179 bp) carries the unique Sad and EcoRI sites in its termini, and after Sad and EcoRI digestion of both this fragment and of the vector and de-phosphorylation of the vector ends using alkaline phosphatase (calf intestinal), the PCR amplified SacI-EcoRI fragment of 740 by is ligated into the vector. Following transformation and isolation of pure clones, bacterial colonies carrying the desired transfer vectors, with a single mutagenised codon encoding either a asparagine or a aspartate residue in position 229, is identified by DNA sequencing.

SacI/EcoRI Restriction Digestion of Transfer Vector pCLU10-4

In an EPPENDORF® reaction tube, the following chemicals are mixed:

| | |
|---|---|
| Transfer vector pCLU10-4 (prepared as described) | 25.0 μl |
| SacI (15 U/μl, Pharmacia) | 2.0 μl |
| EcoRI (25 U/μl, Pharmacia) | 1.2 μl |
| 10x One-Phor-All⁺ buffer (Pharmacia) | 4.0 μl |
| Demineralised water | 8.0 μl |

Incubation at 37° C. for 40 mm

| | |
|---|---|
| Alkaline phosphatase (1 U/μl, Pharmacia) | 0.5 μl |

Incubation at 37° C. for 35 min

The cleavage reaction is purified by preparative agarose gel electrophoresis and the excised SacI-EcoRI fragment can be observed in the gel (740 bp). The vector of 10.251 by is recovered from the gel portion by freezing and thawing of the gel portion containing the vector, centrifugation of the gel (10,000 rpm/10 min) in a COSTA® SPIN-X® centrifuge tube (catalogue #8162), equipped with a 0.22 μm cellulose acetate filter that withholds the denatured agarose but not buffer or DNA, and ethanol/ammonium acetate precipitation of the flow-through. The precipitated vector is washed and redissolved in 50 μl of water.

Amplification of Transfer Vector pCLU10-4 Using MF5 and HR2 Oligonucleotides

| | | |
|---|---|---|
| Transfer vector pCLU10-4 (XhoI digest) | 0.5 | μl |
| 10x AmpliTaq reaction buffer (Perkin Elmer) | 10 | μl |
| 25 mM MgCl₂ (C^{Mg2+}_{final} = 1.5 mM) | 6 | μl |
| 4 × 5 mM dNTP | 4 | μl |
| MF5 (50 μM) | 2 | μl |
| HR2 (50 μM) | 2 | μl |
| Demineralised water | 76 | μl |

Incubation at 95° C. for (5′:00)
Temperature shift to 85° C. (5′:00″)

| | | |
|---|---|---|
| Addition AmpliTaq DNA polymerase (5 U/μl) | 0.5 | μl |
| Oil overlay | | |

15 PCR cycles:
95° C. (1′:00″) then 50° C. (1′:00″) then 72° C. (0′:30″) [repeated]
72° C. (10′:00″) then 4° C. (hold)

The amplified fragment (402 bp) is purified by 1.5% agarose gel electrophoresis, freezing and thawing and centrifugation in COSTA® SPIN-X® columns.

Elongation and Amplification of MF5:HR2 Product

| | | |
|---|---|---|
| Transfer vector pCLU10-4 (XhoI digest) | 0.5 | μl |
| 10x AmpliTaq reaction buffer (Perkin Elmer) | 10 | μl |
| 25 mM MgCl₂ (C^{Mg2+}_{final} = 1.5 mM) | 6 | μl |
| 4 × 5 mM dNTP | 4 | μl |
| Purified MF5: HR2 amplification product | 10 | μl |
| Demineralised water | 65 | μl |

Incubation at 95° C. for (2′:00)
Temperature shift to 85° C. (5′:00″)

| | | |
|---|---|---|
| Addition AmpliTaq DNA polymerase (5 U/μl) | 0.5 | μl |
| Oil overlay | | |

3 PCR cycles:
95° C. (1′:00″) then 50° C. (2′:00″) then 72° C. (5′:00″) [repeated]
Addition of oligonucleotide after 1′:30″ of the second 72° C.
incubation:

| | | |
|---|---|---|
| H5′ (50 μM) | 2 | μl |
| F1 (50 μM) | 2 | μl |

20 PCR cycles:
95° C. (1′:00″) then 60° C. (1′:00″) then 72° C. (10′:00″) [repeated]
72° C. (10′:00″) then 4° C. (hold)

The amplified fragment is purified using the QIAQUICK® PCR purification kit (QIAGEN®, catalogue #28106). The product is eluted in 50 μl TE buffer.

SacI/EcoRI Restriction Digest of F1:H5′ PCR Product

In an EPPENDORF® reaction tube, the following chemicals are mixed:

| | |
|---|---|
| F1:H5′ PCR product | 48.0 μl |
| SacI (15 U/μl, Pharmacia) | 2.0 μl |
| EcoRI (25 U/μl, Pharmacia) | 1.2 μl |
| 10x All-for-One⁺ buffer (Pharmacia) | 5.5 μl |

Incubation at 37° C. for 1 hr

The cleavage reaction mixture is subjected to preparative agarose gel electrophoresis and the purified product is excised and recovered using SPIN-X® and QIAQUICK® spin columns as described.

Ligation of SacI:EcoRI Cut pCLU10-4 Vector and F1:H5′ Fragment

| | |
|---|---|
| SacI:EcoRI cut and dephos. pCLU10-4 vector | 8 μl |
| SacI:EcoRI cut H5′:R2 fragment | 9 μl |
| 10x All-for-One⁺ buffer (Pharmacia) | 1 μl |
| 10 mM ATP | 2 μl |
| T4 DNA ligase | 0.5 μl |

Incubation at 16° C. for 2 hrs
Incubation at 4° C. overnight

The ligated vector is Ethanol/ammonium acetate precipitated, washed in 70% ethanol and redissolved in 5 μl TE buffer. 1 μl of this plasmid is used to transform electrocompetent E. coli DH10B cells using a BTX E. coli TransPorator™ charged with 1.500 V (1 mm cell width). Transformed cells are reconstituted in SOC medium and purified and identified by plating on agar plates containing 100 μg/ml ampicillin. Incubation at 37° C. for 15-20 hrs. Clones carrying vectors with the desired sequence is identified by DNA sequencing of purified plasmid DNA using e.g. the F1 oligonucleotide as a primer in the sequencing reaction. The described methods and the technique of DNA sequencing are well known to people skilled in the arts.

### Example 12

The crystal structure of human DPPI.

The structural co-ordinates are shown in table 2b.

Overall structure: Tetrahedron is dimer of dimers.

The tetrameric molecule of DPPI has a shape of a slightly flattened sphere with a diameter of approximately 80 Å and a spherical cavity with a diameter of about 20 Å in the middle. The molecule has tetrahedral symmetry. The molecular symmetry axis coincides with the crystal symmetry axis of the 1222 space group. The asymmetric unit of the crystal thus contains a monomer. Each monomer consists of three domains, the two domains of the papain-like structure containing the catalytic site, and an additional domain. This additional domain with no analogy within the family of papain-like proteases contributes to the tetrahedral structure and creates an extension of the active site cleft providing features which endow DPPI with amino-dipeptidyl peptidase activity (FIG. 10). We term this additional domain the “residual propart” domain (Dahl et al., 2001).

The residues of a monomer are numbered consecutively according to the zymogen sequence (Paris et al., 1995). The observed crystal structure of the mature enzyme contains 119 residues of the residual propart domain from Asp 1 to Gly 119 and 233 residues of the two papain-like domains from Leu 207 to Leu 439. The papain-like structure is composed of N-terminal heavy and C-terminal light chains generated by cleavage of the peptide bond between Arg 370 and Asp 371. The 87 propeptide residues from Thr 120 to His 206, absent in the mature enzyme structure, were removed during proteolytic activation of the proenzyme. The structure confirms the cDNA sequence (Paris et al., 1995) and is in agreement with the amino acid sequence of the mature enzyme (Cigic et al., 1998; Dahl et al., 2001). With the exception of Arg 26, all residues are well resolved in the final 2fo-fc electron density map. The conformations of the regions Asp 27-Asn 29 within the residual propart domain and Gly 317-Arg 320 at the C-terminus of the heavy chain are partially ambiguous.

During activation, the structure of DPPI undergoes a series of transformations. From the presumably monomeric form of preproenzyme (Muno et al., 1993), via a dimeric form of proenzyme (Dahl et al., 2001), the tetrameric form of the mature human enzyme is assembled (Dolenc et al., 1995). Visual inspection along each of the three molecular twofold axes showed that one of the axes reveals a head-to-tail arrangement of a pair of papain-like and residual propart domains (FIG. 10b). The N-terminus of the residual propart domain of one dimer binds into the active site cleft of the papain-like domain of the next, while the C-terminus of one papain-like domain binds into the beta-barrel groove of the adjacent residual propart domain of its symmetry mate. The N-termini of the heavy and light chains are, however, arranged around one of the two remaining twofold axis each. Interestingly, both chain termini result from proteolytic cleavages that appear during proenzyme activation, whereas the head-to-tail arrangement involves chain termini, already present in the zymogen. This suggests that the head-to-tail arrangement observed in the crystal structure originates from the zymogen form, whereas the N-termini contacts are suggested to be formed during tetramer formation. The 87 residue propeptide, cleaved off during activation, not only blocks access to the active site of the enzyme, but also prevents formation of the tetramer. This is in contrast to the proenzymes of related structures (Turk et al., 1996; Cygler et al., 1996; Podobnik et al., 1997). A similar role is given to the approximately eight residue insertion from Asp 371 to Leu 378, cleavage of which breaks the single polypeptide chain of the papain-like domain region into heavy and light chains.

The positioning of the residual propart domain at the end of the active site cleft and the extended contact surface with the papain-like domain leaves no doubt as to which three domain unit form the functional monomer (FIG. 10). However, the question as to whether the domains of a functional monomer originate from the same polypeptide chain, as would be assumed, is not so clear. The disconnected termini of the head-to-tail dimer (C-termini of the residual propart domains and N-termini of heavy chains) are 45A apart and visual inspection of the structure of the cathepsin B propeptide (Podobnik et al., 1997) superimposed on the structure of DPPI provides no clear hints. Therefore, resolution of this question must await a zymogen crystal structure determination.

Papain-Like Domains Structure

The two domains of the papain-like structure are termed left—(L-) and right—(R-) domains according to their position as seen in FIG. 10C The L-domain contains several alpha-helices, the most pronounced being the structurally conserved 28 residue long central alpha-helix with catalytic Cys 234 on its N-terminus. The R-domain is a beta-barrel with a hydrophobic core. The interface of the two domains is quite hydrophobic, in contrast to the interface of the cathepsin B structure (Musil et al., 1991), which is stabilised by numerous salt bridges. The interface opens in front, forming the active site cleft, in the middle of which is the catalytic ion pair of the Cys 234 and His 381.The papain-like domains contain nine cysteines, six of them being involved in disulfide bridges (231-274, 267-307, 297-313) and three being free (catalytic Cys 234, Cys 331 and Cys 424). The side chain of Cys 424 is exposed to the solvent and is the major binding site for the osmium and the only site for the gold derivative, whereas the side chain of Cys 331 is buried into the hydrophobic environment of the side chains of Met 336, Met 346, Val 324 and Ala 430.

Residual Propart Domain Structure

The residual propart domain forms an enclosed structure allowing it to fold independently from the rest of the enzyme (Cigic et al., 2000). This domain folds as an up-and-down beta-barrel composed of eight antiparallel beta-strands wrapped around a hydrophobic core formed by tightly packed aromatic and branched hydrophobic side chains. The strands are numbered consecutively as they follow each other in the sequence. The residual propart domain contains four cysteine residues, which form two disulfide bridges (Cys 6-Cys 94, Cys 30-Cys 112). The N-terminal residues from Asp 1 to Gly 13 seal one end of the beta-barrel, whereas there is a broad groove filled with solvent molecules and a sulfate ion at the other end (FIGS. 10c, d).

Two long loops project out of the beta-barrel. The first, (Ser 24-Gln 36) is a broad loop from the beta-strand number 1, shielding the first and the last strands from solvent. This loop additionally stabilizes the barrel structure via the disulfide Cys 30-Cys 112, which fastens the loop to strand 8. The second loop (Lys 82-Tyr 93), termed hairpin loop, is a two strand beta-sheet structure with a tight beta-hairpin at its end. The loop comes out of strands 7 and 8 and encloses the structure by the disulfide Cys 6-Cys 94 which connects the loop to the N-terminus of the residual propart domain. This loop stands out of the tetrameric structure (FIGS. 10a, c) and is reminiscent of cathepsin X 110-123 loop (Guncar et al., 2000) by its pronounced form and charged side chains, indicating a possible common role of these structural features.

Interface of Papain-Like Domains and the Residual Propart Domain

All three domains make contacts along the edges of the two papain-like domains and form a large binding surface of predominantly hydrophobic character. The wall is formed by beta-strands 4 to 7 of the residual propart domain that attaches to the surface of the papain-like domains. There are three stacks of parallel side chains from each of the strands of the beta-sheet, mentioned above, interacting in a zipper-like manner with the side chains of a short three turn alpha-helix between Phe 278-Phe 290. This feature is a conserved structural element in all homologous enzymes. The middle turn of this helix contains an additional residue, Ala 283, thus forming a pi helical turn, which is a unique feature of DPPI. The branched side chain of Leu 281 is the central residue of a small hydrophobic core formed at the interface of the three domains. Only the side chain of Glu 69 escapes the usual beta-sheet side chain stacking and forms a salt bridge with Lys 285. The exchange of electrostatic interactions continues from Lys 285 towards the side chains of His 103 and Asp 289.

The Active Site Cleft

The four active site clefts are positioned approximately at the tetrahedral corners of the molecule, about 50 to 60 Å apart and are exposed to the solvent. Each active site cleft is formed by features of all three domains of a functional monomer of DPPI (FIG. 11), the papain-like domains forming the sides of the monomer which is closed at one end by the residual propart domain.

The reactive site residues Cys 234(25)-His 381(159) form an ion pair and are at their usual positions above the oxyanion hole formed by the amides of Gln 228 (19) side chain and Cys 234(25) main chain. An HE1 hydrogen atom from a ring of Trp 405(177) is in the correct orientation to bind a substrate carbonyl atom of a P1′ residue and the extended stretch of conserved Gly 276(65)-Gly 277(66) is in the usual place to bind a substrate P2 residue with an anti-parallel hydrogen bond ladder (Turk et al., 1998d). The resulting hydrogen bonds are indicated in FIG. 11. (For easier sequence comparison, the papain numbering is given in parentheses.)

As expected, the substrate binding area beyond the S2 binding site is blocked. DPPI utilizes the residual propart domain to build a wall, which prevents formation of a binding surface beyond the S2 substrate binding site. This wall spans across the active site cleft as well as away from it. A broad loop made of the N-terminal five residues surrounds the S2 binding site and forms a layer across the active site cleft. The blockade of the cleft is additionally enhanced by carbohydrate rings attached to Asn 5. (The first carbohydrate ring is well resolved by the electron density map.) Behind the N-terminal loop, there is an upright beta-hairpin (Lys 82-Tyr 93), which protrudes far into the solvent.

Substrate Binding Sites

Surprisingly, the anchor for the N-terminal amino group of a substrate is not the C-terminal carboxylic group of a peptide chain, as expected based on analogy with cathepsin H (Guncar et al., 1998) and bleomycin hydrolase (Joshua-Tor et al., 1995), but instead, it is the carboxylic group of the Asp 1 side chain, the N-terminal residue of the residual propart domain (FIG. 11). The N-terminal amino group of Asp 1 is fixed with two hydrogen bonds between the main chain carbonyl of Glu 275 and the side chain carbonyl of Gln 272. The Asp 1 side chain reaches towards the entrance of the S2 binding site, where it interacts with the electrostatically positive edge of the Phe 278 ring (FIG. 11).

The side chains of Ile 429, Pro 279, Tyr 323 and Phe 278 form the surface of the S2 binding site. This site has a shape of a pocket, and is the deepest such known this far. The bottom of the pocket is filled with an ion and two solvent molecules. The high electron density peak, chemical composition of the coordinated atoms, and the requirement of DPPI for chloride ions, lead to the conclusion that this ion is chloride. It is positioned at the N-terminal end of the three-turn helix (Phe 278-Phe 290) and is coordinated by the main chain amide group of Tyr 280 (3.2 Å and 3.3 Å) away from hydroxyl group of Tyr 323 and two solvent molecules (FIG. 11). The ring of Phe 278 is thus positioned with its electro-positive edge between the negative charges of chloride and Asp 1 carboxylic group.

The surfaces of the other substrate binding sites (S1, S1′, S2′) show no features unique for DPPI, when compared with other members of the family (Turk et al., 1998d). The S1 binding site is placed between the active site loops Gln 272-Gly 277 and Gln 228-Cys 234, beneath the disulfide 274-231 and Glu 275. The S1′ substrate binding site is rather shallow with a hydrophobic surface contributed by Val 352 and Leu 357 and the S2′ binding site surface is placed within the Gln 228-Cys 234 loop. The molecular surface along the active site cleft beyond the S2′ binding area is wide open, indicating that there is no particular site defined for binding of substrate residues.

Mechanisms of Exopeptidases: Peptide Patches and the Residual Propart Domain

Elucidation of the structure of DDPI explains its unique exopeptidase activity. FIG. 12 clearly shows that converting endo- to exo-peptidase activity of a papain-like protease is achieved by features added on either side of the active site cleft to the structure of a typical papain-like endo-peptidase framework (Turk et al., 1998d; McGrath, 1999). Carboxypeptidases cathepsins B (Musil et al., 1991) and X (Guncar et al., 2000) utilise loops which block access along the primed side and provide histidine residues to anchor the C-terminal carboxylic group of a substrate. In contrast, the amino peptidases cathepsin H (Guncar et al., 1998) and a more distant homolog bleomycin hydrolase (Joshua-Tor et al., 1995) utilise a polypeptide chain in an extended conformation that blocks access along the non-primed binding sites and provides its C-terminal carboxylic group as the anchor for the N-terminal amino group of a substrate. DPPI recognizes the N-terminal amino group of a substrate in a unique way. The anchor is a charged side-chain group of the N-terminal residue Asp 1, folded as a broad loop on the surface. However, this loop is not a part of a polypeptide chain of the papain-like domains, but belongs to an additional domain. It has an independent origin that adds to the framework of a papain-like endopeptidase and turns it into an exopeptidase. The residual propart domain excludes any endopeptidase activity of the enzyme.

Substrate Excluding Specificity of DPPI

The selectivity of DPPI is best described by exclusion rules and the disclosed structure provides a variety of clues for understanding their mechanism.

DPPI shows no endopeptidase activity in contrast to cathepsins B and H. It is, however, inhibited by cystatin type inhibitors, non-selective protein inhibitors of papain-like cysteine proteases (Turk et al., 2000), as are the other papain-like exopeptidases, i.e. cathepsins B, H, and X. The patches on the papain-like endopeptidase structure framework responsible for cathepsins B and H exopeptidase activity are relatively short polypeptide fragments, which lie on the surface (Musil et al., 1991; Guncar et al., 1998). It was shown for the cathepsin B occluding loop (Illy et al., 1997; Podobnik et al., 1997) that these rather flexible structural features compete with substrates and inhibitors for the same binding sites within the active site cleft. A similar function has been suggested for the cathepsin H mini-chain (Guncar et al., 1998). Analogously, the flexibility of the five N-terminal residues of the residual propart domain can explain the complex formation of DPPI with cystatin type inhibitors. However, proximal to this short region is the massive body of the residual propart domain with its extended binding surface for the papain-like domain and its projecting feature beta-hairpin Lys 82-Tyr 93 tightly fastened within the tetrameric structure. Therefore, it is highly unlikely that the residual propart domain could be pushed away by an approaching polypeptide. This indicates the robust mechanism by which endopeptidase activity of DPPI is excluded. Control on the micro level is then achieved by the carboxylate group of the Asp 1 side chain, which is oriented towards the active site cleft to rule out approach of substrate without an N-terminal amino group (McGuire et al., 1992), as demonstrated in FIG. 11.

DPPI, similarly to most other papain-like proteases, does not cleave substrates with proline at P1 or P1′ position. A simple modeling study suggests that proline residues at these positions would disturb the hydrogen bonding network and may produce clashes in the S1 substrate binding site.

The side chain carboxylate group points towards the S2 substrate binding site, where it can bind to the N-terminal NH3+group of the substrate, thereby directing dipeptidyl aminopeptidase specificity. Positive charges on lysine and arginine residues could interact with Asp1 resulting in a re-positioning of the substrate and explain why substrates with these side chains at the N-terminal are not cleaved.

The Residual Propart Domain is a Structural Homolog of a Protease Inhibitor

For the residual propart domain, no sequence homolog is known, however, 44 similar structural folds were found using DALI (Holm and Sander, 1996). The highest similarity scores were obtained with the structures of streptavidin (1SWU) and erwinia chrysanthemi inhibitor (1SMP), whose structure was determined in complex with the serratia metallo-protease (Baumann et al., 1995). (The codes in parentheses are Protein Data Bank accession numbers.)

The large number of structural homologs is not surprising, as the eight-stranded antiparallel beta-barrels are a common folding pattern. However, the geometry of binding the erwinia chrysanthemi inhibitor to metallo-protease also points to a functional similarity. The N-terminal tail of erwinia chrysanthemi inhibitor binds into the active site cleft of the serratia marcescens metallo-protease along the substrate binding sites towards the active site cleft. Even the chain traces of the N-terminal parts are similar, i.e., an extended chain, which continues into a short helical region (FIG. 13). In contrast to the residual propart domain of DPPI, which enters the active site cleft from the non-primed region (in a substrate-like direction), the N-terminal tail of erwinia chrysanthemi inhibitor binds along the primed substrate binding sites (in the direction opposite to that of a substrate). It is thus intriguing to suggest that the residual propart domain is an adapted inhibitor, which does not abolish the catalytic activity of the enzyme, but prevents its endopeptidase activity by blocking access to only a portion of the active site cleft.

Genetic Disorders Located on DPPI Structure

Quite a few of the genetic disorders of DPPI described are nonsense mutations resulting in truncation of the expressed sequence (Hart et al., 1999; Toomes et al., 1999). However, there is a series of missense mutations (D212Y, V225F, Q228L, R248P, Q262R, C267Y, G277S, R315c and Y323C) in the sequence of the heavy chain (FIG. 6a) (Toomes et al., 1999; Hart et al., 2000a; Hart et al., 2000b; Allende et al., 2001). Their structure based interpretation suggests that not all missense mutations necessarily result in complete loss of DPPI activity.

Gln 228 and Gly 277 are two of the key residues involved in substrate binding. Mutation of Q228L disrupts the oxyanion hole surface and consequently severely effects productive binding of the carbonyl oxygen of the scissile bond of the substrate. The G277S mutation presumably disrupts the main chain—main chain interactions with the P2 residue, as the glycine conformation can not be preserved (see FIG. 11).

The most frequent missense mutation appears to be the Y323C (Toomes et al., 1999; Hart et al., 2000b). Normally the hydroxyl group of Tyr 323 is involved in the binding of the chloride ion, which seems to stabilize the S2 substrate binding site (FIG. 14b). The mutation into a cysteine may not only disrupt chloride binding but also positioning of the Phe 278 and consequently Asp 1. The change to a cysteine residue carries yet more impact. It may alter the structure of the short segment of the chain towards Cys 331 by forming a new disulfide bond. Even the binding surface for the residual propart domain may be disrupted and it is possible that this mutant may not form an oligomeric structure at all and may thus even exhibit endopeptidase activity.

The mutations C267Y, R315c and Q262R are located around the surface loop enclosed by the disulfide Cys 297-Cys 313. In the observed structure, the side chains of Gln 262 and Phe 298 form the center around which the loop is folded (FIG. 14a). Cys 267 is located in the vicinity of Gln 262 and fastens the structure of the loop via the disulfide Cys 267-Cys 307. Arg 315 is involved in a salt bridge with Glu 263, the residue following the central loop residue Gln 262, and is adjacent to Cys 313. Either of these mutations may thus prevent proper folding of the loop and disrupt formation of the two disulfides. Free cysteines may thus result in non-native disulfide connectivity, which has the potential to aggregate the improperly folded DPPI monomers.

The R248P mutant presumably leads to folding problems as a proline at this position quite likely breaks the central helix at the second turn from its C-terminus. A phenylalanine ring at the position of Val 225 is too large to form the basis of the short loop Asn 403-Gly 413 and thereby disrupts the primed substrate binding sites, in particular the positioning of the conserved Trp 405 involved in P1′ residue binding (see FIG. 11).

The mutation D212Y, however, seems to represent a special case. It does not appear to be linked to the active site structure or aggregation problems. Asp 212, the 6th residue from the N-terminus of the papain-like domain, is exposed to the surface where it forms a salt bridge with Arg 214. Disruption of the salt bridge structure may result in a different positioning of the N-terminus and since the N-terminal region is involved in molecular symmetry contacts, this mutation may prevent tetramer formation (FIG. 14c).

DPPI is a Protease Processing Machine

Oligomeric proteolytic machineries as 20S proteasome (Lowe et al., 1995; Groll et al., 1997), bleomycin hydrolase (Joshua-Tor et al., 1995), or tryptase (Pereira et al., 1998) restrict access of substrates to their active sites. Proteasomes are barrel-like structures composed of four rings of alpha and beta-subunits, which cleave unfolded proteins captured in the central cavity into short peptides. Tryptases are flat tetramers with a central pore in which the active sites reside. The pore restricts the size of accessible substrates and inhibitors. And also the active sites of bleomycin hydrolase are located within the hexameric barrel cavity. In contrast, the active sites of DPPI are located on the external surface, allowing the tetrahedral architecture to introduce a long distance between them, which allows them to behave independently. This turns DPPI into a protease capable of hydrolysis of protein substrates in their native state, regardless of their size. It's robust design, supported by the oligomeric structure, confines the activity of the enzyme to an aminodipeptidase and thereby makes it suitable for use in many different environments, where DPPI can selectively activate quite a large group of chymotrypsin-like proteases.

Protein Purification and Crystallization

DPPI was expressed in the insect cell/bacullovirus system as described above. The purified DPPI was concentrated to 10 mg/ml in a spin concentrator (CENTRICON®, AMICON®). Crystals were grown using sitting drop vapor diffusion method. The reservoir contained 1 ml of 2.0 M ammonium sulphate solution with 0.1M sodium citrate and 0.2M potassium/sodium tartrate at pH 5.6 (Hampton screen II, solution 14). The drop was composed of 2 μl reservoir solution and 2 μl of protein solution. Acetic acid and Na-hydroxide were used to adjust pH.

The crystals of DPPI belong to the orthorhombic space group 1222 with cell dimensions a=87.15 Å, b=88.03 Å, and c=114.61 Å. Native crystals diffracted to 2.15 Å resolution on XRD1 beamline in Elettra. Before data collections, crystals of DPPI were soaked in 30% glycerol solution before they were dipped into liquid nitrogen and frozen. All data sets were processed using the program DENZO® (Otwinowski and Minor, 1997).

Phasing and Structure Solution

The position of the enzymatic domain was determined by molecular replacement implemented in the EPMR program (Kissinger et al., 1999) using various cathepsin structures. The partial model did not enable the inventors to proceed with the structure determination, therefore a heavy atom derivative screen was performed. Two soaks proved successful (K₂Cl₆Os₃ and AuCl₃). A three wavelength MAD data set of osmium derivative was measured at Max-Planck beamline at DESY Hamburg. Native data set had to be used as a reference to solve the heavy atom positions and treat the MAD data as MIR data. The RSPS program (Knight, 1989) suggested a single heavy atom position. The derived map was not of sufficient quality to enable model building. It did, however, show that the molecular replacement solution and MAD/MIR map were consistent. Phasing based on a single gold heavy atom site and an additional five minor osmium heavy atom sites located from the residual maps, refined and solvent flattened with SHARP (de La Fortelle and Bricogne, 1997) using data to 3.0 Å, resulted in an interpretable electrone density map.

Refinement and Structure Validation

This structure was then refined to an R-value of 0.184 (R-free 23.8 using 5% of reflections) against 2.15 Å resolution data. When using 2.6 Å data, individual B-value refinement was included and with 2.4 Å resolution data and R-value about 0.24, the inclusion of solvent molecules was initiated using an automated procedure. The chloride ion was identified from a water molecule, which, after positional and B-value refinement, returned a B-value for oxygen at the minimum boundary. It was still positioned within a 4.5 sigma positive peak of the Fo-Fc difference electron density map. Three sulfate ions were found by visual inspection of large clouds of positive density, contoured at 3.0 sigma in the vicinity of already built solvent molecules. The only carbohydrate ring observed was attached to Asn 5 in the residual propart domain. It was recognized from a cluster of solvent molecules and peaks of positive density in Fo-Fc map and positioned among them.

All model building steps, structure refinement and map calculations were done using MAIN (Turk, 1992) running on COMPAQ® Alpha workstations. The Engh and Huber force field parameter set was used (Engh and Huber, 1991). Structure analysis was performed with MAIN during the entire course of model building and refinement: particularly useful were averaged kicked-maps which, in the cases of doubt, pointed to the correct electron density interpretation. The final model was inspected and validated with the program WHAT CHECK (Hooft et al., 1996).

The substrate model using the N-terminal sequence of granzyme A ERIIGG, was generated on the basis of crystal structures of papain family enzymes complexed with substrate mimicking inhibitors, as described (Turk et al., 1995). Binding of substrate residues P2 and P1 into the S2 and S1 binding sites was indicated by chloromethylketone substrate analogue inhibitors bound to papain (Drenth et al., 1976). The binding of P1′ and P2′ residues into the S1′ and S2′ binding sites was suggested by CA030 in complex with cathepsin B (Turk et al., 1995). The model was built manually on superimposed structures and then energetically minimized under additional distance constraints that preserved the consensus hydrogen bonding network between the substrate and underlying enzymatic surface. The binding geometry of the P3′ and P4′ residues was generated in an extended conformation and minimized with no additional distance restraints.

TABLE 4
Diffraction data and refinem nt statistics

| Data set (wavelength) | Nat. 1.0 Å | Os 1.13987 Å | Os 1.139205 Å | Os 1.04591 | Au |
|---|---|---|---|---|---|
| Spacegroup | I222 | | | | |
| Cell axis (a, b, c) | a = 87.154 | | | | |
| | b = 88.031 | | | | |
| | c = 114.609 | | | | |
| Resolution range | 20-2.15 | 20-2.81 | 20-2.82 | 20-2.68 | 20-3.0 |
| Total measurements | 96833 | 71728 | 80430 | 79013 | 11889 |
| Unique reflections | 23553 | 18594 | 19651 | 21720 | 3511 |
| Completeness (last | 0.976(0.99) | 0.90(0.70) | 0.95(0.76) | 0.81(0.76) | 0.78 |
| shell) | | | | | |
| Anom. Comp. | | 0.75 | 0.84 | 0.75 | |
| R-sym. | 0.070(0.249) | 0.055(0.184) | 0.063(0.175) | 0.056(0.483) | 0.053(0.109) |
| Phas. isom. acnt (cntr) | | 0.57(0.38) | 0.59(0.39) | 0.64(0.52) | 0.52 |
| Pow. anom. acnt | | 0.23 | 0.31 | 0.23 | |
| FOM acnt (cntr) | 0.51(0.24) | | | | |
| Protein atoms | 2749 | | | | |
| Solvent | 467 | | | | |
| Sulphate ions | 3 | | | | |
| Chloride ion | 1 | | | | |
| Resolution in refinement | 10.0-2.15 | | | | |
| Reflections in | 23353 | | | | |
| refinement | | | | | |
| R-factor | 0.186 | | | | |
| R-free | | | | | |
| Average B | 24.8 | | | | |
| Bond rms deviations | 0.0090 | | | | |
| Angle rms deviations | 1.62 | | | | |

### LISTING OF REFERENCES

1. Allende, L. M., Garcia-Perez, M. A., Moreno, A., Corell, A., Corasol, M., Martinez-Canut, P. and Arnaiz-Villena, A. (2001). Cathepsin C gene: First compound heterozygous patient with Papillon-Lefevre syndrome and novel symptomless mutation. Hum. Mutat. 17, 152-153.
2. Baumann, U., Bauer, M., Letoffe, S., Delepelaire, P., Wandersman, C (1995). Crystal structure of a complex between Serratia marcescens metallo-protease and an inhibitor from Erwinia chrysanthemi. J. Mol. Biol. 248, 653-661.
3. Blundell, T. L., Johnson, N. L. (1976) Protein Crystallography, Academic Press.
4. Brunger, A. T., Adams, P. D., Clore, G. M., DeLano, W. L., Gros, P., Grosse-Kunstleve, R. W., Jiang, J. S., Kuszewski, J., Nilges, M., Pannu, N. S., Read, R. J., Rice, L. M., Simonson, T., Warren, G. L. (1998) Acta Crystallogr. D 54, 905-921.
5. Carson, M. (1991). Ribbons 2. J. Appl. Cryst. 24, 283-291.
6. Cigic, B., Dahl, S. W. and Pain, R. H. (2000). The residual pro-part of cathepsin C fulfills the criteria required for an intramolecular chaperone in folding and stabilizing the human proenzyme. Biochemistry 39, 12382-90.
7. Cigic, B., Krizaj I., Kralj, B., Turk, V. and Pain, R. H. (1998). Stoichiometry and heterogeneity of the pro-region chain in tetrameric human cathepsin C Biochim. Biophys. Acta. 1382, 143-50.
8. Cowtan, K., Main, P. (1998) Acta Crystallogr. D 54, 487-493.
9. Cygler, M., Sivaraman, J., Grochulski, P., Coulombe, R., Storer, A. C and Mort, J. (1996). Structure of rat procathepsin B: model for inhibition of cysteine protease activity by the proregion. Structure 4, 405-416.
10. Dahl, S. W., Halkier T., Lauritzen, C, Dolenc, I., Pedersen, J., Turk, V. and Turk, B. (2001). Human recombinant pro-dipeptydil peptidase I (cathepsin C) can be activated by cathepsins L and S but not by autocatalytic processing. Biochemistry 40, 1671-1678.
11. Darmon, A. J., Nicholson, D. W., Bleackley, R. C (1995). Activation of the apoptotic protease CPP32 by cytotoxic T-cell-derived granzyme B. Nature 377, 446-8.
12. de La Fortelle, E. and Bricogne, G. (1997). Methods in Enzymology, Macromolecular Crystallography, 276, 472-494.
13. Dolenc, I., Turk B., Pungercic, G., Ritonja, A. and Turk, V. (1995). Oligomeric structure and substrate induced inhibition of human cathepsin C J. Biol. Chem. 270, 21626-31.
14. Dolenc, I., Turk, B., Kos, J. and Turk, V. (1996). Interaction of human cathepsin C with chicken cystatin. FEBS Lett. 392, 277-80.
15. Doling et al. (1996) FEBS Lett. 392, 277-280.
16. Drenth, J., Kalk, K. H. and Swen, H. M. (1976). Binding chloromethyl ketone substrate analogues to crystalline papain. Biochemistry 15, 3731-3738.
17. Engh, R. A. and Huber, R. (1991). Accurate bond and angle parameters for X-ray protein structure refinement. Acta. Cryst. A47, 392-400.
18. Fruton, J. S, and Mycek, M. J. (1956). Studies of beef spleen cathepsin C Arch. Biochem. Biophys. 65, 11-20.
19. Garman, E. (1999) Acta Crystallogr. D 55, 1641-1653.
20. Groll, M., Ditzel, L., Lowe, J., Stock, D., Bochtler, M., Bartunik, H. D. and Huber, R. (1997). Structure of 20S proteasome from yeast at 2.4 Å resolution. Nature 386, 463-71.
21. Gruenwald et al. (1993) Procedures and Methods Manual, 2nd ed., Pharmigen, San Diego, Calif. p. 44-49.
22. Gruenwald et al. (1993) Procedures and Methods Manual, 2nd ed., Pharmigen, San Diego, Calif. p. 52-53.
23. Guncar, G., Klemenicic, I., Turk, B., Turk, V., Karaoglanovic-Carmona, A., Juliano, L. and Turk D. (2000). Crystal structure of cathepsin X: a flip-flop of the ring of His23 allows carboxy-monopeptidase and carboxy-dipeptidase activity of the protease. Structure 29, 8:305-313.
24. Guncar, G. et al. (1998). Crystal structure of porcine cathepsin H determined at 2.1 Å resolution: location of the mini-chain Crystal structure of porcine cathepsin H determined at 2.1 Å resolution: location of the mini-chain C-terminal carboxyl group defines cathepsin H aminopeptidase function. Structure 6(1):51-61.
25. Gutman, H. R. and Fruton, J. (1948). On the proteolytic enzymes of animla tissues VIII: An Intracellular enzyme related to chymotrypsin. J. Biol. Chem. 174, 851-858.
26. Hart, T. C, Hart, P. S., Bowden, D. W., Michalec, M. D., Callison, S. A., Walker, S. J., Zhang, Y. and Firatli, E. (1999). Mutations of the cathepsin C gene are responsible for Papillon-Lefevre syndrome. J. Med. Genet. 36, 881-887.
27. Hart, T. C, Hart, P. S., Michalec, M. D., Zhang, Y., Firatli, E., Van Dyke, T. E., Stabholz, A., Zlorogorski, A., Shapira, L. and Soskolne, W. A. (2000a). Haim-Munk syndrome and Papillon-Lefevre syndrome are allelic mutations in cathepsin C J. Med. Genet. 37, 88-94.
28. Hart, T. C, Hart, P. S., Michalec, M. D., Zhang, Y., Marazita, M. L., Cooper, M., Yassin, O. M., Nusier, M. and Walker, S. (2000b). Localisation of a gene for prepubertal periodontitis to chromosome 11q14 and identification of a cathepsin C gene mutation. J. Med. Genet. 37, 95-101.
29. Holm, L. and Sander, C (1996). Mapping the protein universe. Science 273, 595-602.
30. Hooft, R. W. W. Vriend, G. Sander, C Abola, E. E. (1996). Errors in protein structures. Nature 381, 272-272.
31. Illy, C, Quraishi, O., Wang, J., Purisima, E., Vernet, T., Mort, J. S. (1997). Role of the occluding loop in cathepsin B activity. J. Biol. Chem. 272, 1197-202.
32. Ishidoh et al. J. Biol. Chem. (1991) 266, 16312-16317.
33. Joshua-Tor, L., Xu H. E., Johnston, S. A. and Rees, D. C. (1995). Crystal structure of a conserved protease that binds DNA: the bleomycin hydrolase, Gal6. Science 269, 945-50.
34. Kissinger, C R., Gehlhaar, D. K. and Fogel, D. B. (1999). Rapid automated molecular replacement by evolutionary search. Acta Cryst. D Biol. Crystallogr. 55, 484-491.
35. Knight, S. (1989). “Ribulose 1,5-Bisphosphate Carboxylase/Oxygenase—A Structural Study”. Thesis, Swedish University of Agricultural Sciences, Uppsala.
36. Kumar, S. (1999). Mechanisms mediating caspase activation in cell death. Cell Death Diff. 6, 1060-6.
37. Laskowski et al. (1993) J. Appl. Cryst. 26, 283-291.
38. Lauritzen et al. (1998) Protein Expr. Purif. 14, 434-442.
39. Lowe, J., Stock, D., Jap, B., Zwickl, P., Baumeister, W. and Huber, R. (1995). Crystal structure of the 20S proteasome from the archaeon T. acidophilum at 3.4 Å resolution. Science 268, 533-9.
40. Luthy et al. (1992) Nature 356, 83-85.
41. Lynch, G. W. and Pfueller, S. L. (1988). Thrombin-independent activation of platelet factor XIII by endogenous platelet acid protease. Thromb. Haemost. 59, 372-7.
42. McDonald, J. K., Reilly, T. J., Zeitman, B. B. and Ellis, S. (1966). Cathepsin C: a chloride-requiring enzyme. Biochem. Biophys. Res. Commun. 8, 771-775.
43. McGrath, M. E. (1999). The Lysosomal Cysteine Proteases. Annu. Rev. Biophys. Biomol. Struct. 28, 1818-204.
44. McGuire, M. J., Lipsky, P. E. and Thiele, D. L. (1992). Purification and characterization of dipeptidyl peptidase I from human spleen. Arch. Biochem. Biophys. 295, 280-8.
45. Merritt, E. A. and Bacon, D. J. (1997). Raster3D: Photorealistic Molecular Graphics. Methods in Enzymology, 277, 505-524.
46. Metrione, R. M. et al (1966). Biochemistry 5, 1597-1604.
47. McDonnald J. K. et al (1969). J. Biol. Chem. 244, 2693-2709.
48. Muno, D., Ishidoh, K., Ueno, T. and Kominami, E. (1993). Processing and transport of the precursor of cathepsin C during its transfer into lysosomes. Arch. Biochem. Biophys. 306, 103-10.
49. Musil, D. Zucic, D., Turk, D., Engh, R. A., Mayr, I., Huber, R., Popovic, T., Turk, V., Towatari, T., Katunuma, N., Bode, W. (1991). The refined 2.15A X-ray crystal structure of human liver cathepsin B: the structural basis for its specificity. EMBO Journal, 10, 2321-2330.
50. Nauland, U. and Rijken, D. C (1994). Activation of thrombin-inactivated single-chain urokinase-type plasminogen activator by dipeptidyl peptidase I (cathepsin C). Eur. J. Biochem. 223, 497-501.
51. Navaza, J. (1993) Acta Crystallogr. D 49, 588-591.
52. Navaza, J. (1994) Acta Crystallogr. A 50, 157-163.
53. Navaza, J., Vernoslova, E. (1995) Acta Crystallogr. A 51, 445-449.
54. Nelson, R. M. and Long, G. L. (1989) A general method of site-specific mutagenesis using a modification of the Thermus aquaticus polymerase chain reaction. Anal. Biochem. 180, 147-51.
55. Neurath, H. (1984). Evolution of proteolytic enzymes. Science 224, 350-357.
56. Nicholls, A., Sharp, K. A. and Honig, B. (1991). Protein folding and association: insights from the interfacial and thermodynamic properties of hydrocarbons. Proteins 11, 281-376.
57. Nuckolls, G. H. and Slavkin, H. C (1999). Paths of glorious proteases. Nat. Genet. 23, 378-80.
58. Otwinowski, Z. and Minor, V. (1997). Processing of X-ray diffraction data collection in osciallation mode. Methods in Enzymology, Macromolecular Crystallography, 276, 307-326.
59. Paris, A., Strukelj, B., Pungercar, J., Renko, M., Dolenc, I. and Turk, V. (1995). Molecular cloning and sequence analysis of human preprocathepsin C FEBS Lett. 369, 326-30.
60. Pereira, P. J., Bergner A., Macedo-Ribeiro, S., Huber, R., Matschiner, G., Fritz, H., Sommerhoff C P. and Bode W. (1998). Human beta-tryptase is a ring-like tetramer with active sites facing a central pore. Nature 392, 306-11.
61. Pham, C T. and Ley, T. J. (1999). Dipeptidyl peptidase I is required for the processing and activation of granzymes A and B in vivo. ProC Natl. Acad. Sci. USA 96, 8627-32.
62. Planta, R. J., Gorter, J. and Gruber, M. (1964). The catalytic properties of cathepsin C Biochim. Biophys. Acta 89}, 511-519.
63. Podack, E. R. (1999). How to induce involuntary suicide: The need for dipeptidyl peptidase I. ProC Natl. Acad. Sci. USA 96, 8312-8314.
64. Podobnik, M., Kuhelj, R., Turk, V. and Turk, D. (1997). Crystal structure of the Wild-type Human Procathepsin B at 2.5.backslash.AA Resolution Reveals the Native Active Site of a Papain-like Cysteine Protease Zymogen. J. Mol. Biol. 271, 774-788.
65. Rodriguez et al. (1998).
66. Rowan, A. D., Mason, P., Mach L. and Mort, J. S. (1992). Rat procathepsin B. Proteolytic processing to the mature form in vitro. J. Biol. Chem. 267, 15993-9.
67. Shresta, S., Graubert, T. A., Thomas, D. A., Raptis, S. Z. and Ley T. J. (1999). Granzyme A initiates an alternative pathway for granule-mediated apoptosis. Immunity 10, 595-605.
68. Shresta, S., Pham, C T., Thomas, D. A., Graubert, T. A. and Ley T. J. (1998). How do cytotoxic lymphocytes kill their targets. Curr. Opin. Immunol. 10, 581-7.
69. Thompson et al. (1994) Nucleic Acids Res. 22, 4673-4680.
70. Toomes, C, James, J., Wood, A. J., Wu, C L., McCormick, D., Lench, N., Hewitt, C, Moynihan, L., Roberts, E., Woods, C G., Markham, A., Wong, M., Widmer, R., Ghaffar, K. A., Pemberton, M., Hussein, I. R., Temtamy, S. A., Davies, R., Read, A. P., Sloan, P, Dixon, M. J. and Thakker NS. (1999). Loss-of-function mutations in the cathepsin C gene result in periodontal disease and palmoplantar keratosis. Nat. Genet. 23, 421-4.
71. Travis, J. (1988). Structure, function, and control of neutrophil proteinases. Am. J. Med. 84, 37-42.
72. Turk D.: Proceedings from the 1996 meeting of the International Union of Crystallography Macromolecular Macromolecular Computing School, eds P. E. Bourne & K. Watenpaugh.
73. Turk, B. Dolenc, I. and Turk, V. (1998b). 214 Dipeptidyl-peptidase I. Handbook of proteolytic enzymes. (Barrett, A. J., Rawlings, N. D., Woessner, J. F. Jr., eds.) Academic Press Ltd., London, 631-634.
74. Turk, B., Turk, D. and Turk, V. (2000). Lysosomal cysteine proteases: more than scavengers. Biochim. Biophys. Acta. 1477, 98-111.
75. Turk, D. (1992). Weiterentwicklung eines Programms fur Molekulgraphik und Elektrondichte-Manipulation und seine Anwendung auf verschiedene Protein-Strukturauflclarungen. Ph. Thesis, Technische Universitat, Munchen.
76. Turk, D., Guncar, G., Podobnik, M., and Turk, B. (1998d). Revised definition of substrate binding sites of papain-like cysteine proteases. Biol. Chem. 379, 137-147.
77. Turk, D., Podobnik, M., Kuhelj, R. Dolinar, M. and Turk, V. (1996). Crystal structures of human procathepsin B at 3.2 and 3.3 Å resolution reveal an interaction motif between a papain like cysteine protease and its propeptide. FEBS Lett. 384, 211-214.
78. Turk, D., Podobnik, M., Popovic, T., Katunuma, N., Bode, W., Huber, R. and Turk, V. (1995). Crystal Structure of Cathepsin B inhibited with CA030 at 2.backslash.AA Resolution: A basis for the Design of Specific Epoxysuccinyl Inhibitors. Biochemistry 34, 4791-4797.
79. Wolters, P. J., Laig-Webster, M. and Caughey, G. H. (2000). Dipeptidyl peptidase I cleaves matrix-associated proteins and is expressed mainly by mast cells in normal dog airways. Am. J. Respir. Cell Mol. Biol. 22, 183-90.
80. Wolters, P. J., Pham, C T. N., Muilenburg, D. J., Ley, T. J. and Caughey, G. H. (2001). Dipeptidyl Peptidase I is Essential for Activation of Mast Cell Chymases, but not Tryptases, in Mice. J. Biol. Chem., in press.

| Sequence CWU 1 | |
|---|---|
| rattus norvegicus | |
| Met Gly Pro Trp Thr His Ser Leu Arg Ala Ala Leu Leu Leu Val Leu Leu Gly Val | |
| Cys Thr Val Ser Ser Asp Thr Pro Ala Asn Cys Thr Tyr Pro Asp Leu Leu Gly Thr | |
| Trp Val Phe Gln Val Gly Pro Arg His Pro Arg Ser His Ile Asn Cys Ser Val Met | |
| Glu Pro Thr Glu Glu Lys Val Val Ile His Leu Lys Lys Leu Asp Thr Ala Tyr Asp | |
| Glu Val Gly Asn Ser Gly Tyr Phe Thr Leu Ile Tyr Asn Gln Gly Phe Glu Ile Val | |
| Leu Asn Asp Tyr Lys Trp Phe Ala Phe Phe Lys Tyr Glu Val Lys Gly Ser Arg Ala | |
| Ile Ser Tyr Cys His Glu Thr Met Thr Gly Trp Val His Asp Val Leu Gly Arg Asn | |
| Trp Ala Cys Phe Val Gly Lys Lys Met Ala Asn His Ser Glu Lys Val Tyr Val Asn | |
| Val Ala His Leu Gly Gly Leu Gln Glu Lys Tyr Ser Glu Arg Leu Tyr Ser His Asn | |
| His Asn Phe Val Lys Ala Ile Asn Ser Val Gln Lys Ser Trp Thr Ala Thr Thr Tyr | |
| Glu Glu Tyr Glu Lys Leu Ser Ile Arg Asp Leu Ile Arg Arg Ser Gly His Ser Gly | |
| Arg Ile Leu Arg Pro Lys Pro Ala Pro Ile Thr Asp Glu Ile Gln Gln Gln Ile Leu | |
| Ser Leu Pro Glu Ser Trp Asp Trp Arg Asn Val Arg Gly Ile Asn Phe Val Ser Pro | |
| Val Arg Asn Gln Glu Ser Cys Gly Ser 245 250 255 Cys Tyr Ser Phe Ala Ser Leu | |
| Gly Met Leu Glu Ala Arg Ile Arg Ile Leu Thr Asn Asn Ser Gln Thr Pro Ile Leu | |
| Ser Pro Gln Glu Val Val Ser Cys Ser Pro Tyr Ala Gln Gly Cys Asp Gly Gly Phe | |
| Pro Tyr Leu Ile Ala Gly Lys Tyr Ala Gln Asp Phe Gly Val Val Glu Glu Asn Cys | |
| Phe Pro Tyr Thr Ala Thr Asp Ala Pro Cys Lys Pro Lys Glu Asn Cys Leu Arg Tyr | |
| Tyr Ser Ser Glu Tyr Tyr Tyr Val Gly Gly Phe Tyr Gly Gly Cys Asn Glu Ala Leu | |
| Met Lys Leu Glu Leu Val Lys His Gly Pro Met Ala Val Ala Phe Glu Val His Asp | |
| Asp Phe Leu His Tyr His Ser Gly Ile Tyr His His Thr Gly Leu Ser Asp Pro Phe | |
| Asn Pro Phe Glu Leu Thr Asn His Ala Val Leu Leu Val Gly Tyr Gly Lys Asp Pro | |
| Val Thr Gly Leu Asp Tyr Trp Ile Val Lys Asn Ser Trp Gly Ser Gln Trp Gly Glu | |
| Ser Gly Tyr Phe Arg Ile Arg Arg Gly Thr Asp Glu Cys Ala Ile Glu Ser Ile Ala | |
| Met Ala Ala Ile Pro Ile Pro Lys Leu | |
| DNA rattus norvegicus | |
| gaattccggt tctagttgtt gttttctctg ccatctgctc tccgggcgcc gtcaaccatg | 60 |
| ggtccgtgga cccactcctt gcgcgccgcc ctgctgctgg tgcttttggg agtctgcacc | 120 |
| gtgagctccg acactcctgc caactgcact taccctgacc tgctgggtac ctgggttttc | 180 |
| caggtgggcc ctagacatcc ccgaagtcac attaactgct cggtaatgga accaacagaa | 240 |
| gaaaaggtag tgatacacct gaagaagttg gatactgcct atgatgaagt gggcaattct | 300 |
| gggtatttca ccctcattta caaccaaggc tttgagattg tgttgaatga ctacaagtgg | 360 |
| tttgcgtttt tcaagtatga agtcaaaggc agcagagcca tcagttactg ccatgagacc | 420 |
| atgacagggt gggtccatga tgtcctgggc cggaactggg cttgctttgt tggcaagaag | 480 |
| atggcaaatc actctgagaa ggtttatgtg aatgtggcac accttggagg tctccaggaa | 540 |
| aaatattctg aaaggctcta cagtcacaac cacaactttg tgaaggccat caattctgtt | 600 |
| cagaagtctt ggactgcaac cacctatgaa gaatatgaga aactgagcat acgagatttg | 660 |
| ataaggagaa gtggccacag cggaaggatc ctaaggccca aacctgcccc gataactgat | 720 |
| gaaatacagc aacaaatttt aagtttgcca gaatcttggg actggagaaa cgtccgtggc | 780 |
| atcaattttg ttagccctgt tcgaaaccaa gaatcttgtg gaagctgcta ctcatttgcc | 840 |
| tctctgggta tgctagaagc aagaattcgt atattaacca acaattctca gaccccaatc | 900 |
| ctgagtcctc aggaggttgt atcttgtagc ccgtatgccc aaggttgtga tggtggattc | 960 |
| ccatacctca ttgcaggaaa gtatgcccaa gattttgggg tggtggaaga aaactgcttt | 1020 |
| ccctacacag ccacagatgc tccatgcaaa ccaaaggaaa actgcctccg ttactattct | 1080 |
| tctgagtact actatgtggg tggtttctat ggtggctgca atgaagccct gatgaagctt | 1140 |
| gagctggtca aacacggacc catggcagtt gcctttgaag tccacgatga cttcctgcac | 1200 |
| taccacagtg ggatctacca ccacactgga ctgagcgacc ctttcaaccc ctttgagctg | 1260 |
| accaatcatg ctgttctgct tgtgggctat ggaaaagatc cagtcactgg gttagactac | 1320 |
| tggattgtca agaacagctg gggctctcaa tggggtgaga gtggctactt ccggatccgc | 1380 |
| agaggaactg atgaatgtgc aattgagagt atagccatgg cagccatacc gattcctaaa | 1440 |
| ttgtaggacc tagctcccag tgtcccatac agctttttat tattcacagg gtgatttagt | 1500 |
| cacaggctgg agacttttac aaagcaatat cagaagctta ccactaggta cccttaaaga | 1560 |
| attttgccct taagtttaaa acaatccttg atttttttct tttaatatcc tccctatcaa | 1620 |
| tcaccgaact acttttcttt ttaaagtact tggttaagta atacttttct gaggattggt | 1680 |
| tagatattgt caaatatttt tgctggtcac ctaaaatgca gccagatgtt tcattgttaa | 1740 |
| aaatctatat aaaagtgcaa gctgcctttt ttaaattaca taaatcccat gaatacatgg | 1800 |
| ccaaaatagt tattttttaa agactttaaa ataaatgatt aatcgatgct | 1850 |

The invention is further described by the following numbered paragraphs:

1. A crystallisable composition comprising a substantially pure protein with at least 37% amino acid sequence identity to the amino acid sequence of rat DPPI protein as shown in SEQ ID NO: 1.

2. A crystallised molecule or molecular complex comprising a rat DPPI protein with the amino acid sequence as shown in SEQ ID NO: 1.

3. A crystallised molecule or molecular complex comprising a protein with at least 37% amino acid sequence identity to the amino acid sequence of rat DPPI protein as shown in SEQ ID NO: 1.

4. A crystallised molecule or molecular complex according to paragraph 3 comprising a protein with at least 75% amino acid sequence identity to the amino acid sequence of rat DPPI protein.

5. A crystallised molecule or molecular complex according to paragraphs 3 or 4, comprising a protein, characterised by a space group P6₄₂₂ and unit cell dimensions a=166.24 Å, b=166.24 Å, c=80.48 Å with α=β=90° and γ=120°

6. A crystallised molecule or molecular complex according to any of paragraphs 3-5, comprising all or any parts of a binding pocket defined by a negative charge in the active site cleft of a cysteine peptidase by the side chain of the N-terminal residue of a residual pro-part.

7. A crystallised molecule or molecular complex according to paragraph 6, wherein the free amino group of a conserved Asp1 is held in position by a hydrogen bond to the backbone carbonyl oxygen atom of Asp274.

8. A crystallised molecule or molecular complex according to paragraph 7, further characterised by the delocalised negative charge that said residue carries under physiological conditions on its OD1 and OD2 oxygen atoms which are localised about 7-9 Å from the sulphur atom of the catalytic Cys233 residue.

9. A crystallised molecule or molecular complex according to any of paragraphs 3-8 wherein the position of a N-terminal Asp1 residue is fixed by a hydrogen bond between the free amino group of this residue (hydrogen bond donor) and the backbone carbonyl oxygen of Asp274 (hydrogen bond acceptor).

10. A crystallised molecule or molecular complex according to any of paragraphs 3-9, in which said protein is a DPPI or DPPI-like protein.

11. A crystallised molecule or molecular complex according to any of paragraphs 3-10, in which said molecule is mutated prior to being crystallised.

12. A crystallised molecule or molecular complex according to any of paragraphs 3-11, in which said molecule is chemically modified.

13. A crystallised molecule or molecular complex according to any of paragraphs 3-11, in which said molecule is enzymatically modified.

14. A crystallised molecular complex according to any of paragraphs 3-13, which is in a covalent or non-covalent association with at least one other molecule or molecular complex.

15. A crystallised molecular complex according to any of paragraphs 2-14, which is complexed with a co-factor.

16. A crystallised molecular complex according to any of paragraphs 2-15, which is complexed with a halide.

17. A crystallised molecular complex according to paragraph 16, which is complexed with a chloride.

18. A heavy atom derivative of a crystallised molecule or molecular complex according to any of paragraphs 2-17.

19. The crystal structure of a protein with at least 37% amino acid sequence identity to the amino acid sequence of rat DPPI protein as shown in SEQ ID NO: 1.

20. The crystal structure of a protein with at least 75% amino acid sequence identity to the amino acid sequence of rat DPPI protein as shown in SEQ ID NO: 1.

21. The crystal structure of a protein with an amino acid sequence as shown in SEQ ID NO: 1.

22. The crystal structure of a protein for which the structural co-ordinates of the back bone nitrogen, alpha-carbon and carbonyl carbon atoms of said protein have a root-mean-square deviation from the structural co-ordinates of the equivalent back bone atoms of rat DPPI (as defined in Table 2) of less than 2 Å following structural alignment of equivalent back bone atoms.

23. The crystal structure of a protein according to any of paragraphs 19-22, in which said protein has been mutated priorto being crystallised.

24. The crystal structure of a protein according to any of paragraphs 19-23, in which said protein is chemically modified.

25. The crystal structure of a protein according to any of paragraphs 19-23, in which said protein is enzymatically modified.

26. The crystal structure of a protein according to any of paragraphs 19-25, in which said protein is in a covalent or non-covalent association with at least one other atom, molecule, or molecular complex.

27. The crystal structure of a protein according to any of paragraphs 19-26, in which said protein is complexed with a co-factor.

28. The crystal structure of a protein according to any of paragraphs 19-27, in which said protein is complexed with a halide.

29. The crystal structure of a protein according to paragraph 28, in which said protein is complexed with chloride.

30. A crystal structure of a heavy atom derivative of a protein according to any of paragraphs 19-29.

31. The structural co-ordinates of a protein with at least 37% amino acid sequence identity to the amino acid sequence of rat DPPI protein as shown in SEQ ID NO: 1, that has been found by homology modelling characterised by using any structure co-ordinates of a crystal structure according to any of paragraphs 19-30.

32. A method for producing a crystallised molecule or molecular complex according to any of paragraphs 2-19, characterised by obtaining a sufficient amount of sufficiently pure protein characterised by employing a baculovirus/insect cell system.

33. A method for producing a crystallised molecule or molecular complex according to paragraph 29, further characterised by using 12 mg/ml protein in a reservoir solution containing 1.4 M (NH₄)₂SO₄, 0.1 M bis-tris propane pH 7.5 and 10% PEG 8000.

34. A method for determining a crystal structure of a first protein structurally related to a second protein with a known crystal structure or structural co-ordinates according to any of paragraphs 19-31, characterised by applying any structural co-ordinates of said known crystal structure for determining phases of diffraction data, obtained by X-ray analysis of said crystal of said first protein, by the method of molecular replacement analysis.

35. A method for theoretically modelling the structure of a first protein with at least 37% amino acid sequence identity to the amino acid sequence of rat DPPI protein as shown in SEQ ID NO: 1, characterised by a) aligning the sequence of said first protein with the sequence of a second protein with known crystal structure or structural co-ordinates according to any of paragraphs 19-31, and incorporating the first sequence into the structure of the second polypeptide, thereby creating a preliminary structural model of said first protein, b) subjecting said preliminary structural model to energy minimisation, resulting in an energy minimised model, c) remodelling the regions of said energy minimised model where stereochemistry restraints are violated, and d) obtaining structure co-ordinates of the final model.

36. A method for selecting, testing and/or rationally or semi-rationally designing a chemical compound which binds covalently or non-covalently to a protein with at least 37% amino acid sequence identity to the amino acid sequence of rat DPPI protein as shown in SEQ ID NO: 1, characterised by applying in a computational analysis structure co-ordinates of a crystal structure according to any of paragraphs 19-31 and/or 35.

37. A method for identifying a potential inhibitor of an enzyme with at least 37% amino acid sequence identity to the amino acid sequence of rat DPPI protein as shown in SEQ ID NO: 1, comprising the following steps: a) using the atomic co-ordinates of a crystallised molecule or molecular complex according to any of paragraphs 2-19 to define the catalytic active sites and/or an accessory binding site of said enzyme, b) identifying a compound that fits the active site and/or an accessory binding site of a), c) obtaining the compound, and d) contacting the compound with a DPPI or DPPI-like protein to determine the binding properties and/or effects of said compound on and/or the inhibition of the enzymatic activity of DPPI by said compound.

38. A method for identifying a potential inhibitor according to paragraph 37, wherein the atomic co-ordinates of said crystallised molecule or molecular complex are obtained by X-ray diffraction studies using a crystallised molecule or molecular complex according to any of paragraphs 2-19.

39. A method for identifying a potential inhibitor of a DPPI or DPPI-like protein comprising the following steps: a) using all or some of the atomic co-ordinates of a crystal structure according to paragraphs 19-30 to define the catalytic active sites or accessory binding sites of an enzyme with at least 37% amino acid sequence identity to the amino acid sequence of rat DPPI protein as shown in SEQ ID NO: 1, b) identifying a compound that fits the active site or accessory binding site of a), c) obtaining the compound, and d) contacting the compound with a DPPI or DPPI-like protein in the presence of a substrate in solution to determine the inhibition of the enzymatic activity by said compound.

40. A method for identifying a potential inhibitor of a DPPI or DPPI-like protein comprising the following steps: a) using all or some of the structural co-ordinates of a protein according to paragraph 31 to define the catalytic active sites or accessory binding sites of an enzyme with at least 37% amino acid sequence identity to the amino acid sequence of rat DPPI protein as shown in SEQ ID NO: 1, b) identifying a compound that fits the active site or accessory binding site of a), c) obtaining the compound, and d) contacting the compound with a DPPI or DPPI-like protein in the presence of a substrate in solution to determine the inhibition of the enzymatic activity by said compound.

41. A method for designing a potential inhibitor of a DPPI or DPPI-like protein comprising the steps of a) providing a three dimensional model of the receptor site in an enzyme with at least 37% amino acid sequence identity to the amino acid sequence of rat DPPI protein as shown in SEQ ID NO: 1 and a known inhibitor, b) locating the conserved residues in the known inhibitor which constitute the inhibition binding pocket, c) designing a new a DPPI or DPPI-like protein inhibitor, which possesses complementary structural features and binding forces to the residues in the known inhibitor's inhibition binding pocket.

42. A method according to paragraph 41, wherein the three-dimensional model of a protein with at least 37% amino acid sequence identity to the amino acid sequence of rat DPPI protein as shown in SEQ ID NO: 1 in step a) is the model set out in FIG. 3.

43. A method according to paragraphs 41 or 42 wherein said three-dimensional model is constructed on structural co-ordinates obtained from a crystal structure according to paragraphs 19-30 or on structural co-ordinates of a protein according to paragraph 31.

44. A method according to any of paragraph 36-43, wherein said identified compound and/or potential inhibitor is designed de novo.

45. A method according to any of paragraph 36-43, wherein said identified compound and/or potential inhibitor is designed from a known inhibitor or from a fragment capable of associating with a DPPI or DPPI-like protein.

46. A method according to paragraph 45, wherein said known inhibitor is selected from the group consisting of dipeptide halomethyl ketone inhibitors, dipeptide diazomethyl ketone inhibitors, dipeptide dimethylsulphonium salt inhibitors, dipeptide nitril inhibitors, dipeptide alpha-keto carboxylic acid inhibitors, dipeptide alpha-keto ester inhibitors, dipeptide alpha-keto amide inhibitors, dipeptide alpha-diketone inhibitors, dipeptide acyloxymethyl ketone inhibitors, dipeptide aldehyde inhibitors and dipeptide epoxysuccinyl inhibitors.

47. A method according to any of paragraphs 36-46, wherein said step of employing said structural co-ordinates to design, or select said potential inhibitor comprises the steps of: a) identifying chemical entities or fragments capable of associating with a protein with at least 37% amino acid sequence identity to the amino acid sequence of rat DPPI protein as shown in SEQ ID NO: 1, and b) assembling the identified chemical entities or fragments into a single 48. A chemical compound and/or potential inhibitor identified by a method according to any of paragraphs 36-47.

49. A chemical compound and/or potential inhibitor identifiable by a method according to any of paragraphs 36-47.

50. A potential inhibitor, which possesses a positive charge that forms a salt bridge to the negative charge on the side chain of a conserved Asp1 and/or Asp274 of a protein with at least 37% amino acid sequence identity to the amino acid sequence of rat DPPI protein as shown in SEQ ID NO: 1

51. Use of any of the atomic co-ordinates according to paragraphs 31 and/or 35 and/or the atomic co-ordinates of a crystal structure according to paragraphs 19-30 for the identification of a potential inhibitor of a DPPI or DPPI-like protein.

52. A method for selecting, testing and/or rationally or semi-rationally designing a modified protein with at least 37% amino acid sequence identity to the amino acid sequence of rat DPPI protein as shown in SEQ. ID. NO. 1, characterised by applying any of the atomic co-ordinates according to paragraphs 31 and/or 35, and/or the atomic co-ordinates of a crystal structure according to any of the paragraphs 19-30.

53. Use of any of the atomic co-ordinates according to paragraphs 31 and/or 35 and/or the atomic co-ordinates of a crystal structure according to any of paragraphs 19-30 for the modification of a protein with at least 37% amino acid sequence identity to the amino acid sequence of rat DPPI protein as shown in SEQ. ID. NO. 1, such that it can catalyse the cleavage of a natural, unnatural or synthetic substrate more efficiently than the wild type enzyme.

54. Use according to paragraph 53, wherein such substrates are selected from the group consisting of dipeptide amides and esters, dipeptides C-terminally linked to a chromogenic or fluorogenic group, polyhistidine purification tags and granule serine proteases with a natural dipeptide propeptide extension.

55. A modified protein obtained by a method or use according to any of paragraphs 52-54.

56. A modified protein obtainable by a method or use according to any of paragraphs 52-54.

57. Use of a chemical compound, potential inhibitor, or modified protein according to any of paragraphs 48-50, 55 or 56, respectively, for interfering with a DPPI catalysed activation of a mammalian tryptase.

58. Use of a chemical compound, potential inhibitor, or modified protein according to any of paragraphs 48-50, 55 or 56, respectively, for interfering with a DPPI catalysed activation of a human tryptase.

59. Use of a chemical compound, potential inhibitor or modified protein according to any of paragraphs 48-50, 55 or 56, respectively, for interfering with a DPPI catalysed activation of a mammalian chymase.

60. Use of a chemical compound, potential inhibitor, or modified protein according to any of paragraphs 48-50, 55 or 56, respectively, for interfering with a DPPI catalysed activation of a human chymase.

61. Use according to any of paragraphs 57-60, for treating a mast cell related disease by interfering with a DPPI catalysed activation of mast cell tryptase and/or mast cell chymase. ulcerative colitis and Crohn's disease and asthma psoreasis

62. Use of a chemical compound, potential inhibitor, or modified protein according to any of paragraphs 48-50, 55 or 56, respectively, for treating a disease related to excessive and/or reduced apoptosis.

63. Use of a chemical compound, potential inhibitor, or modified protein according to any of paragraphs 48-50, 55 or 56, respectively, for treating a granzyme related disease by interfering with the DPPI catalysed activation of a granzyme.

64. Use according to paragraph 62 or 63, by interfering with a DPPI catalysed activation of a granzyme selected from the group consisting of granzyme A, B, H, K or M.

65. Use according to any of paragraphs 62-64, wherein said disease is selected from the group consisting of cancer.

66. Use of a chemical compound, potential inhibitor, or modified protein according to any of paragraphs 48-50, 55 or 56, respectively, for treating a disease related to excessive and/or reduced proteolysis.

67. Use according to paragraph 66, characterised by interfering with a DPPI catalysed activation of cathepsin G and/or leukocyte elastase.

68. Use according to paragraph 67, wherein said disease is selected from the group consisting of lung emphysema, cystic fibrosis, adult respiratory distress syndrome, rheumatoid arthritis and infectious diseases.

69. Use of a chemical compound, potential inhibitor or modified protein according to any of paragraphs 48-50, 55 or 56, respectively, for manufacturing of a pharmaceutical composition for the treatment of a disease related to dysfunctional or anomalous DPPI activation of one or more human serine proteases.

70. Use according to paragraph 69, wherein said human serine protease is selected from the group consisting of tryptase, chymase, granzymes A, B, H, K and M, cathepsin G and leukocyte elastase.

71. Use of a chemical compound, potential inhibitor or modified protein according to any of paragraphs 48-50, 55 or 56, respectively, for the manufacturing of a pharmaceutical composition for the treatment of a mast cell related disease, characterised by dysfunctional and/or anomalous DPPI activation of a human tryptase and/or chymase.

72. Use of a chemical compound, potential inhibitor or modified protein according to any of paragraphs 48-50, 55 or 56, respectively, for the manufacturing of a pharmaceutical composition for the treatment of a disease related to excessive or reduced granzyme activity resulting from dys-functional or anomalous DPPI activation.

73. Use of a chemical compound, potential inhibitor, or modified protein according to any of paragraphs 48-50, 55 or 56, respectively, for the manufacturing of a pharmaceutical composition for the treatment of a disease related to excessive or reduced proteolysis by cathepsin G and/or leukocyte elastase.

74. A pharmaceutical composition comprising a chemical compound, potential inhibitor, or modified protein according to any of paragraphs 48-50, 55 or 56, respectively.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

Each patent, patent application, and publication cited or described in the present application is hereby incorporated by reference in its entirety as if each individual patent, patent application, or publication was specifically and individually indicated to be incorporated by reference.

LENGTHY TABLES
The patent application contains a lengthy table section. A copy of the table is available in electronic form from the USPTO web site (http://seqdata.uspto.gov/?pageRequest=docDetail&DocID=US20110236367A1). An electronic copy of the table will also be available from the USPTO upon request and payment of the fee set forth in 37 CFR 1.19(b)(3).

What is claimed is:

1. A crystallisable composition comprising a substantially pure protein with at least 37% amino acid sequence identity to the amino acid sequence of rat DPPI protein as shown in SEQ ID NO: 1.

2. A crystallised molecule or molecular complex comprising a rat DPPI protein with the amino acid sequence as shown in SEQ ID NO: 1.

3. A crystallised molecule or molecular complex comprising a protein with at least 37% amino acid sequence identity to the amino acid sequence of rat DPPI protein as shown in SEQ ID NO: 1.

4. A crystallised molecule or molecular complex according to claim 3 comprising a protein with at least 75% amino acid sequence identity to the amino acid sequence of rat DPPI protein.

5. A crystallised molecule or molecular complex according to claim 3 or 4, comprising a protein, characterised by a space group P6₄₂₂ and unit cell dimensions a=166.24 Å, b=166.24 Å, c=80.48 Åwith α=β=90° and γ=120°

6. A crystallised molecule or molecular complex according to any of claims 3-5, comprising all or any parts of a binding pocket defined by a negative charge in the active site cleft of a cysteine peptidase by the side chain of the N-terminal residue of a residual pro-part.

7. A crystallised molecule or molecular complex according to claim 6, wherein the free amino group of a conserved Asp1 is held in position by a hydrogen bond to the backbone carbonyl oxygen atom of Asp274.

8. A crystallised molecule or molecular complex according to claim 7, further characterised by the delocalised negative charge that said residue carries under physiological conditions on its OD1 and OD2 oxygen atoms which are localised about 7-9 Å from the sulphur atom of the catalytic Cys233 residue.

9. A crystallised molecule or molecular complex according to any of claims 3-8 wherein the position of a N-terminal Asp1 residue is fixed by a hydrogen bond between the free amino group of this residue (hydrogen bond donor) and the backbone carbonyl oxygen of Asp274 (hydrogen bond acceptor).

10. A crystallised molecule or molecular complex according to any of claims 3-9, in which said protein is a DPPI or DPPI-like protein.

11. A crystallised molecule or molecular complex according to any of claims 3-10, in which said molecule is mutated prior to being crystallised.

12. A crystallised molecule or molecular complex according to any of claims 3-11, in which said molecule is chemically modified.

13. A crystallised molecule or molecular complex according to any of claims 3-11, in which said molecule is enzymatically modified.

14. A crystallised molecular complex according to any of claims 3-13, which is in a covalent or non-covalent association with at least one other molecule or molecular complex.

15. A crystallised molecular complex according to any of claims 2-14, which is complexed with a co-factor.

16. A crystallised molecular complex according to any of claims 2-15, which is complexed with a halide.

17. A crystallised molecular complex according to claim 16, which is complexed with a chloride.

18. A heavy atom derivative of a crystallised molecule or molecular complex according to any of claims 2-17.

19. The crystal structure of a protein with at least 37% amino acid sequence identity to the amino acid sequence of rat DPPI protein as shown in SEQ ID NO: 1.

20. The crystal structure of a protein with at least 75% amino acid sequence identity to the amino acid sequence of rat DPPI protein as shown in SEQ ID NO: 1.

21. The crystal structure of a protein with an amino acid sequence as shown in SEQ ID NO: 1.

22. The crystal structure of a protein for which the structural co-ordinates of the back bone nitrogen, alpha-carbon and carbonyl carbon atoms of said protein have a root-mean-square deviation from the structural co-ordinates of the equivalent back bone atoms of rat DPPI (as defined in Table 2) of less than 2 Å following structural alignment of equivalent back bone atoms.

23. The crystal structure of a protein according to any of claims 19-22, in which said protein has been mutated priorto being crystallised.

24. The crystal structure of a protein according to any of claims 19-23, in which said protein is chemically modified.

25. The crystal structure of a protein according to any of claims 19-23, in which said protein is enzymatically modified.

26. The crystal structure of a protein according to any of claims 19-25, in which said protein is in a covalent or non-covalent association with at least one other atom, molecule, or molecular complex.

27. The crystal structure of a protein according to any of claims 19-26, in which said protein is complexed with a co-factor.

28. The crystal structure of a protein according to any of claims 19-27, in which said protein is complexed with a halide.

29. The crystal structure of a protein according to claim 28, in which said protein is complexed with chloride.

30. A crystal structure of a heavy atom derivative of a protein according to any of claims 19-29.

31. The structural co-ordinates of a protein with at least 37% amino acid sequence identity to the amino acid sequence of rat DPPI protein as shown in SEQ ID NO: 1, that has been found by homology modelling characterised by using any structure co-ordinates of a crystal structure according to any of claims 19-30.

32. A method for producing a crystallised molecule or molecular complex according to any of claims 2-19, characterised by obtaining a sufficient amount of sufficiently pure protein characterised by employing a baculovirus/insect cell system.

33. A method for producing a crystallised molecule or molecular complex according to claim 29, further characterised by using 12 mg/ml protein in a reservoir solution containing 1.4 M (NH₄)₂SO₄, 0.1 M bis-tris propane pH 7.5 and 10% PEG 8000.

34. A method for determining a crystal structure of a first protein structurally related to a second protein with a known crystal structure or structural co-ordinates according to any of claims 19-31, characterised by applying any structural co-ordinates of said known crystal structure for determining phases of diffraction data, obtained by X-ray analysis of said crystal of said first protein, by the method of molecular replacement analysis.

35. A method for theoretically modelling the structure of a first protein with at least 37% amino acid sequence identity to the amino acid sequence of rat DPPI protein as shown in SEQ ID NO: 1, characterised by a) aligning the sequence of said first protein with the sequence of a second protein with known crystal structure or structural co-ordinates according to any of claims 19-31, and incorporating the first sequence into the structure of the second polypeptide, thereby creating a preliminary structural model of said first protein, b) subjecting said preliminary structural model to energy minimisation, resulting in an energy minimised model, c) remodelling the regions of said energy minimised model where stereochemistry restraints are violated, and d) obtaining structure co-ordinates of the final model.

36. A method for selecting, testing and/or rationally or semi-rationally designing a chemical compound which binds covalently or non-covalently to a protein with at least 37% amino acid sequence identity to the amino acid sequence of rat DPPI protein as shown in SEQ ID NO: 1, characterised by applying in a computational analysis structure co-ordinates of a crystal structure according to any of claims 19-31 and/or 35.

37. A method for identifying a potential inhibitor of an enzyme with at least 37% amino acid sequence identity to the amino acid sequence of rat DPPI protein as shown in SEQ ID NO: 1, comprising the following steps: a) using the atomic co-ordinates of a crystallised molecule or molecular complex according to any of claims 2-19 to define the catalytic active sites and/or an accessory binding site of said enzyme, b) identifying a compound that fits the active site and/or an accessory binding site of a), c) obtaining the compound, and d) contacting the compound with a DPPI or DPPI-like protein to determine the binding properties and/or effects of said compound on and/or the inhibition of the enzymatic activity of DPPI by said compound.

38. A method for identifying a potential inhibitor according to claim 37, wherein the atomic co-ordinates of said crystallised molecule or molecular complex are obtained by X-ray diffraction studies using a crystallised molecule or molecular complex according to any of claims 2-19.

39. A method for identifying a potential inhibitor of a DPPI or DPPI-like protein comprising the following steps: a) using all or some of the atomic co-ordinates of a crystal structure according to claims 19-30 to define the catalytic active sites or accessory binding sites of an enzyme with at least 37% amino acid sequence identity to the amino acid sequence of rat DPPI protein as shown in SEQ ID NO: 1, b) identifying a compound that fits the active site or accessory binding site of a), c) obtaining the compound, and d) contacting the compound with a DPPI or DPPI-like protein in the presence of a substrate in solution to determine the inhibition of the enzymatic activity by said compound.

40. A method for identifying a potential inhibitor of a DPPI or DPPI-like protein comprising the following steps: a) using all or some of the structural co-ordinates of a protein according to claim 31 to define the catalytic active sites or accessory binding sites of an enzyme with at least 37% amino acid sequence identity to the amino acid sequence of rat DPPI protein as shown in SEQ ID NO: 1, b) identifying a compound that fits the active site or accessory binding site of a), c) obtaining the compound, and d) contacting the compound with a DPPI or DPPI-like protein in the presence of a substrate in solution to determine the inhibition of the enzymatic activity by said compound.

41. A method for designing a potential inhibitor of a DPPI or DPPI-like protein comprising the steps of: a) providing a three dimensional model of the receptor site in an enzyme with at least 37% amino acid sequence identity to the amino acid sequence of rat DPPI protein as shown in SEQ ID NO: 1 and a known inhibitor, b) locating the conserved residues in the known inhibitor which constitute the inhibition binding pocket, c) designing a new a DPPI or DPPI-like protein inhibitor, which possesses complementary structural features and binding forces to the residues in the known inhibitor's inhibition binding pocket.

42. A method according to claim 41, wherein the three-dimensional model of a protein with at least 37% amino acid sequence identity to the amino acid sequence of rat DPPI protein as shown in SEQ ID NO: 1 in step a) is the model set out in FIG. 3.

43. A method according to claim 41 or 42 wherein said three-dimensional model is constructed on structural co-ordinates obtained from a crystal structure according to claims 19-30 or on structural co-ordinates of a protein according to claim 31.

44. A method according to any of claim 36-43, wherein said identified compound and/or potential inhibitor is designed de novo.

45. A method according to any of claim 36-43, wherein said identified compound and/or potential inhibitor is designed from a known inhibitor or from a fragment capable of associating with a DPPI or DPPI-like protein.

46. A method according to claim 45, wherein said known inhibitor is selected from the group consisting of dipeptide halomethyl ketone inhibitors, dipeptide diazomethyl ketone inhibitors, dipeptide dimethylsulphonium salt inhibitors, dipeptide nitril inhibitors, dipeptide alpha-keto carboxylic acid inhibitors, dipeptide alpha-keto ester inhibitors, dipeptide alpha-keto amide inhibitors, dipeptide alpha-diketone inhibitors, dipeptide acyloxymethyl ketone inhibitors, dipeptide aldehyde inhibitors and dipeptide epoxysuccinyl inhibitors.

47. A method according to any of claims 36-46, wherein said step of employing said structural co-ordinates to design, or select said potential inhibitor comprises the steps of: a) identifying chemical entities or fragments capable of associating with a protein with at least 37% amino acid sequence identity to the amino acid sequence of rat DPPI protein as shown in SEQ ID NO: 1, and b) assembling the identified chemical entities or fragments into a single 48. A chemical compound and/or potential inhibitor identified by a method according to any of claims 36-47.

49. A chemical compound and/or potential inhibitor identifiable by a method according to any of claims 36-47.

50. A potential inhibitor, which possesses a positive charge that forms a salt bridge to the negative charge on the side chain of a conserved Asp1 and/or Asp274 of a protein with at least 37% amino acid sequence identity to the amino acid sequence of rat DPPI protein as shown in SEQ ID NO: 1

51. Use of any of the atomic co-ordinates according to claims 31 and/or 35 and/or the atomic co-ordinates of a crystal structure according to claims 19-30 for the identification of a potential inhibitor of a DPPI or DPPI-like protein.

52. A method for selecting, testing and/or rationally or semi-rationally designing a modified protein with at least 37% amino acid sequence identity to the amino acid sequence of rat DPPI protein as shown in SEQ. ID. NO. 1, characterised by applying any of the atomic co-ordinates according to claims 31 and/or 35, and/or the atomic co-ordinates of a crystal structure according to any of the claims 19-30.

53. Use of any of the atomic co-ordinates according to claims 31 and/or 35 and/or the atomic co-ordinates of a crystal structure according to any of claims 19-30 for the modification of a protein with at least 37% amino acid sequence identity to the amino acid sequence of rat DPPI protein as shown in SEQ. ID. NO. 1, such that it can catalyse the cleavage of a natural, unnatural or synthetic substrate more efficiently than the wild type enzyme.

54. Use according to claim 53, wherein such substrates are selected from the group consisting of dipeptide amides and esters, dipeptides C-terminally linked to a chromogenic or fluorogenic group, polyhistidine purification tags and granule serine proteases with a natural dipeptide propeptide extension.

55. A modified protein obtained by a method or use according to any of claims 52-54.

56. A modified protein obtainable by a method or use according to any of claims 52-54.

57. Use of a chemical compound, potential inhibitor, or modified protein according to any of claim 48-50, 55 or 56, respectively, for interfering with a DPPI catalysed activation of a mammalian tryptase.

58. Use of a chemical compound, potential inhibitor, or modified protein according to any of claim 48-50, 55 or 56, respectively, for interfering with a DPPI catalysed activation of a human tryptase.

59. Use of a chemical compound, potential inhibitor or modified protein according to any of claim 48-50, 55 or 56, respectively, for interfering with a DPPI catalysed activation of a mammalian chymase.

60. Use of a chemical compound, potential inhibitor, or modified protein according to any of claim 48-50, 55 or 56, respectively, for interfering with a DPPI catalysed activation of a human chymase.

61. Use according to any of claims 57-60, for treating a mast cell related disease by interfering with a DPPI catalysed activation of mast cell tryptase and/or mast cell chymase. ulcerative colitis and Crohn's disease and asthma psoreasis

62. Use of a chemical compound, potential inhibitor, or modified protein according to any of claim 48-50, 55 or 56, respectively, for treating a disease related to excessive and/or reduced apoptosis.

63. Use of a chemical compound, potential inhibitor, or modified protein according to any of claim 48-50, 55 or 56, respectively, for treating a granzyme related disease by interfering with the DPPI catalysed activation of a granzyme.

64. Use according to claim 62 or 63, by interfering with a DPPI catalysed activation of a granzyme selected from the group consisting of granzyme A, B, H, K or M.

65. Use according to any of claims 62-64, wherein said disease is selected from the group consisting of cancer.

66. Use of a chemical compound, potential inhibitor, or modified protein according to any of claim 48-50, 55 or 56, respectively, for treating a disease related to excessive and/or reduced proteolysis.

67. Use according to claim 66, characterised by interfering with a DPPI catalysed activation of cathepsin G and/or leukocyte elastase.

68. Use according to claim 67, wherein said disease is selected from the group consisting of lung emphysema, cystic fibrosis, adult respiratory distress syndrome, rheumatoid arthritis and infectious diseases.

69. Use of a chemical compound, potential inhibitor or modified protein according to any of claim 48-50, 55 or 56, respectively, for manufacturing of a pharmaceutical composition for the treatment of a disease related to dysfunctional or anomalous DPPI activation of one or more human serine proteases.

70. Use according to claim 69, wherein said human serine protease is selected from the group consisting of tryptase, chymase, granzymes A, B, H, K and M, cathepsin G and leukocyte elastase.

71. Use of a chemical compound, potential inhibitor or modified protein according to any of claim 48-50, 55 or 56, respectively, for the manufacturing of a pharmaceutical composition for the treatment of a mast cell related disease, characterised by dysfunctional and/or anomalous DPPI activation of a human tryptase and/or chymase.

72. Use of a chemical compound, potential inhibitor or modified protein according to any of claim 48-50, 55 or 56, respectively, for the manufacturing of a pharmaceutical composition for the treatment of a disease related to excessive or reduced granzyme activity resulting from dys-functional or anomalous DPPI activation.

73. Use of a chemical compound, potential inhibitor, or modified protein according to any of claim 48-50, 55 or 56, respectively, for the manufacturing of a pharmaceutical composition for the treatment of a disease related to excessive or reduced proteolysis by cathepsin G and/or leukocyte elastase.

74. A pharmaceutical composition comprising a chemical compound, potential inhibitor, or modified protein according to any of claim 48-50, 55 or 56, respectively.


SEQUENCE LISTING (12 sequences)

SEQ ID NO: 1 - protein, 462 aa, Rattus norvegicus
MGPWTHSLRAALLLVLLGVCTVSSDTPANCTYPDLLGTWVFQVGPRHPRSHINCSVMEPTEEKVVIHLKKLDTAYDEVGNSGYFTLIYNQGFEIVLNDYKWFAFFKYEVKGSRAISYCHETMTGWVHDVLGRNWACFVGKKMANHSEKVYVNVAHLGGLQEKYSERLYSHNHNFVKAINSVQKSWTATTYEEYEKLSIRDLIRRSGHSGRILRPKPAPITDEIQQQILSLPESWDWRNVRGINFVSPVRNQESCGSCYSFASLGMLEARIRILTNNSQTPILSPQEVVSCSPYAQGCDGGFPYLIAGKYAQDFGVVEENCFPYTATDAPCKPKENCLRYYSSEYYYVGGFYGGCNEALMKLELVKHGPMAVAFEVHDDFLHYHSGIYHHTGLSDPFNPFELTNHAVLLVGYGKDPVTGLDYWIVKNSWGSQWGESGYFRIRRGTDECAIESIAMAAIPIPKL

SEQ ID NO: 2 - DNA, 1850 bp, Rattus norvegicus
gaattccggttctagttgttgttttctctgccatctgctctccgggcgccgtcaaccatgggtccgtggacccactccttgcgcgccgccctgctgctggtgcttttgggagtctgcaccgtgagctccgacactcctgccaactgcacttaccctgacctgctgggtacctgggttttccaggtgggccctagacatccccgaagtcacattaactgctcggtaatggaaccaacagaagaaaaggtagtgatacacctgaagaagttggatactgcctatgatgaagtgggcaattctgggtatttcaccctcatttacaaccaaggctttgagattgtgttgaatgactacaagtggtttgcgtttttcaagtatgaagtcaaaggcagcagagccatcagttactgccatgagaccatgacagggtgggtccatgatgtcctgggccggaactgggcttgctttgttggcaagaagatggcaaatcactctgagaaggtttatgtgaatgtggcacaccttggaggtctccaggaaaaatattctgaaaggctctacagtcacaaccacaactttgtgaaggccatcaattctgttcagaagtcttggactgcaaccacctatgaagaatatgagaaactgagcatacgagatttgataaggagaagtggccacagcggaaggatcctaaggcccaaacctgccccgataactgatgaaatacagcaacaaattttaagtttgccagaatcttgggactggagaaacgtccgtggcatcaattttgttagccctgttcgaaaccaagaatcttgtggaagctgctactcatttgcctctctgggtatgctagaagcaagaattcgtatattaaccaacaattctcagaccccaatcctgagtcctcaggaggttgtatcttgtagcccgtatgcccaaggttgtgatggtggattcccatacctcattgcaggaaagtatgcccaagattttggggtggtggaagaaaactgctttccctacacagccacagatgctccatgcaaaccaaaggaaaactgcctccgttactattcttctgagtactactatgtgggtggtttctatggtggctgcaatgaagccctgatgaagcttgagctggtcaaacacggacccatggcagttgcctttgaagtccacgatgacttcctgcactaccacagtgggatctaccaccacactggactgagcgaccctttcaacccctttgagctgaccaatcatgctgttctgcttgtgggctatggaaaagatccagtcactgggttagactactggattgtcaagaacagctggggctctcaatggggtgagagtggctacttccggatccgcagaggaactgatgaatgtgcaattgagagtatagccatggcagccataccgattcctaaattgtaggacctagctcccagtgtcccatacagctttttattattcacagggtgatttagtcacaggctggagacttttacaaagcaatatcagaagcttaccactaggtacccttaaagaattttgcccttaagtttaaaacaatccttgatttttttcttttaatatcctccctatcaatcaccgaactacttttctttttaaagtacttggttaagtaatacttttctgaggattggttagatattgtcaaatatttttgctggtcacctaaaatgcagccagatgtttcattgttaaaaatctatataaaagtgcaagctgccttttttaaattacataaatcccatgaatacatggccaaaatagttattttttaaagactttaaaataaatgattaatcgatgct

SEQ ID NO: 3 - protein, 439 aa, Homo sapiens
DTPANCTYLDLLGTWVFQVGSSGSQRDVNCSVMGPQEKKVVVYLQKLDTAYDDLGNSGHFTIIYNQGFEIVLNDYKWFAFFKYKEEGSKVTTYCNETMTGWVHDVLGRNWACFTGKKVGTASENVYVNTAHLKNSQEKYSNRLYKYDHNFVKAINAIQKSWTATTYMEYETLTLGDMIRRSGGHSRKIPRPKPAPLTAEIQQKILHLPTSWDWRNVHGINFVSPVRNQASCGSCYSFASMGMLEARIRILTNNSQTPILSPQEVVSCSQYAQGCEGGFPYLIAGKYAQDFGLVEEACFPYTGTDSPCKMKEDCFRYYSSEYHYVGGFYGGCNEALMKLELVHHGPMAVAFEVYDDFLHYKKGIYHHTGLRDPFNPFELTNHAVLLVGYGTDSASGMDYWIVKNSWGTGWGENGYFRIRRGTDECAIESIAVAATPIPKL

SEQ ID NO: 4 - protein, 435 aa, dog
DTPANCTHPELLGTWVFQVGPAGSRSVNCSCMGPPEKKVVVHLEKLDTAYDNFGNTGHFTIIYNQGFEIVLNDYKWFAFFKYKEEGHKVTSYCNETMTGWVHDVLGRNWACFTGTKMGTTSEKAKVNTKHIERLQENNSNRLYKYNYEFVKAINTIQKSWTATRYIEYETLTLRDMMTRVGGRKIPRPKPTPLTAEIHEEISRLPTSWDWRNVRGTNFVSPVRNQASCGSCYAFASTAMLEARIRILTNNTQTPILSPQEIVSCSQYAQGCEGGFPYLIAGKYAQDFGLVEEACFPYAGSDSPCKPNDCFRYYSSEYYYVGGFYGACNEALMKLELVRHGPMAVAFEVYDDFFHYQKGIYYHTGLRDPFNPFELTNHAVLLVGYGTDSASGMDYWIVKNSWGSRWGEDGYFRIRRGTDECAIESIAVAATPIPKL

SEQ ID NO: 5 - protein, 439 aa, bovine
DTPANCTYPDLLGTWVFQVGSSGSQRDVNCSVMGPPEKKVVVHLKKLDTAYDDFGNSGHFTIIYNQGFEIVLNDYKWFAFFKYKEEGGKVTSYCHETMTGWVHDVLGRNWACFTGRKTGNTSENVNVNTARLAGLEETYSNRLYRYNGDFVKAINAIQKSWTAAPYMEYETLTLKEMIRRGGGHSRRIPRPKPAPITAEIQKKILHLPTSWDWRNVHGINFVTPVRNQGSCGSCYSFASMGMMEARIRILTNNTQTPILSPQEVVSCSQYAQGCEGGFPYLIAGKYAQDFGLVEEDCFPYTGTDSPCRLKEGCFRYYSSEYHYVGGFYGGCNEALMKLELVHQGPMAVAFEVYDDFLHYRKGVYHHTGLRDPENPFELTNHAVLLVGYGTDAASGLDYWIVKNSWGTSWGENGYFRIRRGTDECAIESIALAATPIPKL

SEQ ID NO: 6 - protein, 437 aa, mouse
DTPANCTYPDLLGTWVFQVGPRSSRSDINCSVMEATEEKVVVHLKKLDTAYDELGNSGHFTLIYNQGFEIVLNDYKWFAFFKYEVRGHTAISYCHETMTGWVHDVLGRNWACFVGKKVESHIEKVNMNAAHLGGLQERYSERLYTHNHNFVKAINTVQSWTATAYKEYEKMSLRDLIRRSGHSQRIPRPKPAPMTDEIQQQILNLPESWDWRNVQGVNYVSPVRNQESCGSCYSFASMGMLEARIRILTNNSQTPILSPQEVVSCSPYAQGCDGGFPYLIAGKYAQDFGVVEESCFPYTAKDSPCKPRENCLRYYSSDYYYVGGFYGGCNEALMKLELVKGHPMAVAFEVHDDFLHYHSGIYHHTGLSDPFNPFELTNHAVLLVGYGRDPVTGIEYWIIKNSWGSNWGESGYFRIRRGTDECAIESIAVAAIPIPKL

SEQ ID NO: 7 - protein, 198 aa, chicken
  misc_feature (159)..(159): Xaa can be any naturally occurring amino acid
AQDSLGNFGFFTLIYNQGFEIVLNNYKWFAFFKYKKEGLNVTSYCNETLPGWVHDVLGHNWACFTGQKISSSSSDVHVRQLPLQKPRVGLSSRRFVHNFDFVNAINAHQKSWRATRYEEYENFSLEELTRRAGGLYSRTSRPKPAPLTPELLKKFRLTXSWDWRNVNGVNYVXRNNPVXRYHSCWHAEQILSKTPRAS

SEQ ID NO: 8 - protein, 194 aa, winter flounder
  misc_feature (175)..(175): Xaa can be any naturally occurring amino acid
NSARVINGYKWFAFFKYSEGGPTVTSYCDQTMPGWVHDVLGNNWACFVGKKVKPVPPRVDYKPLFSSRLLQKPYKNNMDFIDSINSVQSSWKAVAYPEHETFTLQELQRRAGGPASRVPMRVRPMPVRAGVAKMAAALPERFDWRNVGGVNFLSPVRNQASCGSCYSFAAMGDVXGSHPKSSPNNSXAPILQSR

SEQ ID NO: 9 - protein, 119 aa, zebrafish
  misc_feature (68)..(68): Xaa can be any naturally occurring amino acid
TPANCTYEDLLGTWIFSVSNVGQDKTINCSSTGQTVSTVTVDLQKLSVAVDDLGHTGFFTLIYNQSFXVVINDYKWFGFFKYTHHGSQEVSYCDQTLPGXVHDVLSNNXACNTGKKVQT

SEQ ID NO: 10 - protein, 434 aa, S. Japonicum
DTPANCSYMDAIGHWIFHVSRYKTKCTKQLDVSQTFSMNVQYPNIVTDSYGNMGKWTLIYNQFEITMNHRKWLIMFAYGPNNTYTCNKSMPMWTHDTLICQWHCFTATKVNHFQRMIEYKSPVLQLDGNQLYKVDTKFIKAINAKQNSWKATIYPEYSKYTIKEMRRRAGGSRSFKRQNVQLPKKNLTSAMMLELLALPKEFDWVNRPEGLRSPVTPVRNQKTCGSCYAFASTAAIEARIRLASRFRLQPILSPQDIIDCSPYSEGCDGGFPYLVAGKHGEDFGFVEEKCNPYTGVKSGTCNKLLGCTRYYTTDYHYIGGYYGATNEDLMKLELVKNGPFPVGFEVYGDFLQYKSGVYSHTDIINNHHPFNPFELTNHAVLLVGYGIDNSSNLPYWKIKNSWGQYWGEEGYFRILRGSDECGVQSIAIKFDVVL

SEQ ID NO: 11 - protein, 433 aa, S. mansoni
DTPANCTYEDAHGRWKFHIGDYQSKCPEKLNSKQSVVISLLYPDIAIDEFGNRGHWTLIYNQGFEVTINHRKWLVIFAYKSNGEFNCHKSMPMWTHDTLIDSGSVCSGKIGVHDKFHINKLFGSKSFGRTLYHINPSFVGINAHQKSWRGEIYPELSKYTIDELRNRAGGVKSMVTRPSVLNRKTPSKELISLTGNLPLEFDWTSPPDGSRSPVTPIRNQGICGSCYASPSAAALEARIRLVSNFSEQPILSPQTVVDCSPYSEGCNGGFPFLIAGKYGEDFGLPQKIVIPYTGEDTGKCTVSKNCTRYYTTDYSYIGGYYGATNEKLMQLELISNGPFPVGFEVYEDFQFYKEGIYHHTTVQTDHYNFNPFELTNHAVLLVGYGVDKLSGEPYWKVKNSWGVEWGEQGYFRILRGTDECGVESLGVRFDPVL

SEQ ID NO: 12 - protein, 119 aa, Artificial Sequence
  Residual propart domain
DTPANCTYLDLLGTWVFQVGSSGSQRDVNCSVMGPQEKKVVVYLQKLDTAYDDLGNSGHFTIIYNQGFEIVLNDYKWFAFFKYKEEGSKVTTYCNETMTGWVHDVLGRNWACFTGKKVK